SEQUENCE LISTING (81 sequences)

SEQ ID NO: 1 - DNA, 1698 bp, Artificial Sequence
  Synthetic Polynucleotide
atggccctgcctgtgacagctctgctcctccctctggccctgctgctccatgccgccagaccctgggcacacgtgggtggtcaggacaagcccttctgctctgcctggcccagcgctgtggtgcctcaaggaggacacgtgactcttcggtgtcactatcgtcgtgggttcaacatcttcacgctgtacaagaaagatggggtccctgtccctgagctctacaacagaatattctggaacagtttcctcattagccctgtgaccccagcacacgcagggacctacagatgtcgaggttttcacccgcactcccccactgagtggtcggcacccagcaaccccctggtgatcatggtcacaggtctatatgagaaaccttcgcttacagcccggctgggccccacggttcgcgcaggagagaacgtgaccttgtcctgcagctcccagagctcctttgacatctaccatctatccagggagggggaagcccatgaacttaggctccctgcagtgcccagcatcaatggaacattccaggccgacttccctctgggtcctgccacccacggagagacctacagatgcttcggctctttccatggatctccctacgagtggtcagacccgagtgacccactgcctgtttctgtcacaggaaacccttctagtagttggccttcacccactgaaccaagcttcaaaactggtatcgccagacacctgcatttttgggtgctggtggtggttggtggagtcctggcttgctatagcttgctagtaacagtggcctttattattttctgggtgacgcgtaagaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagc

SEQ ID NO: 2 - protein, 566 aa, Artificial Sequence
  Synthetic Polypeptide
MALPVTALLLPLALLLHAARPWAHVGGQDKPFCSAWPSAVVPQGGHVTLRCHYRRGFNIFTLYKKDGVPVPELYNRIFWNSFLISPVTPAHAGTYRCRGFHPHSPTEWSAPSNPLVIMVTGLYEKPSLTARLGPTVRAGENVTLSCSSQSSFDIYHLSREGEAHELRLPAVPSINGTFQADFPLGPATHGETYRCFGSFHGSPYEWSDPSDPLPVSVTGNPSSSWPSPTEPSFKTGIARHLHFWVLVVVGGVLACYSLLVTVAFIIFWVTRKKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - DNA, 1824 bp, Artificial Sequence
  Synthetic Polynucleotide
atggggatggcattgcctgttacagctctgctgctgcctctggctctgcttctgcatgctgctagaccttgggctcacgttggcggccaggataagcctttttgttctgcctggcctagcgccgtggttcctcaaggtggacacgtgaccctgcggtgtcactacagacggggcttcaacatcttcaccctgtacaagaaagacggcgtgcccgtgcctgagctgtacaacagaatcttctggaacagcttcctgatcagccccgtgacaccagctcacgccggcacatacagatgcagaggctttcaccctcacagccccacagagtggtccgctccatctaaccctctggtcatcatggtcaccggcctgtacgagaagcctagcctgacagctagactgggccctacagttagagccggcgagaatgtgaccctgtcctgtagcagccagagcagcttcgacatctaccacctgtctagagagggcgaagcccacgaactgagactgcctgccgtgcctagcatcaatggcaccttccaggccgattttccactgggacctgccacacacggcgagacttacagatgctttggcagcttccacggcagcccttacgagtggtctgatcctagcgatcctctgcctgtgtccgtgacaggcaatcctagcagcagctggccttctccaaccgagcctagctttaagaccggaatcgcccggcatctgcacaccacaacaccagcacctagacctccaactccagctcctacaatcgccagccagcctctgtctctgaggcctgaagcttgtagacctgctgctggcggagccgtgcataccagaggactggatttcgcctgcgatatctacatctgggcccctctggctggaacatgtggcgtgctgctgctgagcctcgtgatcacaaaacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 4 - protein, 607 aa, Artificial Sequence
  Synthetic Polypeptide
MGMALPVTALLLPLALLLHAARPWAHVGGQDKPFCSAWPSAVVPQGGHVTLRCHYRRGFNIFTLYKKDGVPVPELYNRIFWNSFLISPVTPAHAGTYRCRGFHPHSPTEWSAPSNPLVIMVTGLYEKPSLTARLGPTVRAGENVTLSCSSQSSFDIYHLSREGEAHELRLPAVPSINGTFQADFPLGPATHGETYRCFGSFHGSPYEWSDPSDPLPVSVTGNPSSSWPSPTEPSFKTGIARHLHTTTPAPRPPTPAPTIASQPLSLRPEACRPAAGGAVHTRGLDFACDIYIWAPLAGTCGVLLLSLVITKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 5 - DNA, 63 bp, Artificial Sequence
  Synthetic Polynucleotide
atggccctgcctgtgacagctctgctcctccctctggccctgctgctccatgccgccagaccc

SEQ ID NO: 6 - protein, 21 aa, Artificial Sequence
  Synthetic Polypeptide
MALPVTALLLPLALLLHAARP

SEQ ID NO: 7 - DNA, 663 bp, Artificial Sequence
  Synthetic Polynucleotide
tgggcacacgtgggtggtcaggacaagcccttctgctctgcctggcccagcgctgtggtgcctcaaggaggacacgtgactcttcggtgtcactatcgtcgtgggttcaacatcttcacgctgtacaagaaagatggggtccctgtccctgagctctacaacagaatattctggaacagtttcctcattagccctgtgaccccagcacacgcagggacctacagatgtcgaggttttcacccgcactcccccactgagtggtcggcacccagcaaccccctggtgatcatggtcacaggtctatatgagaaaccttcgcttacagcccggctgggccccacggttcgcgcaggagagaacgtgaccttgtcctgcagctcccagagctcctttgacatctaccatctatccagggagggggaagcccatgaacttaggctccctgcagtgcccagcatcaatggaacattccaggccgacttccctctgggtcctgccacccacggagagacctacagatgcttcggctctttccatggatctccctacgagtggtcagacccgagtgacccactgcctgtttctgtcacaggaaacccttctagtagttggccttcacccactgaaccaagcttcaaaactggtatcgccagacacctgcat

SEQ ID NO: 8 - DNA, 663 bp, Artificial Sequence
  Synthetic Polynucleotide
tgggctcacgttggcggccaggataagcctttttgttctgcctggcctagcgccgtggttcctcaaggtggacacgtgaccctgcggtgtcactacagacggggcttcaacatcttcaccctgtacaagaaagacggcgtgcccgtgcctgagctgtacaacagaatcttctggaacagcttcctgatcagccccgtgacaccagctcacgccggcacatacagatgcagaggctttcaccctcacagccccacagagtggtccgctccatctaaccctctggtcatcatggtcaccggcctgtacgagaagcctagcctgacagctagactgggccctacagttagagccggcgagaatgtgaccctgtcctgtagcagccagagcagcttcgacatctaccacctgtctagagagggcgaagcccacgaactgagactgcctgccgtgcctagcatcaatggcaccttccaggccgattttccactgggacctgccacacacggcgagacttacagatgctttggcagcttccacggcagcccttacgagtggtctgatcctagcgatcctctgcctgtgtccgtgacaggcaatcctagcagcagctggccttctccaaccgagcctagctttaagaccggaatcgcccggcatctgcac

SEQ ID NO: 9 - protein, 221 aa, Artificial Sequence
  Synthetic Polypeptide
WAHVGGQDKPFCSAWPSAVVPQGGHVTLRCHYRRGFNIFTLYKKDGVPVPELYNRIFWNSFLISPVTPAHAGTYRCRGFHPHSPTEWSAPSNPLVIMVTGLYEKPSLTARLGPTVRAGENVTLSCSSQSSFDIYHLSREGEAHELRLPAVPSINGTFQADFPLGPATHGETYRCFGSFHGSPYEWSDPSDPLPVSVTGNPSSSWPSPTEPSFKTGIARHLH

SEQ ID NO: 10 - protein, 221 aa, Artificial Sequence
  Synthetic Polypeptide
WAHVGGQDKPFCSAWPSAVVPQGGHVTLRCHYRRGFNIFTLYKKDGVPVPELYNRIFWNSFLISPVTPAHAGTYRCRGFHPHSPTEWSAPSNPLVIMVTGLYEKPSLTARPGPTVRAGENVTLSCSSQSSFDIYHLSREGEAHELRLPAVPSINGTFQADFPLGPATHGETYRCFGSFHGSPYEWSDPSDPLPVSVTGNPSSSWPSPTEPSFKTGIARHLH

SEQ ID NO: 11 - DNA, 81 bp, Artificial Sequence
  Synthetic Polynucleotide
ttttgggtgctggtggtggttggtggagtcctggcttgctatagcttgctagtaacagtggcctttattattttctgggtg

SEQ ID NO: 12 - protein, 27 aa, Artificial Sequence
  Synthetic Polypeptide
FWVLVVVGGVLACYSLLVTVAFIIFWV

SEQ ID NO: 13 - DNA, 198 bp, Artificial Sequence
  Synthetic Polynucleotide
accacaacaccagcacctagacctccaactccagctcctacaatcgccagccagcctctgtctctgaggcctgaagcttgtagacctgctgctggcggagccgtgcataccagaggactggatttcgcctgcgatatctacatctgggcccctctggctggaacatgtggcgtgctgctgctgagcctcgtgatcaca

SEQ ID NO: 14 - protein, 66 aa, Artificial Sequence
  Synthetic Polypeptide
TTTPAPRPPTPAPTIASQPLSLRPEACRPAAGGAVHTRGLDFACDIYIWAPLAGTCGVLLLSLVIT

SEQ ID NO: 15 - DNA, 342 bp, Artificial Sequence
  Synthetic Polynucleotide
acgcgtaagaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgga

SEQ ID NO: 16 - protein, 114 aa, Artificial Sequence
  Synthetic Polypeptide
TRKKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRG

SEQ ID NO: 17 - DNA, 342 bp, Artificial Sequence
  Synthetic Polynucleotide
aaacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgga

SEQ ID NO: 18 - protein, 114 aa, Artificial Sequence
  Synthetic Polypeptide
KRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRG

SEQ ID NO: 19 - DNA, 60 bp, Artificial Sequence
  Synthetic Polynucleotide
cagtgtactaattatgctctcttgaaattggctggagatgttgagagcaatcccgggccc

SEQ ID NO: 20 - protein, 20 aa, Artificial Sequence
  Synthetic Polypeptide
QCTNYALLKLAGDVESNPGP

SEQ ID NO: 21 - DNA, 490 bp, Artificial Sequence
  Synthetic Polynucleotide
gcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctgacaatt

SEQ ID NO: 22 - protein, 160 aa, Artificial Sequence
  Synthetic Polypeptide
ISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 23 - DNA, 123 bp, Artificial Sequence
  Synthetic Polynucleotide
agaagtaagagaagtagactactacacagtgactacatgaacatgacgccgagaagaccggggccgacgagaaagcactaccagccgtacgcgccgccgagagacttcgcggcgtacagaagt

SEQ ID NO: 24 - protein, 41 aa, Artificial Sequence
  Synthetic Polypeptide
RSKRSRLLHSDYMNMTPRRPGPTRKHYQPYAPPRDFAAYRS

SEQ ID NO: 25 - DNA, 81 bp, Artificial Sequence
  Synthetic Polynucleotide
atcatctccttctttcttgcgctgacgtcgactgcgttgctcttcctgctgttcttcctcacgctccgtttctctgttgtt

SEQ ID NO: 26 - protein, 27 aa, Artificial Sequence
  Synthetic Polypeptide
IISFFLALTSTALLFLLFFLTLRFSVV

SEQ ID NO: 27 - DNA, 63 bp, Artificial Sequence
  Synthetic Polynucleotide
ctcctggcaggcctcgtggctgctgatgcggtggcatcgctgctcatcgtgggggcggtgttc

SEQ ID NO: 28 - protein, 21 aa, Artificial Sequence
  Synthetic Polypeptide
LLAGLVAADAVASLLIVGAVF

SEQ ID NO: 29 - DNA, 63 bp, Artificial Sequence
  Synthetic Polynucleotide
ggcgtgctggcagggatcgtgatgggagacctggtgctgacagtgctcattgccctggccgtg

SEQ ID NO: 30 - protein, 21 aa, Artificial Sequence
  Synthetic Polypeptide
GVLAGIVMGDLVLTVLIALAV

SEQ ID NO: 31 - DNA, 63 bp, Artificial Sequence
  Synthetic Polynucleotide
gcggtgatgattatttttcgcattggcatggcggtggcgattttttgctgctttttttttccg

SEQ ID NO: 32 - protein, 21 aa, Artificial Sequence
  Synthetic Polypeptide
AVMIIFRIGMAVAIFCCFFFP

SEQ ID NO: 33 - DNA, 126 bp, Artificial Sequence
  Synthetic Polynucleotide
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 34 - protein, 42 aa, Artificial Sequence
  Synthetic Polypeptide
KRGRKKLLYIFKQPFMRPVQTTQEEDGCSCRFPEEEEGGCEL

SEQ ID NO: 35 - DNA, 72 bp, Artificial Sequence
  Synthetic Polynucleotide
ctttgcgcacgcccacgccgcagccccgcccaagaagatggcaaagtctacatcaacatgccaggcaggggc

SEQ ID NO: 36 - protein, 24 aa, Artificial Sequence
  Synthetic Polypeptide
LCARPRRSPAQEDGKVYINMPGRG

SEQ ID NO: 37 - DNA, 156 bp, Artificial Sequence
  Synthetic Polynucleotide
tacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaa

SEQ ID NO: 38 - protein, 52 aa, Artificial Sequence
  Synthetic Polypeptide
YFLGRLVPRGRGAAEAATRKQRITETESPYQELQGQRSDVYSDLNTQRPYYK

SEQ ID NO: 39 - DNA, 153 bp, Artificial Sequence
  Synthetic Polynucleotide
agcgcgaacgaacgctgcaaaagcaaagtggtgccgtgccgccagaaacagtggcgcaccagctttgatagcaaaaaactggatctgaactataaccattttgaaagcatggaatggagccatcgcagccgccgcggccgcatttggggcatg

SEQ ID NO: 40 - protein, 51 aa, Artificial Sequence
  Synthetic Polypeptide
SANERCKSKVVPCRQKQWRTSFDSKKLDLNYNHFESMEWSHRSRRGRIWGM

SEQ ID NO: 41 - DNA, 742 bp, Artificial Sequence
  Synthetic Polynucleotide
gtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaatt

SEQ ID NO: 42 - protein, 227 aa, Artificial Sequence
  Synthetic Polypeptide
TVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEAL

SEQ ID NO: 43 - DNA, 117 bp, Artificial Sequence
  Synthetic Polynucleotide
attgaagttatgtatcctcctccttacctagacaatgagaagagcaatggaaccattatccatgtgaaagggaaacacctttgtccaagtcccctatttcccggaccttctaagccc

SEQ ID NO: 44 - protein, 39 aa, Artificial Sequence
  Synthetic Polypeptide
IEVMYPPPYLDNEKSNGTIIHVKGKHLCPSPLFPGPSKP

SEQ ID NO: 45 - protein, 18 aa, Artificial Sequence
  Synthetic Polypeptide
EGRGSLLTCGDVEENPGP

SEQ ID NO: 46 - protein, 19 aa, Artificial Sequence
  Synthetic Polypeptide
ATNFSLLKQAGDVEENPGP

SEQ ID NO: 47 - protein, 22 aa, Artificial Sequence
  Synthetic Polypeptide
VKQTLNFDLLKLAGDVESNPGP

SEQ ID NO: 48 - protein, 438 aa, Artificial Sequence
  Synthetic Polypeptide
GHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGV

SEQ ID NO: 49 - protein, 390 aa, Artificial Sequence
  Synthetic Polypeptide
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGV

SEQ ID NO: 50 - protein, 217 aa, Artificial Sequence
  Description of Artificial Sequence Synthetic polypeptide
DIVLTQSPASLDVSLGQRATISCRASKSVSTSGYSYMHWYQQKPGQSPKLLIYLASNRESGVPARFSGSGSGTDFTLNIHPVEEEDAATYYCQHSREFPTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 51 - protein, 15 aa, Artificial Sequence
  Description of Artificial Sequence Synthetic peptide
RASKSVSTSGYSYMH

SEQ ID NO: 52 - protein, 7 aa, Artificial Sequence
  Description of Artificial Sequence Synthetic peptide
LASNRES

SEQ ID NO: 53 - protein, 8 aa, Artificial Sequence
  Description of Artificial Sequence Synthetic peptide
QHSREFPT

SEQ ID NO: 54 - protein, 110 aa, Artificial Sequence
  Description of Artificial Sequence Synthetic polypeptide
DIVLTQSPASLDVSLGQRATISCRASKSVSTSGYSYMHWYQQKPGQSPKLLIYLASNRESGVPARFSGSGSGTDFTLNIHPVEEEDAATYYCQHSREFPTFGAGTKLELK

SEQ ID NO: 55 - DNA, 330 bp, Artificial Sequence
  Description of Artificial Sequence Synthetic polynucleotide
gatattgtgctgacccagagcccggcgagcctggatgtgagcctgggccagcgcgcgaccattagctgccgcgcgagcaaaagcgtgagcaccagcggctatagctatatgcattggtatcagcagaaaccgggccagagcccgaaactgctgatttatctggcgagcaaccgcgaaagcggcgtgccggcgcgctttagcggcagcggcagcggcaccgattttaccctgaacattcatccggtggaagaagaagatgcggcgacctattattgccagcatagccgcgaatttccgacctttggcgcgggcaccaaactggaactgaaa

SEQ ID NO: 56 - protein, 444 aa, Artificial Sequence
  Description of Artificial Sequence Synthetic polypeptide
KVQLVESGGGLVKPGGSLKLSCSASGFPFSDYYMYWVRQTPEKRLEWVATISDDDDYTYYPDSMKGRFTISRDNAKNNLYLQMSSLKSEDTAMYYCSRGIYYGSSPFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 57 - protein, 7 aa, Artificial Sequence
  Description of Artificial Sequence Synthetic peptide
GFPFSDY

SEQ ID NO: 58 - protein, 6 aa, Artificial Sequence
  Description of Artificial Sequence Synthetic peptide
SDDDDY

SEQ ID NO: 59 - protein, 11 aa, Artificial Sequence
  Description of Artificial Sequence Synthetic peptide
GIYYGSSPFAY

SEQ ID NO: 60 - protein, 120 aa, Artificial Sequence
  Description of Artificial Sequence Synthetic polypeptide
KVQLVESGGGLVKPGGSLKLSCSASGFPFSDYYMYWVRQTPEKRLEWVATISDDDDYTYYPDSMKGRFTISRDNAKNNLYLQMSSLKSEDTAMYYCSRGIYYGSSPFAYWGQGTLVTVSA

SEQ ID NO: 61 - DNA, 360 bp, Artificial Sequence
  Description of Artificial Sequence Synthetic polynucleotide
aaagtgcagctggtggaaagcggcggcggcctggtgaaaccgggcggcagcctgaaactgagctgcagcgcgagcggctttccgtttagcgattattatatgtattgggtgcgccagaccccggaaaaacgcctggaatgggtggcgaccattagcgatgatgatgattatacctattatccggatagcatgaaaggccgctttaccattagccgcgataacgcgaaaaacaacctgtatctgcagatgagcagcctgaaaagcgaagataccgcgatgtattattgcagccgcggcatttattatggcagcagcccgtttgcgtattggggccagggcaccctggtgaccgtgagcgcg

SEQ ID NO: 62 - protein, 214 aa, Artificial Sequence
  Description of Artificial Sequence Synthetic polypeptide
ETTVTQSPASLSVATGEKVTIRCITSTDIDDDMNWYQQKPGEPPKLLISEDNILRPGVPSRFSSSGYGTDFVFTIENTLSEDVADYYCLQSDNMPLTFGGGTRLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 63 - protein, 11 aa, Artificial Sequence
  Description of Artificial Sequence Synthetic peptide
ITSTDIDDDMN

SEQ ID NO: 64 - protein, 7 aa, Artificial Sequence
  Description of Artificial Sequence Synthetic peptide
EDNILRP

SEQ ID NO: 65 - protein, 9 aa, Artificial Sequence
  Description of Artificial Sequence Synthetic peptide
LQSDNMPLT

SEQ ID NO: 66 - protein, 107 aa, Artificial Sequence
  Description of Artificial Sequence Synthetic polypeptide
ETTVTQSPASLSVATGEKVTIRCITSTDIDDDMNWYQQKPGEPPKLLISEDNILRPGVPSRFSSSGYGTDFVFTIENTLSEDVADYYCLQSDNMPLTFGGGTRLEIK

SEQ ID NO: 67 - DNA, 321 bp, Artificial Sequence
  Description of Artificial Sequence Synthetic polynucleotide
gaaaccaccgtgacccagagcccggcgagcctgagcgtggcgaccggcgaaaaagtgaccattcgctgcattaccagcaccgatattgatgatgatatgaactggtatcagcagaaaccgggcgaaccgccgaaactgctgattagcgaagataacattctgcgcccgggcgtgccgagccgctttagcagcagcggctatggcaccgattttgtgtttaccattgaaaacaccctgagcgaagatgtggcggattattattgcctgcagagcgataacatgccgctgacctttggcggcggcacccgcctggaaattaaa

SEQ ID NO: 68 - protein, 454 aa, Artificial Sequence
  Description of Artificial Sequence Synthetic polypeptide
EVKLVESGGSLVQPGGSLKLSCAASGFSFSSYTMSWVRQTPKKRLEWVAYVSNGAGTTYYPDSLKGRFTISRDNAKNTLHLLMTSLKSEDTAIYYCARHYYGSYHFDYWGQGTTLIVSSAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 69 - protein, 7 aa, Artificial Sequence
  Description of Artificial Sequence Synthetic peptide
GFSFSSY

SEQ ID NO: 70 - protein, 6 aa, Artificial Sequence
  Description of Artificial Sequence Synthetic peptide
SNGAGT

SEQ ID NO: 71 - protein, 10 aa, Artificial Sequence
  Description of Artificial Sequence Synthetic peptide
HYYGSYHFDY

SEQ ID NO: 72 - protein, 119 aa, Artificial Sequence
  Description of Artificial Sequence Synthetic polypeptide
EVKLVESGGSLVQPGGSLKLSCAASGFSFSSYTMSWVRQTPKKRLEWVAYVSNGAGTTYYPDSLKGRFTISRDNAKNTLHLLMTSLKSEDTAIYYCARHYYGSYHFDYWGQGTTLIVSS

SEQ ID NO: 73 - DNA, 357 bp, Artificial Sequence
  Description of Artificial Sequence Synthetic polynucleotide
gaagtgaaactggtggaaagcggcggcagcctggtgcagccgggcggcagcctgaaactgagctgcgcggcgagcggctttagctttagcagctataccatgagctgggtgcgccagaccccgaaaaaacgcctggaatgggtggcgtatgtgagcaacggcgcgggcaccacctattatccggatagcctgaaaggccgctttaccattagccgcgataacgcgaaaaacaccctgcatctgctgatgaccagcctgaaaagcgaagataccgcgatttattattgcgcgcgccattattatggcagctatcattttgattattggggccagggcaccaccctgattgtgagcagc

SEQ ID NO: 74 - DNA, 3309 bp, Artificial Sequence
  Description of Artificial Sequence Synthetic polynucleotide
atgctcgagggagtgcaggtggaaaccatctccccaggcgacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagatcatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagcttcgcgagccgagggcaggggaagtcttctaacatgcggggacgtggaggaaaatcccgggcccatggagtttgggctgagctggctttttcttgtggctattttaaaaggtgtccagtgctctagagagaaagtgcagctggtggaaagcggcggcggcctggtgaaaccgggcggcagcctgaaactgagctgcagcgcgagcggctttccgtttagcgattattatatgtattgggtgcgccagaccccggaaaaacgcctggaatgggtggcgaccattagcgatgatgatgattatacctattatccggatagcatgaaaggccgctttaccattagccgcgataacgcgaaaaacaacctgtatctgcagatgagcagcctgaaaagcgaagataccgcgatgtattattgcagccgcggcatttattatggcagcagcccgtttgcgtattggggccagggcaccctggtgaccgtgagcgcgttggaaataaagggctctacaagcggctcaggaaaacctggatcaggcgaagggtctacggatattgtgctgacccagagcccggcgagcctggatgtgagcctgggccagcgcgcgaccattagctgccgcgcgagcaaaagcgtgagcaccagcggctatagctatatgcattggtatcagcagaaaccgggccagagcccgaaactgctgatttatctggcgagcaaccgcgaaagcggcgtgccggcgcgctttagcggcagcggcagcggcaccgattttaccctgaacattcatccggtggaagaagaagatgcggcgacctattattgccagcatagccgcgaatttccgacctttggcgcgggcaccaaactggaactgaaacacgtacgccccattgaagttatgtatcctcctccttacctagacaatgagaagagcaatggaaccattatccatgtgaaagggaaacacctttgtccaagtcccctatttcccggaccttctaagcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 75 - protein, 1102 aa, Artificial Sequence
  Description of Artificial Sequence Synthetic polypeptide
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRAEGRGSLLTCGDVEENPGPMEFGLSWLFLVAILKGVQCSREKVQLVESGGGLVKPGGSLKLSCSASGFPFSDYYMYWVRQTPEKRLEWVATISDDDDYTYYPDSMKGRFTISRDNAKNNLYLQMSSLKSEDTAMYYCSRGIYYGSSPFAYWGQGTLVTVSALEIKGSTSGSGKPGSGEGSTDIVLTQSPASLDVSLGQRATISCRASKSVSTSGYSYMHWYQQKPGQSPKLLIYLASNRESGVPARFSGSGSGTDFTLNIHPVEEEDAATYYCQHSREFPTFGAGTKLELKHVRPIEVMYPPPYLDNEKSNGTIIHVKGKHLCPSPLFPGPSKPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 76 - DNA, 3255 bp, Artificial Sequence
  Description of Artificial Sequence Synthetic polynucleotide
atgctcgagggagtgcaggtggaaaccatctccccaggcgacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagatcatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagcttcgcgagccgagggcaggggaagtcttctaacatgcggggacgtggaggaaaatcccgggcccatggagtttgggctgagctggctttttcttgtggctattttaaaaggtgtccagtgctctagagagaaagtgcagctggtggaaagcggcggcggcctggtgaaaccgggcggcagcctgaaactgagctgcagcgcgagcggctttccgtttagcgattattatatgtattgggtgcgccagaccccggaaaaacgcctggaatgggtggcgaccattagcgatgatgatgattatacctattatccggatagcatgaaaggccgctttaccattagccgcgataacgcgaaaaacaacctgtatctgcagatgagcagcctgaaaagcgaagataccgcgatgtattattgcagccgcggcatttattatggcagcagcccgtttgcgtattggggccagggcaccctggtgaccgtgagcgcgttggaaataaagggctctacaagcggctcaggaaaacctggatcaggcgaagggtctacggatattgtgctgacccagagcccggcgagcctggatgtgagcctgggccagcgcgcgaccattagctgccgcgcgagcaaaagcgtgagcaccagcggctatagctatatgcattggtatcagcagaaaccgggccagagcccgaaactgctgatttatctggcgagcaaccgcgaaagcggcgtgccggcgcgctttagcggcagcggcagcggcaccgattttaccctgaacattcatccggtggaagaagaagatgcggcgacctattattgccagcatagccgcgaatttccgacctttggcgcgggcaccaaactggaactgaaaccgtacgccattgaagttatgtatcctcctccttacctagacaatgagaagagcaatggaaccattatccatgtgaaagggaaacacctttgtccaagtcccctatttcccggaccttctaagcccaaattttgggtgctggtggtggttggtggagtcctggcttgctatagcttgctagtaacagtggcctttattattttctgggtgctttgcgcacgcccacgccgcagccccgcccaagaagatggcaaagtctacatcaacatgccaggcaggggccgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 77 - protein, 1084 aa, Artificial Sequence
  Description of Artificial Sequence Synthetic polypeptide
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRAEGRGSLLTCGDVEENPGPMEFGLSWLFLVAILKGVQCSREKVQLVESGGGLVKPGGSLKLSCSASGFPFSDYYMYWVRQTPEKRLEWVATISDDDDYTYYPDSMKGRFTISRDNAKNNLYLQMSSLKSEDTAMYYCSRGIYYGSSPFAYWGQGTLVTVSALEIKGSTSGSGKPGSGEGSTDIVLTQSPASLDVSLGQRATISCRASKSVSTSGYSYMHWYQQKPGQSPKLLIYLASNRESGVPARFSGSGSGTDFTLNIHPVEEEDAATYYCQHSREFPTFGAGTKLELKPYAIEVMYPPPYLDNEKSNGTIIHVKGKHLCPSPLFPGPSKPKFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 78 - DNA, 3297 bp, Artificial Sequence
  Description of Artificial Sequence Synthetic polynucleotide
atgctcgagggagtgcaggtggaaaccatctccccaggcgacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagatcatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagcttcgcgagccgagggcaggggaagtcttctaacatgcggggacgtggaggaaaatcccgggcccatggagtttgggctgagctggctttttcttgtggctattttaaaaggtgtccagtgctctagagaggaagtgaaactggtggaaagcggcggcagcctggtgcagccgggcggcagcctgaaactgagctgcgcggcgagcggctttagctttagcagctataccatgagctgggtgcgccagaccccgaaaaaacgcctggaatgggtggcgtatgtgagcaacggcgcgggcaccacctattatccggatagcctgaaaggccgctttaccattagccgcgataacgcgaaaaacaccctgcatctgctgatgaccagcctgaaaagcgaagataccgcgatttattattgcgcgcgccattattatggcagctatcattttgattattggggccagggcaccaccctgattgtgagcagcttggaaataaagggctctacaagcggctcaggaaaacctggatcaggcgaagggtctacggaaaccaccgtgacccagagcccggcgagcctgagcgtggcgaccggcgaaaaagtgaccattcgctgcattaccagcaccgatattgatgatgatatgaactggtatcagcagaaaccgggcgaaccgccgaaactgctgattagcgaagataacattctgcgcccgggcgtgccgagccgctttagcagcagcggctatggcaccgattttgtgtttaccattgaaaacaccctgagcgaagatgtggcggattattattgcctgcagagcgataacatgccgctgacctttggcggcggcacccgcctggaaattaaacacgtacgccccattgaagttatgtatcctcctccttacctagacaatgagaagagcaatggaaccattatccatgtgaaagggaaacacctttgtccaagtcccctatttcccggaccttctaagcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 79 - protein, 1098 aa, Artificial Sequence
  Description of Artificial Sequence Synthetic polypeptide
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRAEGRGSLLTCGDVEENPGPMEFGLSWLFLVAILKGVQCSREEVKLVESGGSLVQPGGSLKLSCAASGFSFSSYTMSWVRQTPKKRLEWVAYVSNGAGTTYYPDSLKGRFTISRDNAKNTLHLLMTSLKSEDTAIYYCARHYYGSYHFDYWGQGTTLIVSSLEIKGSTSGSGKPGSGEGSTETTVTQSPASLSVATGEKVTIRCITSTDIDDDMNWYQQKPGEPPKLLISEDNILRPGVPSRFSSSGYGTDFVFTIENTLSEDVADYYCLQSDNMPLTFGGGTRLEIKHVRPIEVMYPPPYLDNEKSNGTIIHVKGKHLCPSPLFPGPSKPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 80 - DNA, 3243 bp, Artificial Sequence
  Description of Artificial Sequence Synthetic polynucleotide
atgctcgagggagtgcaggtggaaaccatctccccaggcgacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagatcatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagcttcgcgagccgagggcaggggaagtcttctaacatgcggggacgtggaggaaaatcccgggcccatggagtttgggctgagctggctttttcttgtggctattttaaaaggtgtccagtgctctagagaggaagtgaaactggtggaaagcggcggcagcctggtgcagccgggcggcagcctgaaactgagctgcgcggcgagcggctttagctttagcagctataccatgagctgggtgcgccagaccccgaaaaaacgcctggaatgggtggcgtatgtgagcaacggcgcgggcaccacctattatccggatagcctgaaaggccgctttaccattagccgcgataacgcgaaaaacaccctgcatctgctgatgaccagcctgaaaagcgaagataccgcgatttattattgcgcgcgccattattatggcagctatcattttgattattggggccagggcaccaccctgattgtgagcagcttggaaataaagggctctacaagcggctcaggaaaacctggatcaggcgaagggtctacggaaaccaccgtgacccagagcccggcgagcctgagcgtggcgaccggcgaaaaagtgaccattcgctgcattaccagcaccgatattgatgatgatatgaactggtatcagcagaaaccgggcgaaccgccgaaactgctgattagcgaagataacattctgcgcccgggcgtgccgagccgctttagcagcagcggctatggcaccgattttgtgtttaccattgaaaacaccctgagcgaagatgtggcggattattattgcctgcagagcgataacatgccgctgacctttggcggcggcacccgcctggaaattaaaccgtacgccattgaagttatgtatcctcctccttacctagacaatgagaagagcaatggaaccattatccatgtgaaagggaaacacctttgtccaagtcccctatttcccggaccttctaagcccaaattttgggtgctggtggtggttggtggagtcctggcttgctatagcttgctagtaacagtggcctttattattttctgggtgctttgcgcacgcccacgccgcagccccgcccaagaagatggcaaagtctacatcaacatgccaggcaggggccgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 81 - protein, 1080 aa, Artificial Sequence
  Description of Artificial Sequence Synthetic polypeptide
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRAEGRGSLLTCGDVEENPGPMEFGLSWLFLVAILKGVQCSREEVKLVESGGSLVQPGGSLKLSCAASGFSFSSYTMSWVRQTPKKRLEWVAYVSNGAGTTYYPDSLKGRFTISRDNAKNTLHLLMTSLKSEDTAIYYCARHYYGSYHFDYWGQGTTLIVSSLEIKGSTSGSGKPGSGEGSTETTVTQSPASLSVATGEKVTIRCITSTDIDDDMNWYQQKPGEPPKLLISEDNILRPGVPSRFSSSGYGTDFVFTIENTLSEDVADYYCLQSDNMPLTFGGGTRLEIKPYAIEVMYPPPYLDNEKSNGTIIHVKGKHLCPSPLFPGPSKPKFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

(19) United States
(12) Patent Application Publication
(10) Pub. No.: US 2024/0252539 A1
(43) Pub. Date: Aug. 1, 2024

(54) CHIMERIC ANTIGEN RECEPTOR TO TARGET HLA-G-POSITIVE CANCERS

(71) Applicant: BOARD OF REGENTS, THE UNIVERSITY OF TEXAS SYSTEM, Austin, TX (US)

(72) Inventors: Katy REZVANI, Houston, TX (US); Elizabeth J. SHPALL, Houston, TX (US); Rafet BASAR, Houston, TX (US); Sunil ACHARYA, Houston, TX (US); Nadima UPRETY, Houston, TX (US); David MARIN COSTA, Houston, TX (US); Emily ENSLEY, Houston, TX (US)

(73) Assignee: BOARD OF REGENTS, THE UNIVERSITY OF TEXAS SYSTEM, Austin, TX (US)

(21) Appl. No.: 18/560,791

(22) PCT Filed: May 26, 2022
(86) PCT No.: PCT/US2022/031144

§ 371 Date: Nov. 14, 2023

Related U.S. Application Data
(60) Provisional application No. 63/193,515, filed on May 26, 2021.

(51) Int. Cl. A61K 35/17; A61K 39/00; A61K 45/06; A61K 47/68; A61P 35/00; C07K 14/54; C07K 16/28; C12N 15/85

(52) U.S. Cl. CPC A61K 35/17; A61K 39/4613; A61K 39/4631; A61K 39/464411; A61K 39/46444; A61K 45/06; A61K 47/6829; A61P 35/00; C07K 14/5443; C07K 16/2833; C12N 15/85; A61K 2239/13; A61K 2239/21; A61K 2239/22

(57) ABSTRACT

Embodiments of the disclosure include methods and compositions related to targeting of HLA-G-expressing cells with particular engineered receptors. In specific embodiments, NK cells are specifically engineered to bind HLA-G using particular chimeric antigen receptor constructs. In certain embodiments, vectors that express the HLA-G-targeting CARs also express particular a suicide gene and/or one or more particular cytokines.

This application claims priority to U.S. Provisional Patent Application Ser. No. 63/193,515, filed May 26, 2021, which is incorporated by reference herein in its entirety.

### BACKGROUND

### I. Technical Field

Embodiments of the disclosure include at least the fields of cell biology, molecular biology, immunology, and medicine, including cancer medicine.

### II. Background

Genetic reprogramming of Natural Killer (NK) cells for adoptive cancer immunotherapy has clinically relevant applications and benefits such as 1) innate anti-tumor surveillance without prior need for sensitization; 2) allogeneic efficacy without graft versus host reactivity; and 3) direct cell-mediated cytotoxicity and cytolysis of target tumors. Human NK cell development and acquisition of self-tolerance, alloreactivity, and effector functions is an adaptive process of licensing, calibration, and arming. At the molecular level, specific activating and inhibitory receptors direct NK cellular functions by aggregating, balancing, and integrating extracellular signals into distinct effector functions. The functional activity of NK cells and responsiveness to extrinsic stimuli follow the ‘rheostat’ model of continuous education and thus are amenable to reprogramming. Genetic modification of NK cells to redirect their effector functions is an effective method to harness their cytotoxic capability to kill tumor cells.

HLA-G is a non-classical HLA class I molecule and in the healthy adult, is only expressed in the placenta. HLA-G interacts with receptors on NK cells, T cells, B cells, monocytes and dendritic cells, inhibiting their function and creating a state of immune tolerance between the mother and the fetus. HLA-G is overexpressed by many hematologic and solid tumors. Cancer cells aberrantly express HLA-G to escape immune surveillance by engaging with the inhibitory receptors KIR2DL4, leukocyte immunoglobulin-like receptor subfamily B member 1 (LILRB1), and leukocyte immunoglobulin-like receptor subfamily B member 2 (LILRB2) on NK cells.

The present disclosure, in particular embodiments, concerns methods and compositions concerning genetic engineering of cells, including human NK cells, for cell therapy to target cancers including HLA-G-positive tumors.

### BRIEF SUMMARY

Embodiments of the disclosure encompass methods and compositions related to engineered cellular receptors, including chimeric antigen receptors (CARs) that target HLA-G (also known as HLA class I histocompatibility antigen, alpha chain G, HLA G antigen, or MHC class I antigen G, for example). In specific embodiments, the engineered receptors that target HLA-G are in the form of polynucleotides, polypeptides, and/or are comprised on the surface of cells of any kind, including immune cells. In specific cases, the cells are immune cells, and in certain embodiments the immune cells are NK cells, NK T cells, invariant NKT cells, gamma delta T cells, alpha beta T cells, regulatory T cells, B cells, macrophages, mesenchymal stromal cells (MSCs), dendritic cells, and so forth, from any source. In some embodiments, the immune cells are NK cells. In certain embodiments, reprogrammed NK cells from cord blood (CB-NK) are encompassed for targeting cancers expressing HLA-G molecules.

HLA-G is utilized as a target antigen for aspects of the disclosed methods and compositions at least in part because it is expressed on multiple cancers, including breast, colorectal, cervical, esophageal, Ewing, gastric, glioblastoma, hepatocellular, lung, nasopharyngeal ovarian and thyroid cancer. In addition, HLA-G overexpression is involved in cancer immune evasion, and HLA-G can be considered an immune checkpoint. Thus, its targeting can be used to overcome this important mechanism of tumor immune escape.

The present disclosure includes a number of novel CAR molecules including, in specific cases, fusion of an scFv targeting human HLA-G (including, for example, an scFv from an antibody clone such as G233, 26-2H11, MEM-G/1, MEM-G/9, MEM-G/11, MEM-G/13, 1B8, 5E6H7, 1-2C3, 16G1, 5A6G7, 87G, or 3C/G4) or of an extracellular domain from KIR2DL4, LILRB1, or LILRB2 with one or more activating signaling endodomains, including in some cases that incorporate either CD3 alone or in combination with costimulatory or adaptor signaling domains, such as from NKG2D, OX-40, CD27, 41BB, CD28, DAP10, DAP12, and/or 2B4. In specific cases, allogeneic CB-NK cells are retrovirally transduced to express a HLA-G CAR. In particular embodiments, immune cells of the disclosure encompassing HLA-G CAR molecules also express one or more proteins that support their survival and proliferation. In specific cases, the immune cells are engineered to express one or more cytokines that facilitate the cells' expansion and persistence. In specific cases, the one or more cytokines are interleukin 15 (IL-15), IL-2, IL-7, IL-12, IL-18, IL-21, and/or IL-23. In certain aspects, a vector that encodes the CAR also encodes the cytokine, and each ultimately are produced as separate polypeptides. In other aspects, the CAR and the cytokine are encoded on separate vectors.

Particular embodiments of the disclosure allow for the use of off-the-shelf immune cells, including at least NK cells, that are allogeneic with respect to a recipient individual, that target HLA-G-positive cells of any kind, and that also may or may not be transduced to express one or more cytokines, such as IL-15, IL-2, IL-21, IL-12, IL-23, IL-7, and/or IL-18.

In specific embodiments of the disclosure, expression of one or more endogenous genes in the immune cell has been modified, for example the expression may be partially or fully reduced in expression. Although the modification may occur by any means, in specific embodiments expression of the one or more genes has been modified, such as by being reduced in expression levels, and this may occur by any suitable means including at least CRISPR. Merely as examples, the endogenous gene may be selected from the group consisting of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, CD5, CD7, CTLA-4, TDAG8, CD38, and a combination thereof.

Embodiments of the disclosure include polynucleotides that encode an anti-HLA-G chimeric antigen receptor (CAR) and that also may encode one or more cytokines. In specific embodiments, the polynucleotide encodes a CAR comprising a KIR2DL4 extracellular domain, a transmembrane domain, and an intracellular domain that is not a KIR2DL4 intracellular domain. In some embodiments, the CAR comprises all of an extracellular domain of a KIR2DL4 protein. Alternatively, the CAR comprises a HLA-G-binding portion of an extracellular domain of a KIR2DL4 protein. In specific embodiments, the KIR2DL4 extracellular domain comprises SEQ ID NO:9. In certain aspects, the KIR2DL4 extracellular domain is encoded by SEQ ID NO:7. In some embodiments, the KIR2DL4 extracellular domain comprises SEQ ID NO:10. In some cases, the KIR2DL4 extracellular domain is a codon optimized KIR2DL4 extracellular domain. In certain aspects, the KIR2DL4 extracellular domain is encoded by SEQ ID NO:8. In some embodiments, the CAR comprises a portion of a KIR2DL4 intracellular domain, for example a portion of a KIR2DL4 intracellular domain capable of mediating an activating signal.

Additional embodiments of the disclosure include polynucleotides that encode an anti-HLA-G CAR and that also may encode one or more cytokines. In specific embodiments, the polynucleotide encodes a CAR comprising a LILRB1 extracellular domain, a transmembrane domain, and an intracellular domain that is not a LILRB1 intracellular domain. In some embodiments, the CAR comprises all of an extracellular domain of a LILRB1 protein. Alternatively, the CAR comprises a HLA-G-binding portion of an extracellular domain of a LILRB1 protein. In some embodiments, the CAR comprises a portion of a KIR2DL4 intracellular domain, for example a portion of a LILRB1 intracellular domain capable of mediating an activating signal. Further embodiments of the disclosure include polynucleotides that encode an anti-HLA-G CAR, the CAR comprising a LILRB2 extracellular domain, a transmembrane domain, and an intracellular domain that is not a LILRB2 intracellular domain. In some embodiments, the CAR comprises all of an extracellular domain of a LILRB2 protein. Alternatively, the CAR comprises a HLA-G-binding portion of an extracellular domain of a LILRB2 protein. In some embodiments, the CAR comprises a portion of a KIR2DL4 intracellular domain, for example a portion of a LILRB2 intracellular domain capable of mediating an activating signal.

Any transmembrane domain may be a transmembrane domain from, for example, CD28, the alpha chain of the T-cell receptor, beta chain of the T-cell receptor, zeta chain of the T-cell receptor, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, GITR/CD357, NKG2D, DAP10, DAP12, a killer immunoglobulin-like receptor (KIR) such as KIR2DL2, LILRB1, LILRB2, or any combination thereof. In some embodiments, the transmembrane domain is a CD28 transmembrane domain. The CD28 transmembrane domain may comprise SEQ ID NO:12. In a specific case, the CD28 transmembrane domain is encoded by SEQ ID NO:11. In some embodiments, the transmembrane domain is a CD8 transmembrane domain. The CD8 transmembrane domain may comprise SEQ ID NO: 14. In a specific case, the CD28 transmembrane domain is encoded by SEQ ID NO: 13.

Any intracellular domain may be an intracellular domain from, for example, CD3 zeta, CD27, CD28, 4-1BB, DAP12, NKG2D, OX-40 (CD134), DAP10, CD40L, 2B4, DNAM, CS1, CD48, NKp30, NKp44, NKp46, or NKp80, or any combination thereof. In some embodiments, the intracellular domain is a CD3 zeta intracellular domain. The CD3 zeta intracellular domain may comprise SEQ ID NO: 16 or SEQ ID NO:18. In a specific case, the CD3 zeta intracellular domain is encoded by SEQ ID NO:15 or SEQ ID NO:17. In some embodiments, the intracellular domain is a CD28 intracellular domain. The CD28 intracellular domain may comprise SEQ ID NO:24. In a specific case, the CD28 intracellular domain is encoded by SEQ ID NO:23. The CAR may comprises two or more, or three or more, intracellular domains. In certain aspect, the two or more intracellular domains comprise a CD3 zeta intracellular domain and an additional intracellular domain selected from a CD28, DAP10, DAP12, 4-1BB, NKG2D, and 2B4 intracellular domain. In a specific case, the two or more intracellular domains comprise a CD3 zeta intracellular domain and a CD28 intracellular domain.

In some embodiments, the CAR further comprises a signal peptide. In certain aspects, the signal peptide is from CD8, CD27, granulocyte-macrophage colony-stimulating factor receptor (GMSCF-R), Ig heavy chain, a KIR (e.g., KIR2DL4), CD3, or CD4. In some embodiments, the signal peptide is a CD8 signal peptide. The CD8 signal peptide may comprise SEQ ID NO:6. In some embodiments, the signal peptide is encoded by SEQ ID NO:5. In certain aspects, the CAR does not comprise a signal peptide.

In certain embodiments, a polynucleotide encoding a CAR of the disclosure further encodes an additional polypeptide of interest. The sequence encoding the additional polypeptide of interest and the sequence encoding the CAR may be separated on the polynucleotide by a 2A element, such as an E2A element. In certain aspects, one or more polypeptides of interest are utilized, such as a therapeutic protein and/or a protein that enhances cell activity, expansion, and/or persistence. In some embodiments, the additional polypeptide(s) of interest is a suicide gene, a cytokine, and/or a human or viral protein that enhances proliferation, expansion and/or metabolic fitness. In certain embodiments the additional polypeptide of interest is a cytokine, for example IL-15, IL-2, IL-12, IL-18, IL-21, IL-23, or IL-7. In a specific embodiment, the cytokine is IL-15.

Aspects of the disclosure are directed to a polypeptide encoding a CAR comprising a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99%, 99.1%, 99.2%, 99.3%, 99.4%, 99.5%, 99.6%, 99.7%, 99.8%, or 99.9% sequence identity with SEQ ID NO:2. In some embodiments, the CAR comprises SEQ ID NO:2. In some aspects, the polynucleotide comprises a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99%, 99.1%, 99.2%, 99.3%, 99.4%, 99.5%, 99.6%, 99.7%, 99.8%, or 99.9% sequence identity with SEQ ID NO:1. In some embodiments, the polynucleotide comprises SEQ ID NO:1.

Additional aspects of the disclosure are directed to a polypeptide encoding a CAR comprising a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99%, 99.1%, 99.2%, 99.3%, 99.4%, 99.5%, 99.6%, 99.7%, 99.8%, or 99.9% sequence identity with SEQ ID NO:4. In some embodiments, the CAR comprises SEQ ID NO:4. In some aspects, the polynucleotide comprises a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99%, 99.1%, 99.2%, 99.3%, 99.4%, 99.5%, 99.6%, 99.7%, 99.8%, or 99.9% sequence identity with SEQ ID NO:3. In some embodiments, the polynucleotide comprises SEQ ID NO:3.

Also presented herein are vectors comprising a polynucleotide of the disclosure. Vectors contemplated herein include viral vectors (e.g., adenoviral vectors, adeno-associated viral vectors, lentiviral vectors, and retroviral vectors) and non-viral vectors (e.g., plasmids).

Embodiments of the disclosure include immune cells of any kind comprising any polynucleotide and/or polypeptide encompassed herein. In specific embodiments, the immune cell is a NK cell, T cell, gamma delta T cell, alpha beta T cell, invariant NKT (INKT) cell, B cell, macrophage, MSC, dendritic cell, or mixture thereof. In cases wherein the immune cell is an NK cell, the NK cell may be derived from cord blood (including pooled cord blood units), peripheral blood, induced pluripotent stem cells, bone marrow, and/or from a cell line. In specific aspects, the NK cell line is NK-92 cell line or another NK cell line derived from a tumor or from a healthy NK cell or a progenitor cell.

In specific embodiments, the immune cell is an NK cell, such as one derived from cord blood, such as from a cord blood mononuclear cell. The NK cell may be a CD56″ NK cell, in specific cases. The NK cells may express one or more exogenously provided cytokines, such as IL-15, IL-2, IL-12, IL-18, IL-21, IL-23, IL-7, or a combination thereof. Particular embodiments include populations of immune cells of any kind of the disclosure, and the cells may be present in a suitable medium or a suitable carrier of any kind.

Methods of treating or preventing cancer of any kind are encompassed herein, including by administering cells expressing particular anti-HLA-G CARs at a therapeutically effective amount to ameliorate or prevent the cancer, or reduce the risk of the cancer, reduce the severity of the cancer, prevent metastasis or risk thereof, or delay the onset of the cancer.

In some embodiments, disclosed is a method of killing HLA-G-positive cells of any kind in an individual comprising administering to the individual an effective amount of cells harboring any polynucleotide and/or polypeptide of the disclosure (e.g., a HLA-G CAR of the disclosure). In specific embodiments, the cells are NK cells, T cells, gamma delta T cells, alpha beta T cells, invariant NKT (INKT) cells, B cells, macrophages, mesenchymal stromal cells (MSCs), or dendritic cells. NK cells may be derived from cord blood, peripheral blood, induced pluripotent stem cells, hematopoietic stem cells, bone marrow, or from a cell line. NK cells may be derived from cord blood mononuclear cells. In some cases, the HLA-G-positive cells are cancer cells, including from hematopoietic cancers or solid tumors. The cells may be allogeneic or autologous with respect to the individual, who may or may not be a human. The cells may be administered to the individual by injection, intravenously, intraarterially, intraperitoneally, intratracheally, intratumorally, intramuscularly, endoscopically, intralesionally, intracranially, percutaneously, subcutaneously, regionally, by perfusion, in a tumor microenvironment, or a combination thereof.

In particular embodiments of the methods, the cells may be administered to the individual once or more than once. The duration of time between administrations of the cells to the individual may be 1-24 hours, 1-7 days, 1-4 weeks, 1-12 months, or 1 or more years. The methods may further comprise the step of providing to the individual an effective amount of an additional therapy, such as surgery, radiation, gene therapy, immunotherapy, and/or hormone therapy. The additional therapy may comprise one or more antibodies or antibody-based agents, in some cases. In some aspects to the methods, they may further comprising the step of identifying HLA-G-positive cells in the individual.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

It is contemplated that any embodiment discussed in this specification can be implemented with respect to any method or composition of the invention, and vice versa. Furthermore, compositions of the invention can be used to achieve methods of the invention.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows expression of HLA-G in various cancer cell types as measured by flow cytometry, including breast cancer (MDAMB231 and BCX010), acute myeloid leukemia (OCI-3), multiple myeloma (MM1), and glioblastoma cells (GCS267, GCS20, GCS8-11).

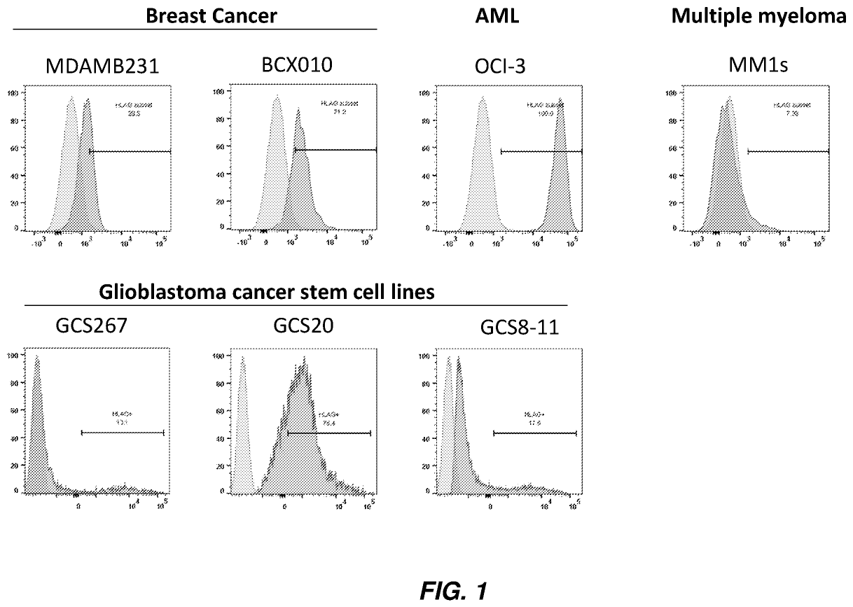

FIG. 2 shows expression of CB-NK cells transduced with an anti-HLA-G CAR of the present disclosure (KIR2DL4 CAR). The KIRD2DL4 CAR peak is the furthest to the right.

FIG. 3 shows results from intracellular cytokine staining of OCI-AML13 cells cultured with KIR2LD4 CAR NK cells of the disclosure (CD8-KIR2DL4-TMD-3Z and CD8-KIR2DL4-CD28-3Z) or non-transduced control NK cells (NT). Increased TNFα, INFγ, and CD107a expression was observed with the KIR2LD4 CAR NK cells compared to the control.

FIG. 4 shows ⁵¹Cr release measured from OCI-AML13 cells cultured with KIR2LD4 CAR NK cells of the disclosure (CD8-KIR2DL4-TMD-3Z and CD8-KIR2DL4-CD28-3Z) or non-transduced control NK cells (NT). Increased Cr release was observed with the KIR2LD4 CAR NK cells compared to the control (bottom line).

FIG. 5 shows HEK293T cell transfection efficiency for an example of a HLA-G CAR construct.

FIG. 6 provides the transduction efficiency for HLA-G CAR NK cells.

FIG. 7 demonstrates that HLA-G CAR cells efficiently kill OCI-AML3 cells.

FIGS. 8A and 8B show that HLA-G CAR NK cells efficiently killing GSC20 spheroids.

FIG. 9 shows that HLA-G CAR NK cells release TNF-α in response to tumor targets.

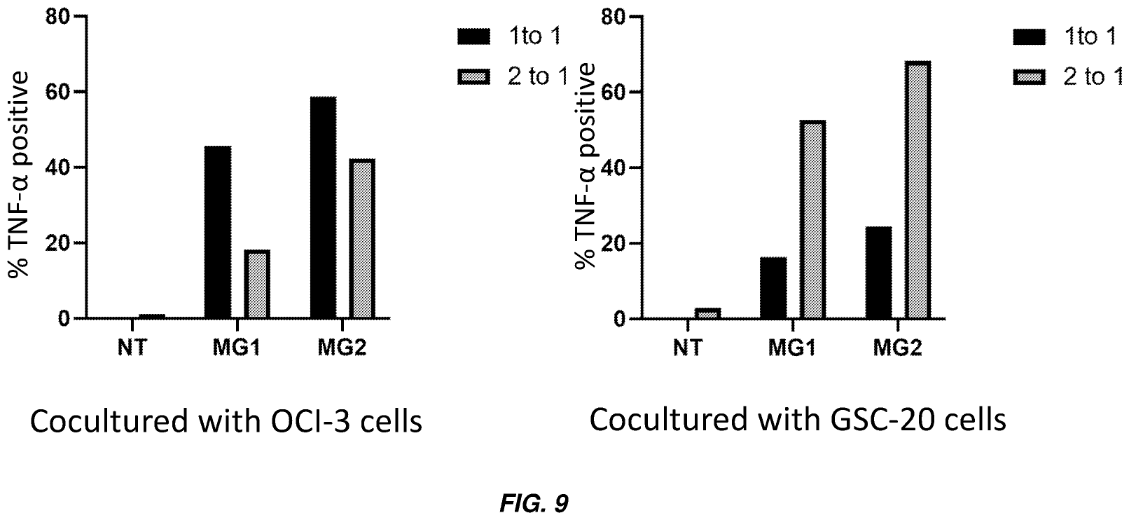

While various embodiments of the disclosure have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the disclosure described herein may be employed.

### DETAILED DESCRIPTION

### I. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure.

The term “isolated” as used herein refers to molecules or biologicals or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, such as that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides that are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

As used herein, “prevent,” and similar words such as “prevented,” “preventing” etc., indicate an approach for preventing, inhibiting, or reducing the likelihood of the occurrence or recurrence of, a disease or condition, e.g., cancer. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

The term “sample,” as used herein, generally refers to a biological sample. The sample may be taken from tissue or cells from an individual. In some examples, the sample may comprise, or be derived from, a tissue biopsy, blood (e.g., whole blood), blood plasma, extracellular fluid, dried blood spots, cultured cells, discarded tissue. The sample may have been isolated from the source prior to collection. Non-limiting examples include blood, cerebral spinal fluid, pleural fluid, amniotic fluid, lymph fluid, saliva, urine, stool, tears, sweat, or mucosal excretions, and other bodily fluids isolated from the primary source prior to collection. In some examples, the sample is isolated from its primary source (cells, tissue, bodily fluids such as blood, environmental samples, etc.) during sample preparation. The sample may or may not be purified or otherwise enriched from its primary source. In some cases the primary source is homogenized prior to further processing. The sample may be filtered or centrifuged to remove buffy coat, lipids, or particulate matter. The sample may also be purified or enriched for nucleic acids, or may be treated with RNases. The sample may contain tissues or cells that are intact, fragmented, or partially degraded.

The term “subject,” as used herein, generally refers to an individual having a biological sample that is undergoing processing or analysis and, in specific cases, has or is suspected of having cancer. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as benign or malignant neoplasias, or cancer. The subject may being undergoing or having undergone treatment. The subject may be asymptomatic. The subject may be healthy individuals but that are desirous of prevention of cancer. The term “individual” may be used interchangeably, in at least some cases. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants and includes in utero individuals. It is not intended that the term connote a need for medical treatment, therefore, an individual may voluntarily or involuntarily be part of experimentation whether clinical or in support of basic science studies.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

The present disclosure concerns methods and compositions directed to therapies for HLA-G-positive cancers, particularly utilizing adoptive cell therapy that targets HLA-G-positive cancer cells. In particular embodiments, genetically engineered mammalian immune cells of any kind (including at least human NK cells) are generated to target HLA-G-positive cancers. The disclosure encompasses a genetically engineered receptor of any kind (including a chimeric antigen receptor (CAR)) that is directed against HLA-G. In specific embodiments there are provided a number of novel expression constructs, including retroviral constructs, that express a HLA-G-targeting extracellular domain (including a KIR2DL4 extracellular domain) used in a CAR and, in some cases, that also express one or more cytokines, such as IL-15, to support cell survival and proliferation. In some embodiments, the CAR is a fusion of the extracellular domain of KIR2DL4 (a natural ligand for HLA-G) and one or more additional domains (e.g., transmembrane domain, intracellular domain) that are not from KIR2DL4.

### II. Genetically Engineered Receptors

The immune cells of the present disclosure can be genetically engineered to express one or more antigen-binding receptors that target HLA-G, such as engineered CARs or, alternatively, engineered TCRs. For example, the immune cells may be immune cells that are modified to express a CAR and/or TCR having antigenic specificity for HLA-G. Other CARS and/or TCRs may be expressed by the same cells as the HLA-G antigen receptor-expressing cells, and they may be directed to different antigens. In some aspects, the immune cells are engineered to express the HLA-G-specific CAR or HLA-G-specific TCR by knock-in of the CAR or TCR using CRISPR/Cas technology.

Suitable methods of modification of cells are known in the art. See, for instance, Sambrook and Ausubel, supra. For example, the cells may be transduced to express a CAR or TCR having antigenic specificity for a cancer antigen using transduction techniques described in Heemskerk et al., 2008 and Johnson et al., 2009.

In some embodiments, the cells comprise one or more nucleic acids introduced via genetic engineering that encode one or more antigen-targeting receptors (at least one of which is directed against HLA-G), and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g., chimeric).

Exemplary antigen receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., 2013; Davila et al., 2013; Turtle et al., 2012; Wu et al., 2012. In some aspects, the genetically engineered antigen receptors include a CAR as described in U.S. Pat. No. 7,446,190, and those described in International Patent Application Publication No.: WO/2014055668 A1.

### A. Chimeric Antigen Receptors

In particular embodiments, a HLA-G-specific CAR is utilized that comprises at least: a) one or more intracellular signaling domains, b) a transmembrane domain, and c) an extracellular domain comprising at least one antigen binding region that targets, including specifically binds, HLA-G. In some embodiments the antigen binding region is an antibody or functional fragment thereof. In other cases the antigen binding region of the CAR is not an antibody or functional fragment thereof (such as a ligand for HLA-G such as KIR2DL4, LILRB1, or LILRB2). In some embodiments, the antigen binding region of the CAR is a KIR2DL4 extracellular domain, or antigen binding portion thereof. In some embodiments, the antigen binding region of the CAR is a LILRB1 extracellular domain, or antigen binding portion thereof. In some embodiments, the antigen binding region of the CAR is a LILRB2 extracellular domain, or antigen binding portion thereof. In some embodiments, the HLA-G-specific CAR binds only HLA-G, whereas in other cases the CAR as a single polypeptide is bispecific by comprising two or more antigen binding domains, one of which that binds HLA-G and the other of which binds another, non-identical antigen.

In some embodiments, the engineered antigen receptors include CARs, including activating or stimulatory CARs, or costimulatory CARs (see WO2014/055668. The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

It is contemplated that the chimeric construct can be introduced into immune cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric CAR construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV.

Certain embodiments of the present disclosure concern the use of nucleic acids, including nucleic acids encoding a HLA-G-specific CAR polypeptide, including in some cases a CAR that has been humanized to reduce immunogenicity (hCAR), comprising at least one intracellular signaling domain, a transmembrane domain, and an extracellular domain comprising one or more signaling motifs. In certain embodiments, the HLA-G-specific CAR may recognize an epitope comprising the shared space between one or more antigens. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor.

It is contemplated that the human HLA-G CAR nucleic acids may be human genes used to enhance cellular immunotherapy for human patients. In a specific embodiment, the disclosure includes a full-length HLA-G-specific CAR cDNA or coding region. The antigen binding regions or domain can comprise a fragment of the V_{H} and V_{L} chains of a single-chain variable fragment (scFv) derived from a particular human monoclonal antibody, such as those described in U.S. Pat. No. 7,109,304, incorporated herein by reference. The fragment can also be any number of different antigen binding domains of a human antigen-specific antibody. In a more specific embodiment, the fragment is a HLA-G-specific scFv encoded by a sequence that is optimized for human codon usage for expression in human cells.

The arrangement could be multimeric, such as a diabody or multimers. The multimers are most likely formed by cross pairing of the variable portion of the light and heavy chains into a diabody. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One could use just one of the Fc domains, e.g., either the CH2 or CH3 domain from human immunoglobulin. One could also use the hinge, CH2 and CH3 region of a human immunoglobulin that has been modified to improve dimerization. One could also use just the hinge portion of an immunoglobulin. One could also use portions of CD8alpha.

In some embodiments, HLA-G-specific CAR is constructed with specificity for HLA-G, such as HLA-G being expressed on a diseased cell type. Thus, the CAR typically includes in its extracellular portion one or more HLA-G-binding molecules, such as one or more antigen-binding fragments, domains, antibody variable domains, and/or antibody molecules of any kind. An example of a human HLA-G nucleic acid is at National Center for Biotechnology Information's GenBank® database at Accession No. NM_002127. An example of a human HLA-G polypeptide is at GenBank® Accession No. NP_002118. One of skill in the art is able to generate antibodies, including scFvs against HLA-G based on knowledge at least of the polypeptide and routine practices, although numerous anti-HLA-G scFvs and monoclonal antibodies are already present in the art. In some embodiments, the HLA-G-specific scFv is an scFV from one or more of antibody clones.

In some embodiments, the HLA-G-specific CAR includes an antigen-binding portion or portions of an antibody molecule, such as a single-chain antibody fragment (scFv) derived from the variable heavy (VH) and variable light (VL) chains of a monoclonal antibody (mAb). In specific embodiments, the antibody or functional fragment thereof is or is derived from G233, 26-2H11, MEM-G/1, MEM-G/9, MEM-G/11, MEM-G/13, 1B8, 5E6H7, 1-2C3, 16G1, 5A6G7, 87G, or 3C/G4. The antibody may also be one that is generated de novo against HLA-G, and the scFv sequence may be obtained, or derived, from such de novo antibodies.

In certain embodiments, the anti-HLA-G CAR comprises an extracellular domain that is or comprises a ligand for HLA-G. In specific embodiments, the anti-HLA-G CAR comprises an extracellular domain from KIR2DL4, LILRB1, LILR2, or fragments or mimetics thereof. In some embodiments, the anti-HLA-G CAR comprises a KIR2DL4 extracellular domain or antigen binding portion thereof. In some embodiments, the anti-HLA-G CAR comprises an extracellular domain comprising SEQ ID NO:4.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof, as it is found that introns stabilize the mRNA. Also, it may be further advantageous to use endogenous or exogenous non-coding regions to stabilize the mRNA.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex. The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, DAP12, DAP10, NKG2D, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, a KIR such as KIR2DL4, GITR/CD357, and so forth. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

In some embodiments, the HLA-G CAR nucleic acid comprises a sequence encoding other costimulatory receptors, such as a transmembrane domain and one or more intracellular signaling domains. In addition to a primary T cell activation signal, such as may be initiated by CD3ζ and/or FcεRIγ, an additional stimulatory signal for immune effector cell proliferation and effector function following engagement of the chimeric receptor with the target antigen may be utilized. For example, part or all of a human costimulatory receptor for enhanced activation of cells may be utilized that could help improve in vivo persistence and improve the therapeutic success of the adoptive immunotherapy. Examples include costimulatory domains from molecules such as DAP12, DAP10, NKG2D, CD2, CD28, CD27, 4-1BB, (CD137), OX40, ICOS, (CD278), CD30, HVEM, CD40, LFA-1 (CD11a/CD18), ICAM-1, and/or a portion of a KIR2DL4 cytoplasmic domain capable of inducing an activating signal, although in specific alternative embodiments any one of these listed may be excluded from use in the CAR.

In certain embodiments, the platform technologies disclosed herein to genetically modify immune cells, such as NK cells, comprise (i) non-viral gene transfer using an electroporation device (e.g., a nucleofector), (ii) CARs that signal through endodomains (e.g., CD28/CD3-ζ, CD137/CD3-ζ, or other combinations), (iii) CARs with variable lengths of extracellular domains connecting the HLA-G-recognition domain to the cell surface, and, in some cases, (iv) artificial antigen presenting cells (aAPC) derived from K562 to be able to robustly and numerically expand CAR″ immune cells (Singh et al., 2008; Singh et al., 2011).

### B. Examples of Specific CAR Embodiments

In particular embodiments, specific HLA-G CAR molecules are encompassed herein. In some cases, the HLA-G binding domain of the CAR is a scFv, and any scFv that binds to HLA-G may be utilized herein. In some cases, the HLA-G binding domain of the CAR is a binding domain from a ligand of HLA-G (e.g., KIR2DL4, LILRB1, or LILRB2), and any domain that binds HLA-G may be utilized herein. In cases wherein an anti-HLA-G scFv is utilized in the extracellular domain of the CAR, the variable heavy chain and the variable light chain for the scFv may be in any order in N-terminal to C-terminal direction. For example, the variable heavy chain may be on the N-terminal side of the variable light chain, or vice versa. The scFv and/or ligand that binds HLA-G in the CAR may or may not be codon optimized. In particular embodiments, a vector encodes a HLA-G-specific CAR and also encodes one or more other molecules. For example, a vector may encode a HLA-G-specific CAR and also may encode another protein of interest, such as another engineered antigen receptor, a suicide gene, and/or a particular cytokine.

On the same molecule, the HLA-G-specific CAR may comprise one or more antigen-specific extracellular domains, a specific hinge, a specific transmembrane domain, one or more specific costimulatory domains, and one or more specific activation signals. When more than one antigen-specific extracellular domain is utilized, such as for targeting two different antigens (one of which is HLA-G), there may be a linker between the two antigen-specific extracellular domains.

In particular embodiments of specific CAR molecules, a CAR may utilize DAP10, DAP12, 4-1BB, NKG2D, or other costimulatory domains (which may be referred to herein as an intracytoplasmic domain). In some cases, CD3zeta is utilized without any costimulatory domains. In particular embodiments of specific CAR molecules, a CAR may utilize any suitable transmembrane domain, such as from DAP12, DAP10, 4-1BB, 2B4, OX40, CD27, NKG2D, CD8, or CD28.

In particular embodiments, there is an expression construct comprising sequence that encodes a particular HLA-G-specific engineered receptor. In particular embodiments, any HLA-G CAR may comprise one of the following: (a) CD8 signal peptide, KIR2DL4 extracellular domain, CD28 transmembrane domain, CD3zeta intracellular domain; or (b) CD8 signal peptide, KIR2DL4 extracellular domain, CD8 transmembrane domain, CD28 intracellular domain, and CD3zeta intracellular domain.

Examples of specific sequence embodiments are provided below.

### 1. Antigen-Specific Extracellular Domains

In specific embodiments, an antigen-specific extracellular domain comprises a KIR2DL4 extracellular domain and/or an anti-HLA-G antigen binding region of an HLA-G specific antibody. Examples of specific sequence embodiments are provided below.

In specific embodiments, a KIR2DL4 extracellular domain nucleotide sequence is utilized, as follows:

(SEQ ID NO: 7)
TGGGCACACGTGGGTGGTCAGGACAAGCCCTTCTGCTCTGCCTGG
CCCAGCGCTGTGGTGCCTCAAGGAGGACACGTGACTCTTCGGTGT
CACTATCGTCGTGGGTTCAACATCTTCACGCTGTACAAGAAAGAT
GGGGTCCCTGTCCCTGAGCTCTACAACAGAATATTCTGGAACAGT
TTCCTCATTAGCCCTGTGACCCCAGCACACGCAGGGACCTACAGA
TGTCGAGGTTTTCACCCGCACTCCCCCACTGAGTGGTCGGCACCC
AGCAACCCCCTGGTGATCATGGTCACAGGTCTATATGAGAAACCT
TCGCTTACAGCCCGGCTGGGCCCCACGGTTCGCGCAGGAGAGAAC
GTGACCTTGTCCTGCAGCTCCCAGAGCTCCTTTGACATCTACCAT
CTATCCAGGGAGGGGGAAGCCCATGAACTTAGGCTCCCTGCAGTG
CCCAGCATCAATGGAACATTCCAGGCCGACTTCCCTCTGGGTCCT
GCCACCCACGGAGAGACCTACAGATGCTTCGGCTCTTTCCATGGA
TCTCCCTACGAGTGGTCAGACCCGAGTGACCCACTGCCTGTTTCT
GTCACAGGAAACCCTTCTAGTAGTTGGCCTTCACCCACTGAACCA
AGCTTCAAAACTGGTATCGCCAGACACCTGCAT

Any polynucleotide encompassed by the present disclosure may comprise SEQ ID NO:7 or a sequence that is at least 70, 75, 80, 85, 90, 95, 96, 97, 98, 99, or more % identical to SEQ ID NO:7.

In certain embodiments, a codon optimized KIR2DL4 extracellular domain nucleotide sequence is utilized, as follows:

(SEQ ID NO: 8)
TGGGCTCACGTTGGCGGCCAGGATAAGCCTTTTTGTTCTGCCTGG
CCTAGCGCCGTGGTTCCTCAAGGTGGACACGTGACCCTGCGGTGT
CACTACAGACGGGGCTTCAACATCTTCACCCTGTACAAGAAAGAC
GGCGTGCCCGTGCCTGAGCTGTACAACAGAATCTTCTGGAACAGC
TTCCTGATCAGCCCCGTGACACCAGCTCACGCCGGCACATACAGA
TGCAGAGGCTTTCACCCTCACAGCCCCACAGAGTGGTCCGCTCCA
TCTAACCCTCTGGTCATCATGGTCACCGGCCTGTACGAGAAGCCT
AGCCTGACAGCTAGACTGGGCCCTACAGTTAGAGCCGGCGAGAAT
GTGACCCTGTCCTGTAGCAGCCAGAGCAGCTTCGACATCTACCAC
CTGTCTAGAGAGGGCGAAGCCCACGAACTGAGACTGCCTGCCGTG
CCTAGCATCAATGGCACCTTCCAGGCCGATTTTCCACTGGGACCT
GCCACACACGGCGAGACTTACAGATGCTTTGGCAGCTTCCACGGC
AGCCCTTACGAGTGGTCTGATCCTAGCGATCCTCTGCCTGTGTCC
GTGACAGGCAATCCTAGCAGCAGCTGGCCTTCTCCAACCGAGCCT
AGCTTTAAGACCGGAATCGCCCGGCATCTGCAC

Any polynucleotide encompassed by the present disclosure may comprise SEQ ID NO:8 or a sequence that is at least 70, 75, 80, 85, 90, 95, 96, 97, 98, 99, or more % identical to SEQ ID NO:8.

An example KIR2DL4 extracellular domain amino acid sequence is as follows:

(SEQ ID NO: 9)
WAHVGGQDKPFCSAWPSAVVPQGGHVTLRCHYRRGFNIFTLYKKD
GVPVPELYNRIFWNSFLISPVTPAHAGTYRCRGFHPHSPTEWSAP
SNPLVIMVTGLYEKPSLTARLGPTVRAGENVTLSCSSQSSFDIYH
LSREGEAHELRLPAVPSINGTFQADFPLGPATHGETYRCFGSFHG
SPYEWSDPSDPLPVSVTGNPSSSWPSPTEPSFKTGIARHLH

Any polypeptide encompassed by the present disclosure may comprise SEQ ID NO:9 or a sequence that is at least 70, 75, 80, 85, 90, 95, 96, 97, 98, 99, or more % identical to SEQ ID NO:9.

An additional example KIR2DL4 extracellular domain amino acid sequence is as follows:

(SEQ ID NO: 10)
WAHVGGQDKPFCSAWPSAVVPQGGHVTLRCHYRRGFNIFTLYKKD
GVPVPELYNRIFWNSFLISPVTPAHAGTYRCRGFHPHSPTEWSAP
SNPLVIMVTGLYEKPSLTARPGPTVRAGENVTLSCSSQSSFDIYH
LSREGEAHELRLPAVPSINGTFQADFPLGPATHGETYRCFGSFHG
SPYEWSDPSDPLPVSVTGNPSSSWPSPTEPSFKTGIARHLH

Any polypeptide encompassed by the present disclosure may comprise SEQ ID NO:9 or a sequence that is at least 70, 75, 80, 85, 90, 95, 96, 97, 98, 99, or more % identical to SEQ ID NO:9.

In specific examples, the region of KIR2DL4 that is utilized in the CAR molecule comprises, consists of, or consists essentially of amino acids 1-50, 1-51, 1-52, 1-53, 1-54, 1-55, 1-56, 1-57, 1-58, 1-59, 1-60, 1-61, 1-62, 1-63, 1-64, 1-65, 1-66, 1-67, 1-68, 1-69, 1-70, 1-71, 1-72, 1-73, 1-74, 1-75, 1-76, 1-77, 1-78, 1-79, 1-80, 1-81, 1-82, 1-83, 1-84, 1-85, 1-86, 1-87, 1-88, 1-89, 1-90, 1-91, 1-92, 1-93, 1-94, 1-95, 1-96, 1-97, 1-98, 1-99, 1-100, 1-101, 1-102, 1-103, 1-104, 1-105, 1-106, 1-107, 1-108, 1-109, 1-110, 1-111, 1-112, 1-113, 1-114, 1-115, 1-116, 1-117, 1-118, 1-119, 1-120, 1-121, 1-122, 1-123, 1-124, 1-125, 1-126, 1-127, 1-128, 1-129, 1-130, 1-131, 1-132, 1-133, 1-134, 1-135, 1-136, 1-137, 1-138, 1-139, 1-140, 1-141, 1-142, 1-143, 1-144, 1-145, 1-146, 1-147, 1-148, 1-149, 1-150, 1-151, 1-152, 1-153, 1-154, 1-155, 1-156, 1-157, 1-158, 1-159, 1-160, 1-161, 1-162, 1-163, 1-164, 1-165, 1-166, 1-167, 1-168, 1-169, 1-170, 1-171, 1-172, 1-173, 1-174, 1-175, 1-176, 1-177, 1-178, 1-179, 1-180, 1-181, 1-182, 1-183, 1-184, 1-185, 1-186, 1-187, 1-188, 1-189, 1-190, 1-191, 1-192, 1-193, 1-194, 1-195, 1-196, 1-197, 1-198, 1-199, 1-200, 1-201, 1-202, 1-203, 1-204, 1-205, 1-206, 1-207, 1-208, 1-209, 1-210, 1-211, 1-212, 1-213, 1-214, 1-215, 1-216, 1-217, 1-218, 1-219, 1-220, or all (1-221) of SEQ ID NO:9 or SEQ ID NO:10; in specific embodiments, such amino acids in these ranges are contiguous. In some embodiments, a region of SEQ ID NO:9 or SEQ ID NO:10 is utilized that has truncation at the N-terminus, such as 1, 2, 3, 4, 5, 6, 7, 8, 9, 10, 11, 12, 13, 14, 15, 16, 17, 18, 19, 20, or more amino acids from the N-terminus. In certain cases, there is truncation at that N-terminus of 1, 2, 3, 4, 5, 6, 7, 8, 9, 10, 11, 12, 13, 14, 15, 16, 17, 18, 19, 20, or more amino acids and there is truncation at the C-terminus.

An LILRB1 extracellular domain amino acid sequence is as follows:

(SEQ ID NO: 48)
GHLPKPTLWAEPGSVITQGSPVTLRCQGGQETQEYRLYREKKTAL
WITRIPQELVKKGQFPIPSITWEHAGRYRCYYGSDTAGRSESSDP
LELVVTGAYIKPTLSAQPSPVVNSGGNVILQCDSQVAFDGFSLCK
EGEDEHPQCLNSQPHARGSSRAIFSVGPVSPSRRWWYRCYAYDSN
SPYEWSLPSDLLELLVLGVSKKPSLSVQPGPIVAPEETLTLQCGS
DAGYNRFVLYKDGERDFLQLAGAQPQAGLSQANFTLGPVSRSYGG
QYRCYGAHNLSSEWSAPSDPLDILIAGQFYDRVSLSVQPGPTVAS
GENVTLLCQSQGWMQTFLLTKEGAADDPWRLRSTYQSQKYQAEFP
MGPVTSAHAGTYRCYGSQSSKPYLLTHPSDPLELVVSGPSGGPSS
PTTGPTSTSGPEDQPLTPTGSDPQSGLGRHLGV

An LILRB2 extracellular domain amino acid sequence is as follows:

(SEQ ID NO: 49)
QTGTIPKPTLWAEPDSVITQGSPVTLSCQGSLEAQEYRLYREKKS
ASWITRIRPELVKNGQFHIPSITWEHTGRYGCQYYSRARWSELSD
PLVLVMTGAYPKPTLSAQPSPVVTSGGRVTLQCESQVAFGGFILC
KEGEEEHPQCLNSQPHARGSSRAIFSVGPVSPNRRWSHRCYGYDY
DRFVLYKEGERDLRQLPGRQPQAGLSQANFTLGPVSRSYGGQYRC
YGAHNLSSECSAPSDPLDILITGQIRGTPFISVQPGPTVASGENV
TLLCQSWRQFHTFLLTKAGAADAPLRLRSIHEYPKYQAEFPMSPV
TSAHAGTYRCYGSLNSDPYLLSHPSEPLELVVSGPSMGSSPPPTG
PISTPAGPEDQPLTPTGSDPQSGLGRHLGV

In specific embodiments, the antigen-specific extracellular domain comprises an anti-HLA-G antigen binding region of an HLA-G specific antibody, which may or may not be an scFv.

In specific embodiments, the antigen-specific extracellular domain comprises an antibody from a HLA-G monoclonal antibody from MEM-G11. In such cases, the MEM-G/11 sequence may comprise the following light chain sequence including variable and constant regions:

(SEQ ID NO: 50)
DIVLTQSPASLDVSLGQRATISCRASKSVSTSGYSYMHWYQQKPG
QSPKLLIYLASNRESGVPARFSGSGSGTDFTLNIHPVEEEDAATY
YCQHSREFPTFGAGTKLELKRADAAPTVSIFPPSSEQLTSGGASV
VCFLNNFYPKDINVKWKIDGSERQNGVLNSWTDQDSKDSTYSMSS
TLTLTKDEYERHNSYTCEATHKTSTSPIVKSFNRNEC,

where the constant region is underlined. In specific embodiments, such a light chain sequence may comprise CDR1 of RASKSVSTSGYSYMH (SEQ ID NO:51); a CDR2 of LASNRES (SEQ ID NO:52); and a CDR3 of QHSREFPT (SEQ ID NO:53).

The MEM-G/11 variable light chain sequence may comprise:

(SEQ ID NO: 54)
DIVLTQSPASLDVSLGQRATISCRASKSVSTSGYSYMHWYQQKPG
QSPKLLIYLASNRESGVPARFSGSGSGTDFTLNIHPVEEEDAATY
YCQHSREFPTFGAGTKLELK.

In specific cases, a polynucleotide that encodes the MEM-G/11 variable light chain sequence may comprise:

(SEQ ID NO: 55)
Gatattgtgctgacccagagcccggcgagcctggatgtgagcctg
ggccagcgcgcgaccattagctgccgcgcgagcaaaagcgtgagc
accagcggctatagctatatgcattggtatcagcagaaaccgggc
cagagcccgaaactgctgatttatctggcgagcaaccgcgaaagc
ggcgtgccggcgcgctttagcggcagcggcagcggcaccgatttt
accctgaacattcatccggtggaagaagaagatgcggcgacctat
tattgccagcatagccgcgaatttccgacctttggcgcgggcacc
aaactggaactgaaa

In specific cases, the MEM-G/11 sequence may comprise the following heavy chain sequence including variable and constant regions:

(SEQ ID NO: 56)
KVQLVESGGGLVKPGGSLKLSCSASGFPFSDYYMYWVRQTPEKRL
EWVATISDDDDYTYYPDSMKGRFTISRDNAKNNLYLQMSSLKSED
TAMYYCSRGIYYGSSPFAYWGQGTLVTVSAAKTTPPSVYPLAPGS
AAQTNSMVTLGCLVKGYFPEPVTVTWNSGSLSSGVHTFPAVLQSD
LYTLSSSVTVPSSTWPSETVTCNVAHPASSTKVDKKIVPRDCGCK
PCICTVPEVSSVFIFPPKPKDVLTITLTPKVTCVVVDISKDDPEV
QFSWFVDDVEVHTAQTQPREEQFNSTFRSVSELPIMHQDWLNGKE
FKCRVNSAAFPAPIEKTISKTKGRPKAPQVYTIPPPKEQMAKDKV
SLTCMITDFFPEDITVEWQWNGQPAENYKNTQPIMDTDGSYFVYS
KLNVQKSNWEAGNTFTCSVLHEGLHNHHTEKSLSHSPGK,

wherein the constant region is underlined. In specific embodiments, such a heavy chain sequence may comprise CDR1 of GFPFSDY (SEQ ID NO:57); a CDR2 of SDDDDY (SEQ ID NO:58) and a CDR3 of GIYYGSSPFAY (SEQ ID NO:59).

The MEM-G/11 variable heavy chain sequence may comprise:

(SEQ ID NO: 60)
KVQLVESGGGLVKPGGSLKLSCSASGFPFSDYYMYWVRQTPEKRL
EWVATISDDDDYTYYPDSMKGRFTISRDNAKNNLYLQMSSLKSED
TAMYYCSRGIYYGSSPFAYWGQGTLVTVSA.

In specific cases, a polynucleotide that encodes the MEM-G/11 variable heavy chain sequence may comprise:

(SEQ ID NO: 61)
Aaagtgcagctggtggaaagcggcggcggcctggtgaaaccgggg
gcagcctgaaactgagctgcagcgcgagcggctttccgtttagcg
attattatatgtattgggtgcgccagaccccggaaaaacgcctgg
aatgggtggcgaccattagcgatgatgatgattatacctattatc
cggatagcatgaaaggccgctttaccattagccgcgataacgcga
aaaacaacctgtatctgcagatgagcagcctgaaaagcgaagata
ccgcgatgtattattgcagccgcggcatttattatggcagcagcc
cgtttgcgtattggggccagggcaccctggtgaccgtgagcgcg

In specific embodiments, the antigen-specific extracellular domain comprises an antibody from a HLA-G monoclonal antibody from 87G clone. In such cases, the sequence may comprise the following light chain sequence that includes variable and constant regions:

(SEQ ID NO: 62)
ETTVTQSPASLSVATGEKVTIRCITSTDIDDDMNWYQQKPGEPPK
LLISEDNILRPGVPSRFSSSGYGTDFVFTIENTLSEDVADYYCLQ
SDNMPLTFGGGTRLEIKRADAAPTVSIFPPSSEQLTSGGASVVCF
LNNFYPKDINVKWKIDGSERQNGVLNSWTDQDSKDSTYSMSSTLT
LTKDEYERHNSYTCEATHKTSTSPIVKSFNRNEC,

where the constant region is underlined. In specific embodiments, such a light chain sequence may comprise CDR1 of ITSTDIDDDMN (SEQ ID NO:63); a CDR2 of EDNILRP (SEQ ID NO:64); and a CDR3 of LQSDNMPLT (SEQ ID NO:65).

The 87G variable light chain sequence may comprise:

(SEQ ID NO: 66)
ETTVTQSPASLSVATGEKVTIRCITSTDIDDDMNWYQQKPGEPPK
LLISEDNILRPGVPSRFSSSGYGTDFVFTIENTLSEDVADYYCLQ
SDNMPLTFGGGTRLEIK.

A polynucleotide that may encode the 87G variable light chain sequence may comprise:

(SEQ ID NO: 67)
Gaaaccaccgtgacccagagcccggcgagcctgagcgtggcgacc
ggcgaaaaagtgaccattcgctgcattaccagcaccgatattgat
gatgatatgaactggtatcagcagaaaccgggcgaaccgccgaaa
ctgctgattagcgaagataacattctgcgcccgggcgtgccgagc
cgctttagcagcagcggctatggcaccgattttgtgtttaccatt
gaaaacaccctgagcgaagatgtggcggattattattgcctgcag
agcgataacatgccgctgacctttggcggeggcacccgcctggaa
attaaa.

In specific cases, a sequence may comprise the following heavy chain sequence including variable and constant regions:

(SEQ ID NO: 68)
EVKLVESGGSLVQPGGSLKLSCAASGFSFSSYTMSWVRQTPKKRL
EWVAYVSNGAGTTYYPDSLKGRFTISRDNAKNTLHLLMTSLKSED
TAIYYCARHYYGSYHFDYWGQGTTLIVSSAKTTAPSVYPLAPVCG
GTTGSSVTLGCLVKGYFPEPVTLTWNSGSLSSGVHTFPALLQSGL
YTLSSSVTVTSNTWPSQTITCNVAHPASSTKVDKKIEPRVPITQN
PCPPLKECPPCAAPDLLGGPSVFIFPPKIKDVLMISLSPMVTCVV
VDVSEDDPDVQISWFVNNVEVHTAQTQTHREDYNSTLRVVSALPI
QHQDWMSGKEFKCKVNNRALPSPIEKTISKPRGPVRAPQVYVLPP
PAEEMTKKEFSLTCMITGFLPAEIAVDWTSNGRTEQNYKNTATVL
DSDGSYFMYSKLRVQKSTWERGSLFACSVVHEGLHNHLTTKTISR
SLGK,

where the constant region is underlined. In specific embodiments, such a heavy chain sequence may comprise CDR1 of GFSFSSY (SEQ ID NO:69); a CDR2 of SNGAGT (SEQ ID NO:70); and a CDR3 of HYYGSYHFDY (SEQ ID NO:71).

In some embodiments, the 87G variable heavy chain sequence comprises:

(SEQ ID NO: 72)
EVKLVESGGSLVQPGGSLKLSCAASGFSFSSYTMSWVRQTPKKRL
EWVAYVSNGAGTTYYPDSLKGRFTISRDNAKNTLHLLMTSLKSED
TAIYYCARHYYGSYHFDYWGQGTTLIVSS

In specific cases, a polynucleotide that encodes the 87G variable heavy chain sequence comprises:

(SEQ ID NO: 73)
Gaagtgaaactggtggaaagcggcggcagcctggtgcagccgggc
ggcagcctgaaactgagctgcgcggcgagcggctttagctttagc
agctataccatgagctgggtgcgccagaccccgaaaaaacgcctg
gaatgggtggcgtatgtgagcaacggcgcgggcaccacctattat
ccggatagcctgaaaggccgctttaccattagccgcgataacgcg
aaaaacaccctgcatctgctgatgaccagcctgaaaagcgaagat
accgcgatttattattgcgcgcgccattattatggcagctatcat
tttgattattggggccagggcaccaccctgattgtgagcagc

Any polynucleotide encompassed by the present disclosure may comprise SEQ ID NO:55, 61, 67, or 73, or a sequence that is at least 70, 75, 80, 85, 90, 95, 96, 97, 98, 99, or more % identical to SEQ ID NO: 55, 61, 67, or 73, respectively.

Any polypeptide encompassed by the present disclosure may comprise SEQ ID NO:50-54, 56-60, 62-66, or 68-72, or a sequence that is at least 70, 75, 80, 85, 90, 95, 96, 97, 98, 99, or more % identical to SEQ ID NO: 50-54, 56-60, 62-66, or 68-72, respectively.

### 2. Transmembrane Domains

Any suitable transmembrane domain may be utilized in a HLA-G-specific CAR of the disclosure. Examples include at least transmembrane domains from DAP10, DAP12, CD28, NKG2D, CD3 epsilon, CD4, CD5, CD8, CD9, CD16, CD22, CD28, CD33, CD37, CD45, CD64, CD80, CD86, CD134, CD137, or CD154, from a T-cell receptor a or b chain, from a CD3 zeta chain, from ICOS, functional derivatives thereof, and combinations thereof. In specific cases, a transmembrane domain from DAP10, DAP12, CD28, CD8, or NKG2D is utilized. In some embodiments, a transmembrane domain from a killer immunoglobulin-like receptor (KIR) is utilized, for example a transmembrane domain from an inhibitory KIR (e.g., KIR2DL1, KIR2DL2, KIR2DL3, KIR2DL4, KIR3DL1, KIR3DL2, KIR3DL3) or from an activating KIR (e.g., KIR2DS1, KIR2DS2, KIR2DS3, KIR2DS4, KIR2DS5, KIR3DS1). Examples of particular transmembrane domain sequences may be used, as follows:

CD28 transmembrane domain nucleotide sequence:

(SEQ ID NO: 11)
TTTTGGGTGCTGGTGGTGGTTGGTGGAGTCCTGGCTTGCTATAGC
TTGCTAGTAACAGTGGCCTTTATTATTTTCTGGGTG

CD28 transmembrane domain amino acid sequence:

(SEQ ID NO: 12)
FWVLVVVGGVLACYSLLVTVAFIIFWV

CD8 transmembrane domain nucleotide sequence:

(SEQ ID NO: 13)
ACCACAACACCAGCACCTAGACCTCCAACTCCAGCTCCTACAATC
GCCAGCCAGCCTCTGTCTCTGAGGCCTGAAGCTTGTAGACCTGCT
GCTGGCGGAGCCGTGCATACCAGAGGACTGGATTTCGCCTGCGAT
ATCTACATCTGGGCCCCTCTGGCTGGAACATGTGGCGTGCTGCTG
CTGAGCCTCGTGATCACA

CD8 transmembrane domain amino acid sequence:

(SEQ ID NO: 14)
TTTPAPRPPTPAPTIASQPLSLRPEACRPAAGGA
VHTRGLDFACDIYIWAPLAGTCGVLLLSLVIT

4-1BB transmembrane domain nucleotide sequence:

(SEQ ID NO: 25)
ATCATCTCCTTCTTTCTTGCGCTGACGTCGACTGCGTTGCT
CTTCCTGCTGTTCTTCCTCACGCTCCGTTTCTCTGTTGTT

4-1BB transmembrane domain amino acid sequence:

(SEQ ID NO: 26)
IISFFLALTSTALLFLLFFLTLRFSVV

DAP10 transmembrane domain nucleotide sequence:

(SEQ ID NO: 27)
CTCCTGGCAGGCCTCGTGGCTGCTGATGCGG
TGGCATCGCTGCTCATCGTGGGGGCGGTGTTC

DAP10 transmembrane domain amino acid sequence:

(SEQ ID NO: 28)
LLAGLVAADAVASLLIVGAVF

DAP12 transmembrane domain nucleotide sequence:

(SEQ ID NO: 29)
GGCGTGCTGGCAGGGATCGTGATGGGAGACCT
GGTGCTGACAGTGCTCATTGCCCTGGCCGTG

DAP12 transmembrane domain amino acid sequence:

(SEQ ID NO: 30)
GVLAGIVMGDLVLTVLIALAV

NKG2D transmembrane domain nucleotide sequence:

(SEQ ID NO: 31)
GCGGTGATGATTATTTTTCGCATTGGCATGGC
GGTGGCGATTTTTTGCTGCTTTTTTTTTCCG

NKG2D transmembrane domain amino acid sequence:

(SEQ ID NO: 32)
AVMIIFRIGMAVAIFCCFFFP

Any polynucleotide encompassed by the present disclosure may comprise SEQ ID NO:11, 13, 25, 27, 29, or 31 or a sequence that is at least 70, 75, 80, 85, 90, 95, 96, 97, 98, 99, or more % identical to SEQ ID NO: 11, 13, 25, 27, 29, or 31. Any polypeptide encompassed by the present disclosure may comprise SEQ ID NO:12, 14, 16, 28, 30, or 32 or a sequence that is at least 70, 75, 80, 85, 90, 95, 96, 97, 98, 99, or more % identical to SEQ ID NO: 12, 14, 16, 28, 30, or 32.

### 3. Intracellular Domains

One or more intracellular domains (which may also be referred to herein as signal activation domains or costimulatory domains, in appropriate cases) may or may not be utilized in specific anti-HLA-G CARs of the disclosure. Specific examples include intracellular domains from CD3 zeta, 4-1BB, NKG2D, OX-40, CD27, DAP10, DAP12, B7-1/CD80, CD28, 2B4, 4-1BBL, B7-2/CD86, CTLA-4, B7-H1/PD-L1, ICOS, B7-H2, PD-1, B7-H3, PD-L2, B7-H4, PDCD6, BTLA, or a combination thereof. In some embodiments, an intracellular domain of a CAR of the disclosure is not a KIR2DL4 intracellular domain.

Examples of particular intracellular domains which may be used in a CAR of the disclosure are as follows:

An example CD3zeta intracellular domain nucleotide sequence:

(SEQ ID NO: 15)
ACGCGTAAGAAGTTCAGCAGGAGCGCAGACGCCCCCGCGTACCAG
CAGGGCCAGAACCAGCTCTATAACGAGCTCAATCTAGGACGAAGA
GAGGAGTACGATGTTTTGGACAAAAGACGTGGCCGGGACCCTGAG
ATGGGGGGAAAGCCGAGAAGGAAGAACCCTCAGGAAGGCCTGTAC
AATGAACTGCAGAAAGATAAGATGGCGGAGGCCTACAGTGAGATT
GGGATGAAAGGCGAGCGCCGGAGGGGCAAGGGGCACGATGGCCTT
TACCAGGGTCTCAGTACAGCCACCAAGGACACCTACGACGCCCTT
CACATGCAGGCCCTGCCCCCTCGCGGA

An example CD3zeta intracellular domain amino acid sequence:

(SEQ ID NO: 16)
TRKKFSRSADAPAYQQGQNQLYNELNLGRREEYDVLDKRRGRDPE
MGGKPRRKNPQEGLYNELQKDKMAEAYSEIGMKGERRRGKGHDGL
YQGLSTATKDTYDALHMQALPPRG

An example CD3zeta intracellular domain nucleotide sequence:

(SEQ ID NO: 17)
AAACGCGTGAAGTTCAGCAGGAGCGCAGACGCCCCCGCGTACCAG
CAGGGCCAGAACCAGCTCTATAACGAGCTCAATCTAGGACGAAGA
GAGGAGTACGATGTTTTGGACAAAAGACGTGGCCGGGACCCTGAG
ATGGGGGGAAAGCCGAGAAGGAAGAACCCTCAGGAAGGCCTGTAC
AATGAACTGCAGAAAGATAAGATGGCGGAGGCCTACAGTGAGATT
GGGATGAAAGGCGAGCGCCGGAGGGGCAAGGGGCACGATGGCCTT
TACCAGGGTCTCAGTACAGCCACCAAGGACACCTACGACGCCCTT
CACATGCAGGCCCTGCCCCCTCGCGGA

An example CD3zeta intracellular domain amino acid sequence:

(SEQ ID NO: 18)
KRVKFSRSADAPAYQQGQNQLYNELNLGRREEYDVLDKRRGRDPE
MGGKPRRKNPQEGLYNELQKDKMAEAYSEIGMKGERRRGKGHDGL
YQGLSTATKDTYDALHMQALPPRG

4-1BB intracellular domain nucleotide sequence:

(SEQ ID NO: 33)
AAACGGGGCAGAAAGAAACTCCTGTATATATTCAAACAACCATTT
ATGAGACCAGTACAAACTACTCAAGAGGAAGATGGCTGTAGCTGC
CGATTTCCAGAAGAAGAAGAAGGAGGATGTGAACTG

4-1BB intracellular domain amino acid sequence:

(SEQ ID NO: 34)
KRGRKKLLYIFKQPFMRPVQTTQEEDGCSCRFPEEEEGGCEL

DAP10 intracellular domain nucleotide sequence:

(SEQ ID NO: 35)
CTTTGCGCACGCCCACGCCGCAGCCCCGCCCAAGAAGATGGCAAA
GTCTACATCAACATGCCAGGCAGGGGC

DAP10 intracellular domain amino acid sequence:

(SEQ ID NO: 36)
LCARPRRSPAQEDGKVYINMPGRG

DAP12 intracellular domain nucleotide sequence:

(SEQ ID NO: 37)
TACTTCCTGGGCCGGCTGGTCCCTCGGGGGCGAGGGGCTGCGGAG
GCAGCGACCCGGAAACAGCGTATCACTGAGACCGAGTCGCCTTAT
CAGGAGCTCCAGGGTCAGAGGTCGGATGTCTACAGCGACCTCAAC
ACACAGAGGCCGTATTACAAA

DAP12 intracellular domain amino acid sequence:

(SEQ ID NO:38)
YFLGRLVPRGRGAAEAATRKQRITETESPYQELQGQRSDVYSDLN
TQRPYYK

NKG2D intracellular domain nucleotide sequence:

(SEQ ID NO: 39)
AGCGCGAACGAACGCTGCAAAAGCAAAGTGGTGCCGTGCCGCCAG
AAACAGTGGCGCACCAGCTTTGATAGCAAAAAACTGGATCTGAAC
TATAACCATTTTGAAAGCATGGAATGGAGCCATCGCAGCCGCCGC
GGCCGCATTTGGGGCATG

NKG2D intracellular domain amino acid sequence:

(SEQ ID NO: 40)
SANERCKSKVVPCRQKQWRTSFDSKKLDLNYNHFESMEWSHRSRR
GRIWGM

Any polynucleotide encompassed by the present disclosure may comprise SEQ ID NO: 15, 17, 33, 35, 37, or 39 or a sequence that is at least 70, 75, 80, 85, 90, 95, 96, 97, 98, 99, or more % identical to SEQ ID NO:15, 17, 33, 35, 37, or 39. Any polypeptide encompassed by the present disclosure may comprise SEQ ID NO:16, 18, 34, 36, 38, or 40 or a sequence that is at least 70, 75, 80, 85, 90, 95, 96, 97, 98, 99, or more % identical to SEQ ID NO:16, 18, 34, 36, 38, or 40.

### 4. Hinge

In some embodiments of the CARs, there is a hinge region between the one or more extracellular antigen binding domains and the transmembrane domain. In specific embodiments, the hinge is of a particular length, such as 10-20, 10-15, 11-20, 11-15, 12-20, 12-15, or 15-20 amino acids in length, for example. The hinge may be any suitable hinge and includes a hinge from IgG, or CD28, in some cases. In specific embodiments, the hinge is a small flexible polypeptide that connects CH2-CH3 and CH1 domains of IgG Fc. For example, one may utilize CH2-CH3 hinge (part or all) from various IgG subclasses (IgG1-4, either modified or not). However, in some cases the entire CH2-CH3 hinge is not utilized but instead a portion of the hinge is used (such as CH3 by itself or part of CH3 by itself). In particular embodiments, the CH2-CH3 hinge derived from IgG1 is utilized, and in some cases the entire CH2-CH3 hinge is used (all 229 amino acids), only the CH3 hinge (119 amino acids) is used, or a short hinge (12 amino acids) is used.

In specific cases, one can modify the identity or length of the spacer and/or hinge to optimize efficiency of the CAR. See, for example, Hudecek et al. (2014) and Jonnalagadda et al. (2015) In specific embodiments, the HLA-G CAR utilizes IgG4 hinge+CH3 or utilizes CD8a stalk, for example.

Thus, in specific embodiments the IgG hinge region that is utilized is typically IgG1 or IgG4, and in some cases the CAR comprises the CH2-CH3 domain of IgG Fc. The use of the IgG Fc domain can provide flexibility to the CAR, has low immunogenicity, facilitates detection of CAR expression using anti-Fc reagents, and allows removal of one or more CH2 or CH3 modules to accommodate different spacer lengths. However, in one embodiment mutations in certain spacers to avoid FcγR binding may improve CAR+ T cell engraftment and antitumor efficacy to avoid binding of soluble and cell surface Fc gamma receptors, for example, yet maintain the activity to mediate antigen-specific lysis. For example, one can employ IgG4-Fc spacers that have either been modified in the CH2 region. For example, the CH2 region may be mutated, including point mutations and/or deletions. Specific modifications have been demonstrated at two sites (L235E; N297Q) within the CH2 region and/or incorporate a CH2 deletion (Jonnalagadda et al, 2015). In specific embodiments, one may employ the IgG4 hinge-CH2-CH3 domain (229 aa in length) or only the hinge domain (12 aa in length) (Hudececk et al., 2015).

In specific embodiments, the hinge is from IgG, CD28, CD-8 alpha, 4-1BB, 0X40, CD3-zeta, T cell receptor α or β chain, a CD3 zeta chain, CD28, CD3e, CD45, CD4, CD5, CD8, CD9, CD16, CD22, CD33, CD37, CD64, CD80, CD86, CD134, CD137, ICOS, or CD154.

Examples of specific sequences of hinges that may be utilized include at least the following:

IgG Hinge nucleotide sequence:
(SEQ ID NO: 41)
GTACGGTCACTGTCTCTTCACAGGATCCCGCCGAGCCCAAATCTC
CTGACAAAACTCACACATGCCCACCGTGCCCAGCACCTGAACTCC
TGGGGGGACCGTCAGTCTTCCTCTTCCCCCCAAAACCCAAGGACA
CCCTCATGATCTCCCGGACCCCTGAGGTCACATGCGTGGTGGTGG
ACGTGAGCCACGAAGACCCTGAGGTCAAGTTCAACTGGTACGTGG
ACGGCGTGGAGGTGCATAATGCCAAGACAAAGCCGCGGGAGGAGC
AGTACAACAGCACGTACCGTGTGGTCAGCGTCCTCACCGTCCTGC
ACCAGGACTGGCTGAATGGCAAGGAGTACAAGTGCAAGGTCTCCA
ACAAAGCCCTCCCAGCCCCCATCGAGAAAACCATCTCCAAAGCCA
AAGGGCAGCCCCGAGAACCACAGGTGTACACCCTGCCCCCATCCC
GGGATGAGCTGACCAAGAACCAGGTCAGCCTGACCTGCCTGGTCA
AAGGCTTCTATCCCAGCGACATCGCCGTGGAGTGGGAGAGCAATG
GGCAACCGGAGAACAACTACAAGACCACGCCTCCCGTGCTGGACT
CCGACGGCTCCTTCTTCCTCTACAGCAAGCTCACCGTGGACAAGA
GCAGGTGGCAGCAGGGGAACGTCTTCTCATGCTCCGTGATGCATG
AGGCTCTGCACAACCACTACACGCAGAAGAGCCTCTCCCTGTCTC
CGGGTAAAAAAGATCCCAAATT
IgG Hinge amino acid sequence:
(SEQ ID NO:42)
TVTVSSQDPAEPKSPDKTHTCPPCPAPELLGGPSVFLFPPKPKDT
LMISRTPEVTCVVVDVSHEDPEVKFNWYVDGVEVHNAKTKPREEQ
YNSTYRVVSVLTVLHQDWLNGKEYKCKVSNKALPAPIEKTISKAK
GQPREPQVYTLPPSRDELTKNQVSLTCLVKGFYPSDIAVEWESNG
QPENNYKTTPPVLDSDGSFFLYSKLTVDKSRWQQGNVFSCSVMHE
AL
CD28 Hinge nucleotide sequence:
(SEQ ID NO: 43)
ATTGAAGTTATGTATCCTCCTCCTTACCTAGACAATGAGAAGAGC
AATGGAACCATTATCCATGTGAAAGGGAAACACCTTTGTCCAAGT
CCCCTATTTCCCGGACCTTCTAAGCCC
CD28 Hinge amino acid sequence:
(SEQ ID NO: 44)
IEVMYPPPYLDNEKSNGTIIHVKGKHLCPSPLFPGPSKP

Any polynucleotide encompassed by the present disclosure may comprise SEQ ID NO:41 or 43 or a sequence that is at least 70, 75, 80, 85, 90, 95, 96, 97, 98, 99, or more % identical to SEQ ID NO:41 or 43. Any polypeptide encompassed by the present disclosure may comprise SEQ ID NO:42 or 44 or a sequence that is at least 70, 75, 80, 85, 90, 95, 96, 97, 98, 99, or more % identical to SEQ ID NO:42 or 44.

### 5. Other Proteins

In some embodiments, one or more other proteins are utilized with an anti-HLA-G CAR of the disclosure. The one or more other proteins may be utilized for any reason, including to facilitate efficacy of the CAR itself and/or of any kind of cells expressing the CAR. In some cases, the other protein facilitates treatment of an individual receiving cells expressing the CAR as therapy, whether or not the other protein(s) directly or indirectly impact activity of the CAR or the cells. In some cases, the other protein is a suicide gene, one or more cytokines, or both. In specific embodiments, one or more other proteins are produced from a vector and ultimately are produced as two separate polypeptides. For example, the anti-HLA-G CAR and the other protein(s) may be separated by a 2A sequence or by an IRES, for example.

In specific embodiments, a cytokine such as IL-15 is utilized in conjunction with the anti-HLA-G CAR.

One example of an IL-15 nucleotide sequence is as follows:

IL-15 nucleotide sequence:
(SEQ ID NO: 21)
GCATTAGCAAGCCCCACCTGCGGAGCATCAGCATCCAGTGCTACC
TGTGCCTGCTGCTGAACAGCCACTTCCTGACCGAGGCCGGCATCC
ACGTGTTCATCCTGGGCTGCTTCAGCGCCGGACTGCCCAAGACCG
AGGCCAACTGGGTGAACGTGATCAGCGACCTGAAGAAGATCGAGG
ACCTGATCCAGAGCATGCACATCGACGCCACCCTGTACACCGAGA
GCGACGTGCACCCCAGCTGCAAGGTGACCGCCATGAAGTGCTTTC
TGCTGGAACTGCAGGTGATCAGCCTGGAAAGCGGCGACGCCAGCA
TCCACGACACCGTGGAGAACCTGATCATCCTGGCCAACAACAGCC
TGAGCAGCAACGGCAACGTGACCGAGAGCGGCTGCAAAGAGTGCG
AGGAACTGGAAGAGAAGAACATCAAAGAGTTTCTGCAGAGCTTCG
TGCACATCGTGCAGATGTTCATCAACACCAGCTGACAATT
IL-15 amino acid sequence:
(SEQ ID NO: 22)
ISKPHLRSISIQCYLCLLLNSHFLTEAGIHVFILGCFSAGLPKTE
ANWVNVISDLKKIEDLIQSMHIDATLYTESDVHPSCKVTAMKCFL
LELQVISLESGDASIHDTVENLIILANNSLSSNGNVTESGCKECE
ELEEKNIKEFLQSFVHIVQMFINTS

In cases where the CAR and another protein in the same vector are intended to be produced into two different polypeptides, a specific 2A sequence may be utilized.

In one example, an E2A nucleotide sequence is utilized as follows:

(SEQ ID NO: 19)
CAGTGTACTAATTATGCTCTCTTGAAATTGGCTGGAGATGTTGAG
AGCAATCCCGGGCCC

E2A amino acid sequence may be utilized as follows:

| (SEQ ID NO: 20) | |
|---|---|
| | QCTNYALLKLAGDVESNPGP |

Other 2A examples may be utilized and are as follows:

| | T2A: |
|---|---|
| (SEQ ID NO: 45) | |
| | EGRGSLLTCGDVEENPGP |
| | P2A: |
| (SEQ ID NO: 46) | |
| | ATNFSLLKQAGDVEENPGP |
| | F2A: |
| (SEQ ID NO: 47) | |
| | VKQTLNFDLLKLAGDVESNPGP |

The disclosure also encompasses specific CAR molecules, including for expression in any type of immune effector cells.

In one example, an anti-HLA-G CAR comprising a CD8 signal peptide, a KIR2DL4 extracellular domain, a CD28 transmembrane domain, and a CD3 zeta intracellular domain is utilized. In a vector, the CAR may be expressed with IL-15, such as may be separated from the CAR by a 2A sequence. In a specific example, such a CAR and IL-15 construct may have the following nucleotide sequence:

CD8SP-KIR2DL4EC_CD28 TMD-CD3zeta_IL15
(SEQ ID NO: 1)
ATGGCCCTGCCTGTGACAGCTCTGCTCCTCCCTCTGGCCCTGCTGCTCCA
TGCCGCCAGACCCTGGGCACACGTGGGTGGTCAGGACAAGCCCTTCTGCT
CTGCCTGGCCCAGCGCTGTGGTGCCTCAAGGAGGACACGTGACTCTTCGG
TGTCACTATCGTCGTGGGTTCAACATCTTCACGCTGTACAAGAAAGATGG
GGTCCCTGTCCCTGAGCTCTACAACAGAATATTCTGGAACAGTTTCCTCA
TTAGCCCTGTGACCCCAGCACACGCAGGGACCTACAGATGTCGAGGTTTT
CACCCGCACTCCCCCACTGAGTGGTCGGCACCCAGCAACCCCCTGGTGAT
CATGGTCACAGGTCTATATGAGAAACCTTCGCTTACAGCCCGGCTGGGCC
CCACGGTTCGCGCAGGAGAGAACGTGACCTTGTCCTGCAGCTCCCAGAGC
TCCTTTGACATCTACCATCTATCCAGGGAGGGGGAAGCCCATGAACTTAG
GCTCCCTGCAGTGCCCAGCATCAATGGAACATTCCAGGCCGACTTCCCTC
TGGGTCCTGCCACCCACGGAGAGACCTACAGATGCTTCGGCTCTTTCCAT
GGATCTCCCTACGAGTGGTCAGACCCGAGTGACCCACTGCCTGTTTCTGT
CACAGGAAACCCTTCTAGTAGTTGGCCTTCACCCACTGAACCAAGCTTCA
AAACTGGTATCGCCAGACACCTGCATTTTTGGGTGCTGGTGGTGGTTGGT
GGAGTCCTGGCTTGCTATAGCTTGCTAGTAACAGTGGCCTTTATTATTTT
CTGGGTGACGCGTAAGAAGTTCAGCAGGAGCGCAGACGCCCCCGCGTACC
AGCAGGGCCAGAACCAGCTCTATAACGAGCTCAATCTAGGACGAAGAGAG
GAGTACGATGTTTTGGACAAAAGACGTGGCCGGGACCCTGAGATGGGGGG
AAAGCCGAGAAGGAAGAACCCTCAGGAAGGCCTGTACAATGAACTGCAGA
AAGATAAGATGGCGGAGGCCTACAGTGAGATTGGGATGAAAGGCGAGCGC
CGGAGGGGCAAGGGGCACGATGGCCTTTACCAGGGTCTCAGTACAGCCAC
CAAGGACACCTACGACGCCCTTCACATGCAGGCCCTGCCCCCTCGCGGAC
CGCAGTGTACTAATTATGCTCTCTTGAAATTGGCTGGAGATGTTGAGAGC
AATCCCGGGCCCATGCGCATTAGCAAGCCCCACCTGCGGAGCATCAGCAT
CCAGTGCTACCTGTGCCTGCTGCTGAACAGCCACTTCCTGACCGAGGCCG
GCATCCACGTGTTCATCCTGGGCTGCTTCAGCGCCGGACTGCCCAAGACC
GAGGCCAACTGGGTGAACGTGATCAGCGACCTGAAGAAGATCGAGGACCT
GATCCAGAGCATGCACATCGACGCCACCCTGTACACCGAGAGCGACGTGC
ACCCCAGCTGCAAGGTGACCGCCATGAAGTGCTTTCTGCTGGAACTGCAG
GTGATCAGCCTGGAAAGCGGCGACGCCAGCATCCACGACACCGTGGAGAA
CCTGATCATCCTGGCCAACAACAGCCTGAGCAGCAACGGCAACGTGACCG
AGAGCGGCTGCAAAGAGTGCGAGGAACTGGAAGAGAAGAACATCAAAGAG
TTTCTGCAGAGCTTCGTGCACATCGTGCAGATGTTCATCAACACCAGC

A corresponding amino acid sequence for CD8SP-KIR2DLAEC_CD28 TMD-CD3zeta_IL15 is as follows:

(SEQ ID NO: 2)
MALPVTALLLPLALLLHAARPWAHVGGQDKPFCSAWPSAVVPQGGHVTLR
CHYRRGFNIFTLYKKDGVPVPELYNRIFWNSFLISPVTPAHAGTYRCRGF
HPHSPTEWSAPSNPLVIMVTGLYEKPSLTARLGPTVRAGENVTLSCSSQS
SFDIYHLSREGEAHELRLPAVPSINGTFQADFPLGPATHGETYRCFGSFH
GSPYEWSDPSDPLPVSVTGNPSSSWPSPTEPSFKTGIARHLHFWVLVVVG
GVLACYSLLVTVAFIIFWVTRKKFSRSADAPAYQQGQNQLYNELNLGRRE
EYDVLDKRRGRDPEMGGKPRRKNPQEGLYNELQKDKMAEAYSEIGMKGER
RRGKGHDGLYQGLSTATKDTYDALHMQALPPRGPQCTNYALLKLAGDVES
NPGPMRISKPHLRSISIQCYLCLLLNSHFLTEAGIHVFILGCFSAGLPKT
EANWVNVISDLKKIEDLIQSMHIDATLYTESDVHPSCKVTAMKCFLLELQ
VISLESGDASIHDTVENLIILANNSLSSNGNVTESGCKECEELEEKNIKE
FLQSFVHIVQMFINTS

In one example, an anti-HLA-G CAR comprising a CD8 signal peptide, a codon optimized KIR2DL4 extracellular domain, a CD8 transmembrane domain, a CD3 zeta intracellular domain, and a CD28 costimulatory domain is utilized. In a vector, the CAR may be expressed with IL-15, such as may be separated from the CAR by a 2A sequence. In a specific example, such a CAR and IL-15 construct may have the following nucleotide sequence:

CD8SPcoKIR2L4_EC_CD8tmd28Z15
(SEQ ID NO: 3)
ATGGGGATGGCATTGCCTGTTACAGCTCTGCTGCTGCCTCTGGCTCTGCT
TCTGCATGCTGCTAGACCTTGGGCTCACGTTGGCGGCCAGGATAAGCCTT
TTTGTTCTGCCTGGCCTAGCGCCGTGGTTCCTCAAGGTGGACACGTGACC
CTGCGGTGTCACTACAGACGGGGCTTCAACATCTTCACCCTGTACAAGAA
AGACGGCGTGCCCGTGCCTGAGCTGTACAACAGAATCTTCTGGAACAGCT
TCCTGATCAGCCCCGTGACACCAGCTCACGCCGGCACATACAGATGCAGA
GGCTTTCACCCTCACAGCCCCACAGAGTGGTCCGCTCCATCTAACCCTCT
GGTCATCATGGTCACCGGCCTGTACGAGAAGCCTAGCCTGACAGCTAGAC
TGGGCCCTACAGTTAGAGCCGGCGAGAATGTGACCCTGTCCTGTAGCAGC
CAGAGCAGCTTCGACATCTACCACCTGTCTAGAGAGGGCGAAGCCCACGA
ACTGAGACTGCCTGCCGTGCCTAGCATCAATGGCACCTTCCAGGCCGATT
TTCCACTGGGACCTGCCACACACGGCGAGACTTACAGATGCTTTGGCAGC
TTCCACGGCAGCCCTTACGAGTGGTCTGATCCTAGCGATCCTCTGCCTGT
GTCCGTGACAGGCAATCCTAGCAGCAGCTGGCCTTCTCCAACCGAGCCTA
GCTTTAAGACCGGAATCGCCCGGCATCTGCACACCACAACACCAGCACCT
AGACCTCCAACTCCAGCTCCTACAATCGCCAGCCAGCCTCTGTCTCTGAG
GCCTGAAGCTTGTAGACCTGCTGCTGGCGGAGCCGTGCATACCAGAGGAC
TGGATTTCGCCTGCGATATCTACATCTGGGCCCCTCTGGCTGGAACATGT
GGCGTGCTGCTGCTGAGCCTCGTGATCACAAAACGCGTGAAGTTCAGCAG
GAGCGCAGACGCCCCCGCGTACCAGCAGGGCCAGAACCAGCTCTATAACG
AGCTCAATCTAGGACGAAGAGAGGAGTACGATGTTTTGGACAAAAGACGT
GGCCGGGACCCTGAGATGGGGGGAAAGCCGAGAAGGAAGAACCCTCAGGA
AGGCCTGTACAATGAACTGCAGAAAGATAAGATGGCGGAGGCCTACAGTG
AGATTGGGATGAAAGGCGAGCGCCGGAGGGGCAAGGGGCACGATGGCCTT
TACCAGGGTCTCAGTACAGCCACCAAGGACACCTACGACGCCCTTCACAT
GCAGGCCCTGCCCCCTCGCGGACCGCAGTGTACTAATTATGCTCTCTTGA
AATTGGCTGGAGATGTTGAGAGCAATCCCGGGCCCATGCGCATTAGCAAG
CCCCACCTGCGGAGCATCAGCATCCAGTGCTACCTGTGCCTGCTGCTGAA
CAGCCACTTCCTGACCGAGGCCGGCATCCACGTGTTCATCCTGGGCTGCT
TCAGCGCCGGACTGCCCAAGACCGAGGCCAACTGGGTGAACGTGATCAGC
GACCTGAAGAAGATCGAGGACCTGATCCAGAGCATGCACATCGACGCCAC
CCTGTACACCGAGAGCGACGTGCACCCCAGCTGCAAGGTGACCGCCATGA
AGTGCTTTCTGCTGGAACTGCAGGTGATCAGCCTGGAAAGCGGCGACGCC
AGCATCCACGACACCGTGGAGAACCTGATCATCCTGGCCAACAACAGCCT
GAGCAGCAACGGCAACGTGACCGAGAGCGGCTGCAAAGAGTGCGAGGAAC
TGGAAGAGAAGAACATCAAAGAGTTTCTGCAGAGCTTCGTGCACATCGTG
CAGATGTTCATCAACACCAGCTGA

A corresponding amino acid sequence for CD8SPcoKIR2L4_EC_CD8tmd28Z15 is as follows:

(SEQ ID NO: 4)
MGMALPVTALLLPLALLLHAARPWAHVGGQDKPFCSAWPSAVVPQGGHVT
LRCHYRRGFNIFTLYKKDGVPVPELYNRIFWNSFLISPVTPAHAGTYRCR
GFHPHSPTEWSAPSNPLVIMVTGLYEKPSLTARLGPTVRAGENVTLSCSS
QSSFDIYHLSREGEAHELRLPAVPSINGTFQADFPLGPATHGETYRCFGS
FHGSPYEWSDPSDPLPVSVTGNPSSSWPSPTEPSFKTGIARHLHTTTPAP
RPPTPAPTIASQPLSLRPEACRPAAGGAVHTRGLDFACDIYIWAPLAGTC
GVLLLSLVITKRVKFSRSADAPAYQQGQNQLYNELNLGRREEYDVLDKRR
GRDPEMGGKPRRKNPQEGLYNELQKDKMAEAYSEIGMKGERRRGKGHDGL
YQGLSTATKDTYDALHMQALPPRGPQCTNYALLKLAGDVESNPGPMRISK
PHLRSISIQCYLCLLLNSHFLTEAGIHVFILGCFSAGLPKTEANWVNVIS
DLKKIEDLIQSMHIDATLYTESDVHPSCKVTAMKCFLLELQVISLESGDA
SIHDTVENLIILANNSLSSNGNVTESGCKECEELEEKNIKEFLQSFVHIV
QMFINTS

In some embodiments, a particular CAR utilizing an anti-HLA-G antibody is employed. In a specific case, a construct encoding the CAR comprises inducible caspase 9, a CAR comprising MEM-G/11 antibody (with the variable heavy chain N-terminal to the variable light chain), a CD28 hinge, a CD28 costimulatory domain, and CD3zeta, wherein the expression construct also expresses IL-15. The separate polypeptides may be separated by 2A sequences. Exemplary sequences for such a construct are as follows:

iC9MEMVHVL28HingeCD28CD3ZIL15
(SEQ ID NO: 74)
ATGCTCGAGGGAGTGCAGGTGGAAACCATCTCCCCAGGCGACGGGCGCAC
CTTCCCCAAGCGCGGCCAGACCTGCGTGGTGCACTACACCGGGATGCTTG
AAGATGGAAAGAAAGTTGATTCCTCCCGGGACAGAAACAAGCCCTTTAAG
TTTATGCTAGGCAAGCAGGAGGTGATCCGAGGCTGGGAAGAAGGGGTTGC
CCAGATGAGTGTGGGTCAGAGAGCCAAACTGACTATATCTCCAGATTATG
CCTATGGTGCCACTGGGCACCCAGGCATCATCCCACCACATGCCACTCTC
GTCTTCGATGTGGAGCTTCTAAAACTGGAATCTGGCGGTGGATCCGGAGT
CGACGGATTTGGTGATGTCGGTGCTCTTGAGAGTTTGAGGGGAAATGCAG
ATTTGGCTTACATCCTGAGCATGGAGCCCTGTGGCCACTGCCTCATTATC
AACAATGTGAACTTCTGCCGTGAGTCCGGGCTCCGCACCCGCACTGGCTC
CAACATCGACTGTGAGAAGTTGCGGCGTCGCTTCTCCTCGCTGCATTTCA
TGGTGGAGGTGAAGGGCGACCTGACTGCCAAGAAAATGGTGCTGGCTTTG
CTGGAGCTGGCGCAGCAGGACCACGGTGCTCTGGACTGCTGCGTGGTGGT
CATTCTCTCTCACGGCTGTCAGGCCAGCCACCTGCAGTTCCCAGGGGCTG
TCTACGGCACAGATGGATGCCCTGTGTCGGTCGAGAAGATTGTGAACATC
TTCAATGGGACCAGCTGCCCCAGCCTGGGAGGGAAGCCCAAGCTCTTTTT
CATCCAGGCCTGTGGTGGGGAGCAGAAAGATCATGGGTTTGAGGTGGCCT
CCACTTCCCCTGAAGACGAGTCCCCTGGCAGTAACCCCGAGCCAGATGCC
ACCCCGTTCCAGGAAGGTTTGAGGACCTTCGACCAGCTGGACGCCATATC
TAGTTTGCCCACACCCAGTGACATCTTTGTGTCCTACTCTACTTTCCCAG
GTTTTGTTTCCTGGAGGGACCCCAAGAGTGGCTCCTGGTACGTTGAGACC
CTGGACGACATCTTTGAGCAGTGGGCTCACTCTGAAGACCTGCAGTCCCT
CCTGCTTAGGGTCGCTAATGCTGTTTCGGTGAAAGGGATTTATAAACAGA
TGCCTGGTTGCTTTAATTTCCTCCGGAAAAAACTTTTCTTTAAAACATCA
GCTTCGCGAGCCGAGGGCAGGGGAAGTCTTCTAACATGCGGGGACGTGGA
GGAAAATCCCGGGCCCATGGAGTTTGGGCTGAGCTGGCTTTTTCTTGTGG
CTATTTTAAAAGGTGTCCAGTGCTCTAGAGAGaaagtgcagctggtggaa
agcggcggcggcctggtgaaaccgggcggcagcctgaaactgagctgcag
cgcgagcggctttccgtttagcgattattatatgtattgggtgcgccaga
ccccggaaaaacgcctggaatgggtggcgaccattagcgatgatgatgat
tatacctattatccggatagcatgaaaggccgctttaccattagccgcga
taacgcgaaaaacaacctgtatctgcagatgagcagcctgaaaagcgaag
ataccgcgatgtattattgcagccgcggcatttattatggcagcagcccg
tttgcgtattggggccagggcaccctggtgaccgtgagcgcgTTGGAAAT
AAAGGGCTCTACAAGCGGCTCAGGAAAACCTGGATCAGGCGAAGGGTCTA
CGgatattgtgctgacccagagcccggcgagcctggatgtgagcctgggc
cagcgcgcgaccattagctgccgcgcgagcaaaagcgtgagcaccagcgg
ctatagctatatgcattggtatcagcagaaaccgggccagagcccgaaac
tgctgatttatctggcgagcaaccgcgaaagcggcgtgccggcgcgcttt
agcggcagcggcagcggcaccgattttaccctgaacattcatccggtgga
agaagaagatgcggcgacctattattgccagcatagccgcgaatttccga
cctttggcgcgggcaccaaactggaactgaaaCaCGTACGccCCATTGAA
GTTATGTATCCTCCTCCTTACCTAGACAATGAGAAGAGCAATGGAACCAT
TATCCATGTGAAAGGGAAACACCTTTGTCCAAGTCCCCTATTTCCCGGAC
CTTCTAAGCCCAAATTTTGGGTGCTGGTGGTGGTTGGTGGAGTCCTGGCT
TGCTATAGCTTGCTAGTAACAGTGGCCTTTATTATTTTCTGGGTGAGGAG
TAAGAGGAGCAGGCTCCTGCACAGTGACTACATGAACATGACTCCCCGCC
GCCCCGGGCCCACCCGCAAGCATTACCAGCCCTATGCCCCACCACGCGAC
TTCGCAGCCTATCGCTCACGCGTGAAGTTCAGCAGGAGCGCAGACGCCCC
CGCGTACCAGCAGGGCCAGAACCAGCTCTATAACGAGCTCAATCTAGGAC
GAAGAGAGGAGTACGATGTTTTGGACAAAAGACGTGGCCGGGACCCTGAG
ATGGGGGGAAAGCCGAGAAGGAAGAACCCTCAGGAAGGCCTGTACAATGA
ACTGCAGAAAGATAAGATGGCGGAGGCCTACAGTGAGATTGGGATGAAAG
GCGAGCGCCGGAGGGGCAAGGGGCACGATGGCCTTTACCAGGGTCTCAGT
ACAGCCACCAAGGACACCTACGACGCCCTTCACATGCAGGCCCTGCCCCC
TCGCGGACCGCAGTGTACTAATTATGCTCTCTTGAAATTGGCTGGAGATG
TTGAGAGCAATCCCGGGCCCATGCGCATTAGCAAGCCCCACCTGCGGAGC
ATCAGCATCCAGTGCTACCTGTGCCTGCTGCTGAACAGCCACTTCCTGAC
CGAGGCCGGCATCCACGTGTTCATCCTGGGCTGCTTCAGCGCCGGACTGC
CCAAGACCGAGGCCAACTGGGTGAACGTGATCAGCGACCTGAAGAAGATC
GAGGACCTGATCCAGAGCATGCACATCGACGCCACCCTGTACACCGAGAG
CGACGTGCACCCCAGCTGCAAGGTGACCGCCATGAAGTGCTTTCTGCTGG
AACTGCAGGTGATCAGCCTGGAAAGCGGCGACGCCAGCATCCACGACACC
GTGGAGAACCTGATCATCCTGGCCAACAACAGCCTGAGCAGCAACGGCAA
CGTGACCGAGAGCGGCTGCAAAGAGTGCGAGGAACTGGAAGAGAAGAACA
TCAAAGAGTTTCTGCAGAGCTTCGTGCACATCGTGCAGATGTTCATCAAC
ACCAGCTGA

An example of a polypeptide for iC9MEMVHVL28HingeCD28CD3ZIL15 may comprise:

(SEQ ID NO: 75)
MLEGVQVETISPGDGRTFPKRGQTCVVHYTGMLEDGKKVDSSRDRNKPFK
FMLGKQEVIRGWEEGVAQMSVGQRAKLTISPDYAYGATGHPGIIPPHATL
VFDVELLKLESGGGSGVDGFGDVGALESLRGNADLAYILSMEPCGHCLII
NNVNFCRESGLRTRTGSNIDCEKLRRRFSSLHFMVEVKGDLTAKKMVLAL
LELAQQDHGALDCCVVVILSHGCQASHLQFPGAVYGTDGCPVSVEKIVNI
FNGTSCPSLGGKPKLFFIQACGGEQKDHGFEVASTSPEDESPGSNPEPDA
TPFQEGLRTFDQLDAISSLPTPSDIFVSYSTFPGFVSWRDPKSGSWYVET
LDDIFEQWAHSEDLQSLLLRVANAVSVKGIYKQMPGCFNFLRKKLFFKTS
ASRAEGRGSLLTCGDVEENPGPMEFGLSWLFLVAILKGVQCSREKVQLVE
SGGGLVKPGGSLKLSCSASGFPFSDYYMYWVRQTPEKRLEWVATISDDDD
YTYYPDSMKGRFTISRDNAKNNLYLQMSSLKSEDTAMYYCSRGIYYGSSP
FAYWGQGTLVTVSALEIKGSTSGSGKPGSGEGSTDIVLTQSPASLDVSLG
QRATISCRASKSVSTSGYSYMHWYQQKPGQSPKLLIYLASNRESGVPARF
SGSGSGTDFTLNIHPVEEEDAATYYCQHSREFPTFGAGTKLELKHVRPIE
VMYPPPYLDNEKSNGTIIHVKGKHLCPSPLFPGPSKPKFWVLVVVGGVLA
CYSLLVTVAFIIFWVRSKRSRLLHSDYMNMTPRRPGPTRKHYQPYAPPRD
FAAYRSRVKFSRSADAPAYQQGQNQLYNELNLGRREEYDVLDKRRGRDPE
MGGKPRRKNPQEGLYNELQKDKMAEAYSEIGMKGERRRGKGHDGLYQGLS
TATKDTYDALHMQALPPRGPQCTNYALLKLAGDVESNPGPMRISKPHLRS
ISIQCYLCLLLNSHFLTEAGIHVFILGCFSAGLPKTEANWVNVISDLKKI
EDLIQSMHIDATLYTESDVHPSCKVTAMKCFLLELQVISLESGDASIHDT
VENLIILANNSLSSNGNVTESGCKECEELEEKNIKEFLQSFVHIVQMFIN
TS*

In a specific case, a construct encoding the CAR comprises inducible caspase 9, a CAR comprising MEM-G/11antibody (with the variable heavy chain N-terminal to the variable light chain), a CD28 hinge, a DAP10 costimulatory domain, and CD3zeta, wherein the expression construct also expresses IL-15. The separate polypeptides may be separated by 2A sequences. Exemplary sequences for such a construct are as follows:

iC9MEMVHVL28HingeDAP10CD3ZIL15
(SEQ ID NO: 76)
ATGCTCGAGGGAGTGCAGGTGGAAACCATCTCCCCAGGCGACGGGCGCAC
CTTCCCCAAGCGCGGCCAGACCTGCGTGGTGCACTACACCGGGATGCTTG
AAGATGGAAAGAAAGTTGATTCCTCCCGGGACAGAAACAAGCCCTTTAAG
TTTATGCTAGGCAAGCAGGAGGTGATCCGAGGCTGGGAAGAAGGGGTTGC
CCAGATGAGTGTGGGTCAGAGAGCCAAACTGACTATATCTCCAGATTATG
CCTATGGTGCCACTGGGCACCCAGGCATCATCCCACCACATGCCACTCTC
GTCTTCGATGTGGAGCTTCTAAAACTGGAATCTGGCGGTGGATCCGGAGT
CGACGGATTTGGTGATGTCGGTGCTCTTGAGAGTTTGAGGGGAAATGCAG
ATTTGGCTTACATCCTGAGCATGGAGCCCTGTGGCCACTGCCTCATTATC
AACAATGTGAACTTCTGCCGTGAGTCCGGGCTCCGCACCCGCACTGGCTC
CAACATCGACTGTGAGAAGTTGCGGCGTCGCTTCTCCTCGCTGCATTTCA
TGGTGGAGGTGAAGGGCGACCTGACTGCCAAGAAAATGGTGCTGGCTTTG
CTGGAGCTGGCGCAGCAGGACCACGGTGCTCTGGACTGCTGCGTGGTGGT
CATTCTCTCTCACGGCTGTCAGGCCAGCCACCTGCAGTTCCCAGGGGCTG
TCTACGGCACAGATGGATGCCCTGTGTCGGTCGAGAAGATTGTGAACATC
TTCAATGGGACCAGCTGCCCCAGCCTGGGAGGGAAGCCCAAGCTCTTTTT
CATCCAGGCCTGTGGTGGGGAGCAGAAAGATCATGGGTTTGAGGTGGCCT
CCACTTCCCCTGAAGACGAGTCCCCTGGCAGTAACCCCGAGCCAGATGCC
ACCCCGTTCCAGGAAGGTTTGAGGACCTTCGACCAGCTGGACGCCATATC
TAGTTTGCCCACACCCAGTGACATCTTTGTGTCCTACTCTACTTTCCCAG
GTTTTGTTTCCTGGAGGGACCCCAAGAGTGGCTCCTGGTACGTTGAGACC
CTGGACGACATCTTTGAGCAGTGGGCTCACTCTGAAGACCTGCAGTCCCT
CCTGCTTAGGGTCGCTAATGCTGTTTCGGTGAAAGGGATTTATAAACAGA
TGCCTGGTTGCTTTAATTTCCTCCGGAAAAAACTTTTCTTTAAAACATCA
GCTTCGCGAGCCGAGGGCAGGGGAAGTCTTCTAACATGCGGGGACGTGGA
GGAAAATCCCGGGCCCATGGAGTTTGGGCTGAGCTGGCTTTTTCTTGTGG
CTATTTTAAAAGGTGTCCAGTGCTCTAGAGAGaaagtgcagctggtggaa
agcggcggcggcctggtgaaaccgggcggcagcctgaaactgagctgcag
cgcgagcggctttccgtttagcgattattatatgtattgggtgcgccaga
ccccggaaaaacgcctggaatgggtggcgaccattagcgatgatgatgat
tatacctattatccggatagcatgaaaggccgctttaccattagccgcga
taacgcgaaaaacaacctgtatctgcagatgagcagcctgaaaagcgaag
ataccgcgatgtattattgcagccgcggcatttattatggcagcagcccg
tttgcgtattggggccagggcaccctggtgaccgtgagcgcgTTGGAAAT
AAAGGGCTCTACAAGCGGCTCAGGAAAACCTGGATCAGGCGAAGGGTCTA
CGgatattgtgctgacccagagcccggcgagcctggatgtgagcctgggc
cagcgcgcgaccattagctgccgcgcgagcaaaagcgtgagcaccagcgg
ctatagctatatgcattggtatcagcagaaaccgggccagagcccgaaac
tgctgatttatctggcgagcaaccgcgaaagcggcgtgccggcgcgcttt
agcggcagcggcagcggcaccgattttaccctgaacattcatccggtgga
agaagaagatgcggcgacctattattgccagcatagccgcgaatttccga
cctttggcgcgggcaccaaactggaactgaaaCCGTACGCCATTGAAGTT
ATGTATCCTCCTCCTTACCTAGACAATGAGAAGAGCAATGGAACCATTAT
CCATGTGAAAGGGAAACACCTTTGTCCAAGTCCCCTATTTCCCGGACCTT
CTAAGCCCAAATTTTGGGTGCTGGTGGTGGTTGGTGGAGTCCTGGCTTGC
TATAGCTTGCTAGTAACAGTGGCCTTTATTATTTTCTGGGTGCTTTGCGC
ACGCCCACGCCGCAGCCCCGCCCAAGAAGATGGCAAAGTCTACATCAACA
TGCCAGGCAGGGGCCGCGTGAAGTTCAGCAGGAGCGCAGACGCCCCCGCG
TACCAGCAGGGCCAGAACCAGCTCTATAACGAGCTCAATCTAGGACGAAG
AGAGGAGTACGATGTTTTGGACAAAAGACGTGGCCGGGACCCTGAGATGG
GGGGAAAGCCGAGAAGGAAGAACCCTCAGGAAGGCCTGTACAATGAACTG
CAGAAAGATAAGATGGCGGAGGCCTACAGTGAGATTGGGATGAAAGGCGA
GCGCCGGAGGGGCAAGGGGCACGATGGCCTTTACCAGGGTCTCAGTACAG
CCACCAAGGACACCTACGACGCCCTTCACATGCAGGCCCTGCCCCCTCGC
GGACCGCAGTGTACTAATTATGCTCTCTTGAAATTGGCTGGAGATGTTGA
GAGCAATCCCGGGCCCATGCGCATTAGCAAGCCCCACCTGCGGAGCATCA
GCATCCAGTGCTACCTGTGCCTGCTGCTGAACAGCCACTTCCTGACCGAG
GCCGGCATCCACGTGTTCATCCTGGGCTGCTTCAGCGCCGGACTGCCCAA
GACCGAGGCCAACTGGGTGAACGTGATCAGCGACCTGAAGAAGATCGAGG
ACCTGATCCAGAGCATGCACATCGACGCCACCCTGTACACCGAGAGCGAC
GTGCACCCCAGCTGCAAGGTGACCGCCATGAAGTGCTTTCTGCTGGAACT
GCAGGTGATCAGCCTGGAAAGCGGCGACGCCAGCATCCACGACACCGTGG
AGAACCTGATCATCCTGGCCAACAACAGCCTGAGCAGCAACGGCAACGTG
ACCGAGAGCGGCTGCAAAGAGTGCGAGGAACTGGAAGAGAAGAACATCAA
AGAGTTTCTGCAGAGCTTCGTGCACATCGTGCAGATGTTCATCAACACCA
GCTGA.

An example of a polypeptide encoded by

iC9MEMVHVL28HingeDAP10CD3ZIL15 may comprise:
(SEQ ID NO: 77)
MLEGVQVETISPGDGRTFPKRGQTCVVHYTGMLEDGKKVDSSRDRNKPFK
FMLGKQEVIRGWEEGVAQMSVGQRAKLTISPDYAYGATGHPGIIPPHATL
VFDVELLKLESGGGSGVDGFGDVGALESLRGNADLAYILSMEPCGHCLII
NNVNFCRESGLRTRTGSNIDCEKLRRRFSSLHFMVEVKGDLTAKKMVLAL
LELAQQDHGALDCCVVVILSHGCQASHLQFPGAVYGTDGCPVSVEKIVNI
FNGTSCPSLGGKPKLFFIQACGGEQKDHGFEVASTSPEDESPGSNPEPDA
TPFQEGLRTFDQLDAISSLPTPSDIFVSYSTFPGFVSWRDPKSGSWYVET
LDDIFEQWAHSEDLQSLLLRVANAVSVKGIYKQMPGCFNFLRKKLFFKTS
ASRAEGRGSLLTCGDVEENPGPMEFGLSWLFLVAILKGVQCSREKVQLVE
SGGGLVKPGGSLKLSCSASGFPFSDYYMYWVRQTPEKRLEWVATISDDDD
YTYYPDSMKGRFTISRDNAKNNLYLQMSSLKSEDTAMYYCSRGIYYGSSP
FAYWGQGTLVTVSALEIKGSTSGSGKPGSGEGSTDIVLTQSPASLDVSLG
QRATISCRASKSVSTSGYSYMHWYQQKPGQSPKLLIYLASNRESGVPARF
SGSGSGTDFTLNIHPVEEEDAATYYCQHSREFPTFGAGTKLELKPYAIEV
MYPPPYLDNEKSNGTIIHVKGKHLCPSPLFPGPSKPKFWVLVVVGGVLAC
YSLLVTVAFIIFWVLCARPRRSPAQEDGKVYINMPGRGRVKFSRSADAPA
YQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR
GPQCTNYALLKLAGDVESNPGPMRISKPHLRSISIQCYLCLLLNSHFLTE
AGIHVFILGCFSAGLPKTEANWVNVISDLKKIEDLIQSMHIDATLYTESD
VHPSCKVTAMKCFLLELQVISLESGDASIHDTVENLIILANNSLSSNGNV
TESGCKECEELEEKNIKEFLQSFVHIVQMFINTS*

In a specific case, a construct encoding the CAR comprises inducible caspase 9, a CAR comprising 87G antibody (with the variable heavy chain N-terminal to the variable light chain), a CD28 hinge, a CD28 costimulatory domain, and CD3zeta, wherein the expression construct also expresses IL-15. The separate polypeptides may be separated by 2A sequences. Exemplary sequences for such a construct are as follows:

iC9-87GVHVL28HingeCD28CD3ZIL15
(SEQ ID NO: 78)
ATGCTCGAGGGAGTGCAGGTGGAAACCATCTCCCCAGGCGACGGGCGCAC
CTTCCCCAAGCGCGGCCAGACCTGCGTGGTGCACTACACCGGGATGCTTG
AAGATGGAAAGAAAGTTGATTCCTCCCGGGACAGAAACAAGCCCTTTAAG
TTTATGCTAGGCAAGCAGGAGGTGATCCGAGGCTGGGAAGAAGGGGTTGC
CCAGATGAGTGTGGGTCAGAGAGCCAAACTGACTATATCTCCAGATTATG
CCTATGGTGCCACTGGGCACCCAGGCATCATCCCACCACATGCCACTCTC
GTCTTCGATGTGGAGCTTCTAAAACTGGAATCTGGCGGTGGATCCGGAGT
CGACGGATTTGGTGATGTCGGTGCTCTTGAGAGTTTGAGGGGAAATGCAG
ATTTGGCTTACATCCTGAGCATGGAGCCCTGTGGCCACTGCCTCATTATC
AACAATGTGAACTTCTGCCGTGAGTCCGGGCTCCGCACCCGCACTGGCTC
CAACATCGACTGTGAGAAGTTGCGGCGTCGCTTCTCCTCGCTGCATTTCA
TGGTGGAGGTGAAGGGCGACCTGACTGCCAAGAAAATGGTGCTGGCTTTG
CTGGAGCTGGCGCAGCAGGACCACGGTGCTCTGGACTGCTGCGTGGTGGT
CATTCTCTCTCACGGCTGTCAGGCCAGCCACCTGCAGTTCCCAGGGGCTG
TCTACGGCACAGATGGATGCCCTGTGTCGGTCGAGAAGATTGTGAACATC
TTCAATGGGACCAGCTGCCCCAGCCTGGGAGGGAAGCCCAAGCTCTTTTT
CATCCAGGCCTGTGGTGGGGAGCAGAAAGATCATGGGTTTGAGGTGGCCT
CCACTTCCCCTGAAGACGAGTCCCCTGGCAGTAACCCCGAGCCAGATGCC
ACCCCGTTCCAGGAAGGTTTGAGGACCTTCGACCAGCTGGACGCCATATC
TAGTTTGCCCACACCCAGTGACATCTTTGTGTCCTACTCTACTTTCCCAG
GTTTTGTTTCCTGGAGGGACCCCAAGAGTGGCTCCTGGTACGTTGAGACC
CTGGACGACATCTTTGAGCAGTGGGCTCACTCTGAAGACCTGCAGTCCCT
CCTGCTTAGGGTCGCTAATGCTGTTTCGGTGAAAGGGATTTATAAACAGA
TGCCTGGTTGCTTTAATTTCCTCCGGAAAAAACTTTTCTTTAAAACATCA
GCTTCGCGAGCCGAGGGCAGGGGAAGTCTTCTAACATGCGGGGACGTGGA
GGAAAATCCCGGGCCCATGGAGTTTGGGCTGAGCTGGCTTTTTCTTGTGG
CTATTTTAAAAGGTGTCCAGTGCTCTAGAGAGgaagtgaaactggtggaa
agcggcggcagcctggtgcagccgggcggcagcctgaaactgagctgcgc
ggcgagcggctttagctttagcagctataccatgagctgggtgcgccaga
ccccgaaaaaacgcctggaatgggggcgtatgtgagcaacggcgcgggca
ccacctattatccggatagcctgaaaggccgctttaccattagccgcgat
aacgcgaaaaacaccctgcatctgctgatgaccagcctgaaaagcgaaga
taccgcgatttattattgcgcgcgccattattatggcagctatcattttg
attattggggccagggcaccaccctgattgtgagcagcTTGGAAATAAAG
GGCTCTACAAGCGGCTCAGGAAAACCTGGATCAGGCGAAGGGTCTACGga
aaccaccgtgacccagagcccggcgagcctgagcgtggcgaccggcgaaa
aagtgaccattcgctgcattaccagcaccgatattgatgatgatatgaac
tggtatcagcagaaaccgggcgaaccgccgaaactgctgattagcgaaga
taacattctgcgcccgggcgtgccgagccgctttagcagcagcggctatg
gcaccgattttgtgtttaccattgaaaacaccctgagcgaagatgtggcg
gattattattgcctgcagagcgataacatgccgctgacctttggcggcgg
cacccgcctggaaattaaaCaCGTACGccCCATTGAAGTTATGTATCCTC
CTCCTTACCTAGACAATGAGAAGAGCAATGGAACCATTATCCATGTGAAA
GGGAAACACCTTTGTCCAAGTCCCCTATTTCCCGGACCTTCTAAGCCCAA
ATTTTGGGTGCTGGTGGTGGTTGGTGGAGTCCTGGCTTGCTATAGCTTGC
TAGTAACAGTGGCCTTTATTATTTTCTGGGTGAGGAGTAAGAGGAGCAGG
CTCCTGCACAGTGACTACATGAACATGACTCCCCGCCGCCCCGGGCCCAC
CCGCAAGCATTACCAGCCCTATGCCCCACCACGCGACTTCGCAGCCTATC
GCTCACGCGTGAAGTTCAGCAGGAGCGCAGACGCCCCCGCGTACCAGCAG
GGCCAGAACCAGCTCTATAACGAGCTCAATCTAGGACGAAGAGAGGAGTA
CGATGTTTTGGACAAAAGACGTGGCCGGGACCCTGAGATGGGGGGAAAGC
CGAGAAGGAAGAACCCTCAGGAAGGCCTGTACAATGAACTGCAGAAAGAT
AAGATGGCGGAGGCCTACAGTGAGATTGGGATGAAAGGCGAGCGCCGGAG
GGGCAAGGGGCACGATGGCCTTTACCAGGGTCTCAGTACAGCCACCAAGG
ACACCTACGACGCCCTTCACATGCAGGCCCTGCCCCCTCGCGGACCGCAG
TGTACTAATTATGCTCTCTTGAAATTGGCTGGAGATGTTGAGAGCAATCC
CGGGCCCATGCGCATTAGCAAGCCCCACCTGCGGAGCATCAGCATCCAGT
GCTACCTGTGCCTGCTGCTGAACAGCCACTTCCTGACCGAGGCCGGCATC
CACGTGTTCATCCTGGGCTGCTTCAGCGCCGGACTGCCCAAGACCGAGGC
CAACTGGGTGAACGTGATCAGCGACCTGAAGAAGATCGAGGACCTGATCC
AGAGCATGCACATCGACGCCACCCTGTACACCGAGAGCGACGTGCACCCC
AGCTGCAAGGTGACCGCCATGAAGTGCTTTCTGCTGGAACTGCAGGTGAT
CAGCCTGGAAAGCGGCGACGCCAGCATCCACGACACCGTGGAGAACCTGA
TCATCCTGGCCAACAACAGCCTGAGCAGCAACGGCAACGTGACCGAGAGC
GGCTGCAAAGAGTGCGAGGAACTGGAAGAGAAGAACATCAAAGAGTTTCT
GCAGAGCTTCGTGCACATCGTGCAGATGTTCATCAACACCAGCTGA

An example of a polypeptide encoded by iC9-87GVHVL28HingeCD28CD3ZIL15 may comprise:

(SEQ ID NO: 79)
MLEGVQVETISPGDGRTFPKRGQTCVVHYTGMLEDGKKVDSSRDRNKPFK
FMLGKQEVIRGWEEGVAQMSVGQRAKLTISPDYAYGATGHPGIIPPHATL
VFDVELLKLESGGGSGVDGFGDVGALESLRGNADLAYILSMEPCGHCLII
NNVNFCRESGLRTRTGSNIDCEKLRRRFSSLHFMVEVKGDLTAKKMVLAL
LELAQQDHGALDCCVVVILSHGCQASHLQFPGAVYGTDGCPVSVEKIVNI
FNGTSCPSLGGKPKLFFIQACGGEQKDHGFEVASTSPEDESPGSNPEPDA
TPFQEGLRTFDQLDAISSLPTPSDIFVSYSTFPGFVSWRDPKSGSWYVET
LDDIFEQWAHSEDLQSLLLRVANAVSVKGIYKQMPGCFNFLRKKLFFKTS
ASRAEGRGSLLTCGDVEENPGPMEFGLSWLFLVAILKGVQCSREEVKLVE
SGGSLVQPGGSLKLSCAASGFSFSSYTMSWVRQTPKKRLEWVAYVSNGAG
TTYYPDSLKGRFTISRDNAKNTLHLLMTSLKSEDTAIYYCARHYYGSYHF
DYWGQGTTLIVSSLEIKGSTSGSGKPGSGEGSTETTVTQSPASLSVATGE
KVTIRCITSTDIDDDMNWYQQKPGEPPKLLISEDNILRPGVPSRFSSSGY
GTDFVFTIENTLSEDVADYYCLQSDNMPLTFGGGTRLEIKHVRPIEVMYP
PPYLDNEKSNGTIIHVKGKHLCPSPLFPGPSKPKFWVLVVVGGVLACYSL
LVTVAFIIFWVRSKRSRLLHSDYMNMTPRRPGPTRKHYQPYAPPRDFAAY
RSRVKFSRSADAPAYQQGQNQLYNELNLGRREEYDVLDKRRGRDPEMGGK
PRRKNPQEGLYNELQKDKMAEAYSEIGMKGERRRGKGHDGLYQGLSTATK
DTYDALHMQALPPRGPQCTNYALLKLAGDVESNPGPMRISKPHLRSISIQ
CYLCLLLNSHFLTEAGIHVFILGCFSAGLPKTEANWVNVISDLKKIEDLI
QSMHIDATLYTESDVHPSCKVTAMKCFLLELQVISLESGDASIHDTVENL
IILANNSLSSNGNVTESGCKECEELEEKNIKEFLQSFVHIVQMFINTS*

In a specific case, a construct encoding the CAR comprises inducible caspase 9, a CAR comprising 87G antibody (with the variable heavy chain N-terminal to the variable light chain), a CD28 hinge, a DAP10 costimulatory domain, and CD3zeta, wherein the expression construct also expresses IL-15. The separate polypeptides may be separated by 2A sequences. Exemplary sequences for such a construct are as follows:

iC9-87GVHVL28HingeDAP10CD3ZIL15
(SEQ ID NO: 80)
ATGCTCGAGGGAGTGCAGGTGGAAACCATCTCCCCAGGCGACGGGCGCAC
CTTCCCCAAGCGCGGCCAGACCTGCGTGGTGCACTACACCGGGATGCTTG
AAGATGGAAAGAAAGTTGATTCCTCCCGGGACAGAAACAAGCCCTTTAAG
TTTATGCTAGGCAAGCAGGAGGTGATCCGAGGCTGGGAAGAAGGGGTTGC
CCAGATGAGTGTGGGTCAGAGAGCCAAACTGACTATATCTCCAGATTATG
CCTATGGTGCCACTGGGCACCCAGGCATCATCCCACCACATGCCACTCTC
GTCTTCGATGTGGAGCTTCTAAAACTGGAATCTGGCGGTGGATCCGGAGT
CGACGGATTTGGTGATGTCGGTGCTCTTGAGAGTTTGAGGGGAAATGCAG
ATTTGGCTTACATCCTGAGCATGGAGCCCTGTGGCCACTGCCTCATTATC
AACAATGTGAACTTCTGCCGTGAGTCCGGGCTCCGCACCCGCACTGGCTC
CAACATCGACTGTGAGAAGTTGCGGCGTCGCTTCTCCTCGCTGCATTTCA
TGGTGGAGGTGAAGGGCGACCTGACTGCCAAGAAAATGGTGCTGGCTTTG
CTGGAGCTGGCGCAGCAGGACCACGGTGCTCTGGACTGCTGCGTGGTGGT
CATTCTCTCTCACGGCTGTCAGGCCAGCCACCTGCAGTTCCCAGGGGCTG
TCTACGGCACAGATGGATGCCCTGTGTCGGTCGAGAAGATTGTGAACATC
TTCAATGGGACCAGCTGCCCCAGCCTGGGAGGGAAGCCCAAGCTCTTTTT
CATCCAGGCCTGTGGTGGGGAGCAGAAAGATCATGGGTTTGAGGTGGCCT
CCACTTCCCCTGAAGACGAGTCCCCTGGCAGTAACCCCGAGCCAGATGCC
ACCCCGTTCCAGGAAGGTTTGAGGACCTTCGACCAGCTGGACGCCATATC
TAGTTTGCCCACACCCAGTGACATCTTTGTGTCCTACTCTACTTTCCCAG
GTTTTGTTTCCTGGAGGGACCCCAAGAGTGGCTCCTGGTACGTTGAGACC
CTGGACGACATCTTTGAGCAGTGGGCTCACTCTGAAGACCTGCAGTCCCT
CCTGCTTAGGGTCGCTAATGCTGTTTCGGTGAAAGGGATTTATAAACAGA
TGCCTGGTTGCTTTAATTTCCTCCGGAAAAAACTTTTCTTTAAAACATCA
GCTTCGCGAGCCGAGGGCAGGGGAAGTCTTCTAACATGCGGGGACGTGGA
GGAAAATCCCGGGCCCATGGAGTTTGGGCTGAGCTGGCTTTTTCTTGTGG
CTATTTTAAAAGGTGTCCAGTGCTCTAGAGAGgaagtgaaactggtggaa
agcggcggcagcctggtgcagccgggcggcagcctgaaactgagctgcgc
ggcgagcggctttagctttagcagctataccatgagctgggtgcgccaga
ccccgaaaaaacgcctggaatgggtggcgtatgtgagcaacggcgcgggc
accacctattatccggatagcctgaaaggccgctttaccattagccgcga
taacgcgaaaaacaccctgcatctgctgatgaccagcctgaaaagcgaag
ataccgcgatttattattgcgcgcgccattattatggcagctatcatttt
gattattggggccagggcaccaccctgattgtgagcagcTTGGAAATAAA
GGGCTCTACAAGCGGCTCAGGAAAACCTGGATCAGGCGAAGGGTCTACGg
aaaccaccgtgacccagagcccggcgagcctgagcgtggcgaccggcgaa
aaagtgaccattcgctgcattaccagcaccgatattgatgatgatatgaa
ctggtatcagcagaaaccgggcgaaccgccgaaactgctgattagcgaag
ataacattctgcgcccgggcgtgccgagccgctttagcagcagcggctat
ggcaccgattttgtgtttaccattgaaaacaccctgagcgaagatgtggc
ggattattattgcctgcagagcgataacatgccgctgacctttggcggcg
gcacccgcctggaaattaaaCCGTACGCCATTGAAGTTATGTATCCTCCT
CCTTACCTAGACAATGAGAAGAGCAATGGAACCATTATCCATGTGAAAGG
GAAACACCTTTGTCCAAGTCCCCTATTTCCCGGACCTTCTAAGCCCAAAT
TTTGGGTGCTGGTGGTGGTTGGTGGAGTCCTGGCTTGCTATAGCTTGCTA
GTAACAGTGGCCTTTATTATTTTCTGGGTGCTTTGCGCACGCCCACGCCG
CAGCCCCGCCCAAGAAGATGGCAAAGTCTACATCAACATGCCAGGCAGGG
GCCGCGTGAAGTTCAGCAGGAGCGCAGACGCCCCCGCGTACCAGCAGGGC
CAGAACCAGCTCTATAACGAGCTCAATCTAGGACGAAGAGAGGAGTACGA
TGTTTTGGACAAAAGACGTGGCCGGGACCCTGAGATGGGGGGAAAGCCGA
GAAGGAAGAACCCTCAGGAAGGCCTGTACAATGAACTGCAGAAAGATAAG
ATGGCGGAGGCCTACAGTGAGATTGGGATGAAAGGCGAGCGCCGGAGGGG
CAAGGGGCACGATGGCCTTTACCAGGGTCTCAGTACAGCCACCAAGGACA
CCTACGACGCCCTTCACATGCAGGCCCTGCCCCCTCGCGGACCGCAGTGT
ACTAATTATGCTCTCTTGAAATTGGCTGGAGATGTTGAGAGCAATCCCGG
GCCCATGCGCATTAGCAAGCCCCACCTGCGGAGCATCAGCATCCAGTGCT
ACCTGTGCCTGCTGCTGAACAGCCACTTCCTGACCGAGGCCGGCATCCAC
GTGTTCATCCTGGGCTGCTTCAGCGCCGGACTGCCCAAGACCGAGGCCAA
CTGGGTGAACGTGATCAGCGACCTGAAGAAGATCGAGGACCTGATCCAGA
GCATGCACATCGACGCCACCCTGTACACCGAGAGCGACGTGCACCCCAGC
TGCAAGGTGACCGCCATGAAGTGCTTTCTGCTGGAACTGCAGGTGATCAG
CCTGGAAAGCGGCGACGCCAGCATCCACGACACCGTGGAGAACCTGATCA
TCCTGGCCAACAACAGCCTGAGCAGCAACGGCAACGTGACCGAGAGCGGC
TGCAAAGAGTGCGAGGAACTGGAAGAGAAGAACATCAAAGAGTTTCTGCA
GAGCTTCGTGCACATCGTGCAGATGTTCATCAACACCAGCTGA.

An example of a polypeptide encoded by iC9-87GVHVL28HingeDAP10CD3ZIL15 may comprise

(SEQ ID NO: 81)
MLEGVQVETISPGDGRTFPKRGQTCVVHYTGMLEDGKKVDSSRDRNKPFK
FMLGKQEVIRGWEEGVAQMSVGQRAKLTISPDYAYGATGHPGIIPPHATL
VFDVELLKLESGGGSGVDGFGDVGALESLRGNADLAYILSMEPCGHCLII
NNVNFCRESGLRTRTGSNIDCEKLRRRFSSLHFMVEVKGDLTAKKMVLAL
LELAQQDHGALDCCVVVILSHGCQASHLQFPGAVYGTDGCPVSVEKIVNI
FNGTSCPSLGGKPKLFFIQACGGEQKDHGFEVASTSPEDESPGSNPEPDA
TPFQEGLRTFDQLDAISSLPTPSDIFVSYSTFPGFVSWRDPKSGSWYVET
LDDIFEQWAHSEDLQSLLLRVANAVSVKGIYKQMPGCFNFLRKKLFFKTS
ASRAEGRGSLLTCGDVEENPGPMEFGLSWLFLVAILKGVQCSREEVKLVE
SGGSLVQPGGSLKLSCAASGFSFSSYTMSWVRQTPKKRLEWVAYVSNGAG
TTYYPDSLKGRFTISRDNAKNTLHLLMTSLKSEDTAIYYCARHYYGSYHF
DYWGQGTTLIVSSLEIKGSTSGSGKPGSGEGSTETTVTQSPASLSVATGE
KVTIRCITSTDIDDDMNWYQQKPGEPPKLLISEDNILRPGVPSRFSSSGY
GTDFVFTIENTLSEDVADYYCLQSDNMPLTFGGGTRLEIKPYAIEVMYPP
PYLDNEKSNGTIIHVKGKHLCPSPLFPGPSKPKFWVLVVVGGVLACYSLL
VTVAFIIFWVLCARPRRSPAQEDGKVYINMPGRGRVKFSRSADAPAYQQG
QNQLYNELNLGRREEYDVLDKRRGRDPEMGGKPRRKNPQEGLYNELQKDK
MAEAYSEIGMKGERRRGKGHDGLYQGLSTATKDTYDALHMQALPPRGPQC
TNYALLKLAGDVESNPGPMRISKPHLRSISIQCYLCLLLNSHFLTEAGIH
VFILGCFSAGLPKTEANWVNVISDLKKIEDLIQSMHIDATLYTESDVHPS
CKVTAMKCFLLELQVISLESGDASIHDTVENLIILANNSLSSNGNVTESG
CKECEELEEKNIKEFLQSFVHIVQMFINTS*.

### C. T Cell Receptor (TCR)

In some embodiments, a HLA-G-targeting genetically engineered antigen receptor includes recombinant TCRs and/or TCRs cloned from naturally occurring T cells, or one or more portions thereof. A “T cell receptor” or “TCR” refers to a molecule that contains a variable α and β chains (also known as TCRα and TCRβ, respectively) or a variable γ and δ chains (also known as TCRγ and TCRδ, respectively) and that is capable of specifically binding to an antigen peptide bound to a MHC receptor. In some embodiments, the TCR is in the αβ form.

Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., 1990; Chothia et al., 1988; Lefranc et al., 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains (e.g., α-chain, β-chain) can contain two immunoglobulin domains, a variable domain (e.g., Vₐ or Vp; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., α-chain constant domain or Cₐ, typically amino acids 117 to 259 based on Kabat, β-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3εchains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD35 chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds. In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, a high-affinity T cell clone can be isolated from a patient, and the TCR isolated. In some embodiments, the T cells can be a cultured T cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al., 2009 and Cohen et al., 2005). In some embodiments, phage display is used to isolate TCRs against a target antigen (see, e.g., Varela-Rohena et al., 2008 and Li, 2005). In some embodiments, the TCR or antigen-binding portion thereof can be synthetically generated from knowledge of the sequence of the TCR.

### III. Cytokines

One or more cytokines may be utilized with one or more HLA-G-targeting genetically engineered receptors, such as HLA-G-specific CARs. In some cases, one or more cytokines are present on the same vector molecule as the engineered receptor, although in other cases they are on separate vector molecules. In particular embodiments, one or more cytokines are co-expressed from the same vector as the engineered receptor. One or more cytokines may be produced as a separate polypeptide from the HLA-G-specific receptor. As one example, Interleukin-15 (IL-15), is utilized. IL-15 may be employed because, for example, it is tissue restricted and only under pathologic conditions is it observed at any level in the serum, or systemically. IL-15 possesses several attributes that are desirable for adoptive therapy. IL-15 is a homeostatic cytokine that induces development and cell proliferation of natural killer cells, promotes the eradication of established tumors via alleviating functional suppression of tumor-resident cells, and inhibits activation-induced cell death. In addition to IL-15, other cytokines are envisioned. These include, but are not limited to, cytokines, chemokines, and other molecules that contribute to the activation and proliferation of cells used for human application. As one example, the one or more cytokines are IL-15, IL-12, IL-2, IL-18, IL-21, IL-23, IL-7, or combination thereof. NK cells expressing IL-15 may be utilized and are capable of continued supportive cytokine signaling, which is useful for their survival post-infusion.

In specific embodiments, NK cells express one or more exogenously provided cytokines. The cytokine may be exogenously provided to the NK cells because it is expressed from an expression vector within the cell and/or because it is provided in a culture medium of the cells. In an alternative case, an endogenous cytokine in the cell is upregulated upon manipulation of regulation of expression of the endogenous cytokine, such as genetic recombination at the promoter site(s) of the cytokine. In cases wherein the cytokine is provided on an expression construct to the cell, the cytokine may be encoded from the same vector as a suicide gene. The cytokine may be expressed as a separate polypeptide molecule from a suicide gene and as a separate polypeptide from an engineered receptor of the cell. In some embodiments, the present disclosure concerns co-utilization of CAR and/or TCR vectors with IL-15, particularly in NK cells.

### IV. Suicide Genes

In particular embodiments, a suicide gene is utilized in conjunction with cell therapy of any kind to control its use and allow for termination of the cell therapy at a desired event and/or time. The suicide gene is employed in transduced cells for the purpose of eliciting death for the transduced cells when needed. The HLA-G-targeting cells of the present disclosure that have been modified to harbor a vector encompassed by the disclosure may comprise one or more suicide genes. In some embodiments, the term “suicide gene” as used herein is defined as a gene which, upon administration of a prodrug or other agent, effects transition of a gene product to a compound which kills its host cell. In other embodiments, a suicide gene encodes a gene product that is, when desired, targeted by an agent (such as an antibody) that targets the suicide gene product.

Examples of suicide gene/prodrug combinations which may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside. The E. coli purine nucleoside phosphorylase, a so-called suicide gene that converts the prodrug 6-methylpurine deoxyriboside to toxic purine 6-methylpurine, may be used. Other examples of suicide genes used with prodrug therapy are the E. coli cytosine deaminase gene and the HSV thymidine kinase gene.

Exemplary suicide genes also include CD20, CD52, EGFRv3, or inducible caspase 9. In one embodiment, a truncated version of EGFR variant III (EGFRv3) may be used as a suicide antigen that can be ablated by Cetuximab. Further suicide genes known in the art that may be used in the present disclosure include Purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), and Thymidine phosphorylase (TP).

In particular embodiments, vectors that encode the HLA-G-targeting CAR, or any vector in a NK cell encompassed herein, include one or more suicide genes. The suicide gene may or may not be on the same vector as a HLA-G-targeting CAR. In cases wherein the suicide gene is present on the same vector as the HLA-G-targeting CAR, the suicide gene and the CAR may be separated by an IRES or 2A element, for example.

### V. Vectors

The HLA-G-targeting CARs may be delivered to the recipient immune cells by any suitable vector, including by a viral vector or by a non-viral vector. Examples of viral vectors include at least retroviral, lentiviral, adenoviral, or adeno-associated viral vectors. Examples of non-viral vectors include at least plasmids, transposons, lipids, nanoparticles, and so forth.

In cases wherein the immune cell is transduced with a vector encoding the HLA-G-targeting receptor and also requires transduction of another gene or genes into the cell, such as a suicide gene and/or cytokine and/or an optional therapeutic gene product, the HLA-G-targeting receptor, suicide gene, cytokine, and optional therapeutic gene may or may not be comprised on or with the same vector. In some cases, the HLA-G-targeting CAR, suicide gene, cytokine, and optional therapeutic gene are expressed from the same vector molecule, such as the same viral vector molecule. In such cases, the expression of the HLA-G-targeting CAR, suicide gene, cytokine, and optional therapeutic gene may or may not be regulated by the same regulatory element(s). When the HLA-G-targeting CAR, suicide gene, cytokine, and optional therapeutic gene are on the same vector, they may or may not be expressed as separate polypeptides. In cases wherein they are expressed as separate polypeptides, they may be separated on the vector by a 2A element or IRES element (or both kinds may be used on the same vector once or more than once), for example.

### A. General Embodiments

One of skill in the art would be well-equipped to construct a vector through standard recombinant techniques (see, for example, Sambrook et al., 2001 and Ausubel et al., 1996, both incorporated herein by reference) for the expression of the antigen receptors of the present disclosure.

### 1. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells may be comprised of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters, for example. In cases wherein the vector is utilized for the generation of cancer therapy, a promoter may be effective under conditions of hypoxia.

### 2. Promoter/Enhancers

The expression constructs provided herein comprise a promoter to drive expression of the antigen receptor and other cistron gene products. A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, for example, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp-) promoter systems. In addition to producing nucleic acid sequences of promoters and enhancers synthetically, sequences may be produced using recombinant cloning and/or nucleic acid amplification technology, including PCR™, in connection with the compositions disclosed herein. Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e. g., beta actin promoter, GADPH promoter, metallothionein promoter; and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at GenBank®, accession no. X05244, nucleotide 283-341) or a mouse mammary tumor promoter (available from the ATCC, Cat. No. ATCC 45007). In certain embodiments, the promoter is CMV IE, dectin-1, dectin-2, human CD11c, F4/80, SM22, RSV, SV40, Ad MLP, beta-actin, MHC class I or MHC class II promoter, however any other promoter that is useful to drive expression of the therapeutic gene is applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

### 3. Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

As detailed elsewhere herein, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the equine rhinitis A virus (E2A) or the F2A (Foot-and-mouth disease virus 2A) or a “2A-like” sequence (e.g., Thosea asigna virus 2A; T2A) or porcine teschovirus-1 (P2A). In specific embodiments, in a single vector the multiple 2A sequences are non-identical, although in alternative embodiments the same vector utilizes two or more of the same 2A sequences. Examples of 2A sequences are provided in US 2011/0065779 which is incorporated by reference herein in its entirety.

### 4. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively a replication origin of other extra-chromosomally replicating virus as described above or an autonomously replicating sequence (ARS) can be employed.

### 5. Selection and Screenable Markers

In some embodiments, NK cells comprising a HLA-G-targeting receptor construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

### B. Multicistronic Vectors

In particular embodiments, the HLA-G-targeting receptor, optional suicide gene, optional cytokine, and/or optional therapeutic gene are expressed from a multicistronic vector (The term “cistron” as used herein refers to a nucleic acid sequence from which a gene product may be produced). In specific embodiments, the multicistronic vector encodes the HLA-G-targeting receptor, the suicide gene, and at least one cytokine, and/or engineered receptor, such as a T-cell receptor and/or an additional non-HLA-G-targeting CAR. In some cases, the multicistronic vector encodes at least one HLA-G-targeting CAR, at least one TNF-alpha mutant, and at least one cytokine. The cytokine may be of a particular type of cytokine, such as human or mouse or any species. In specific cases, the cytokine is IL15, IL12, IL2, IL18, and/or IL21.

In certain embodiments, the present disclosure provides a flexible, modular system (the term “modular” as used herein refers to a cistron or component of a cistron that allows for interchangeability thereof, such as by removal and replacement of an entire cistron or of a component of a cistron, respectively, for example by using standard recombination techniques) utilizing a polycistronic vector having the ability to express multiple cistrons at substantially identical levels. The system may be used for cell engineering allowing for combinatorial expression (including overexpression) of multiple genes. In specific embodiments, one or more of the genes expressed by the vector includes one, two, or more antigen receptors. The multiple genes may comprise, but are not limited to, CARs, TCRs, cytokines, chemokines, homing receptors, CRISPR/Cas9-mediated gene mutations, decoy receptors, cytokine receptors, chimeric cytokine receptors, and so forth. The vector may further comprise: (1) one or more reporters, for example fluorescent or enzymatic reporters, such as for cellular assays and animal imaging; (2) one or more cytokines or other signaling molecules; and/or (3) a suicide gene.

In specific cases, the vector may comprise at least 4 cistrons separated by cleavage sites of any kind, such as 2A cleavage sites. The vector may or may not be Moloney Murine Leukemia Virus (MoMLV or MMLV)-based including the 3′ and 5′ LTR with the psi packaging sequence in a pUC19 backbone. The vector may comprise 4 or more cistrons with three or more 2A cleavage sites and multiple ORFs for gene swapping. The system allows for combinatorial overexpression of multiple genes (7 or more) that are flanked by restriction site(s) for rapid integration through subcloning, and the system also includes at least three 2A self-cleavage sites, in some embodiments. Thus, the system allows for expression of multiple CARs, TCRs, signaling molecules, cytokines, cytokine receptors, and/or homing receptors. This system may also be applied to other viral and non-viral vectors, including but not limited lentivirus, adenovirus AAV, as well as non-viral plasmids.

The modular nature of the system also enables efficient subcloning of a gene into each of the 4 cistrons in the polycistronic expression vector and the swapping of genes, such as for rapid testing. Restriction sites strategically located in the polycistronic expression vector allow for swapping of genes with efficiency.

Embodiments of the disclosure encompass systems that utilize a polycistronic vector wherein at least part of the vector is modular, for example by allowing removal and replacement of one or more cistrons (or component(s) of one or more cistrons), such as by utilizing one or more restriction enzyme sites whose identity and location are specifically selected to facilitate the modular use of the vector. The vector also has embodiments wherein multiple of the cistrons are translated into a single polypeptide and processed into separate polypeptides, thereby imparting an advantage for the vector to express separate gene products in substantially equimolar concentrations.

The vector of the disclosure is configured for modularity to be able to change one or more cistrons of the vector and/or to change one or more components of one or more particular cistrons. The vector may be designed to utilize unique restriction enzyme sites flanking the ends of one or more cistrons and/or flanking the ends of one or more components of a particular cistron.

Embodiments of the disclosure include polycistronic vectors comprising at least two, at least three, or at least four cistrons each flanked by one or more restriction enzyme sites, wherein at least one cistron encodes for at least one antigen receptor. In some cases, two, three, four, or more of the cistrons are translated into a single polypeptide and cleaved into separate polypeptides, whereas in other cases multiple of the cistrons are translated into a single polypeptide and cleaved into separate polypeptides. Adjacent cistrons on the vector may be separated by a self cleavage site, such as a 2A self cleavage site. In some cases each of the cistrons express separate polypeptides from the vector. On particular cases, adjacent cistrons on the vector are separated by an IRES element.

In certain embodiments, the present disclosure provides a system for cell engineering allowing for combinatorial expression, including overexpression, of multiple cistrons that may include one, two, or more antigen receptors, for example. In particular embodiments, the use of a polycistronic vector as described herein allows for the vector to produce equimolar levels of multiple gene products from the same mRNA. The multiple genes may comprise, but are not limited to, CARs, TCRs, cytokines, chemokines, homing receptors, CRISPR/Cas9-mediated gene mutations, decoy receptors, cytokine receptors, chimeric cytokine receptors, and so forth. The vector may further comprise one or more fluorescent or enzymatic reporters, such as for cellular assays and animal imaging. The vector may also comprise a suicide gene product for termination of cells harboring the vector when they are no longer needed or become deleterious to a host to which they have been provided.

In specific embodiments, the vector is a viral vector (retroviral vector, lentiviral vector, adenoviral vector, or adeno-associated viral vector, for example) or a non-viral vector. The vector may comprise a Moloney Murine Leukemia Virus (MMLV) 5′ LTR, 3′ LTR, and/or psi packaging element. In specific cases, the psi packaging is incorporated between the 5′ LTR and the antigen receptor coding sequence. The vector may or may not comprise pUC19 sequence. In some aspects of the vector, at least one cistron encodes for a cytokine (IL-15, IL-7, IL-21, IL-23, IL-18, IL-12, or IL-2, for example), chemokine, cytokine receptor, and/or homing receptor.

When 2A cleavages sites are utilized in the vector, the 2A cleavage site may comprise a P2A, T2A, E2A and/or F2A site.

A restriction enzyme site may be of any kind and may include any number of bases in its recognition site, such as between 4 and 8 bases; the number of bases in the recognition site may be at least 4, 5, 6, 7, 8, or more. The site when cut may produce a blunt cut or sticky ends. The restriction enzyme may be of Type I, Type II, Type III, or Type IV, for example. Restriction enzyme sites may be obtained from available databases, such as Integrated relational Enzyme database (IntEnz) or BRENDA (The Comprehensive Enzyme Information System).

Exemplary vectors may be circular and by convention, where position 1 (12 o'clock position at the top of the circle, with the rest of the sequence in clock-wise direction) is set at the start of 5′ LTR.

In embodiments wherein self-cleaving 2A peptides are utilized, the 2A peptides may be 18-22 amino-acid (aa)-long viral oligopeptides that mediate “cleavage” of polypeptides during translation in eukaryotic cells. The designation “2A” refers to a specific region of the viral genome and different viral 2As have generally been named after the virus they were derived from. The first discovered 2A was F2A (foot-and-mouth disease virus), after which E2A (equine rhinitis A virus), P2A (porcine teschovirus-1 2A), and T2A (thosea asigna virus 2A) were also identified. The mechanism of 2A-mediated “self-cleavage” was discovered to be ribosome skipping the formation of a glycyl-prolyl peptide bond at the C-terminus of the 2A.

In specific cases, the vector may be a γ-retroviral transfer vector. The retroviral transfer vector may comprises a backbone based on a plasmid, such as the pUC19 plasmid (large fragment (2.63 kb) in between HindIII and EcoRI restriction enzyme sites). The backbone may carry viral components from Moloney Murine Leukemia Virus (MoMLV) including 5′ LTR, psi packaging sequence, and 3′ LTR. LTRs are long terminal repeats found on either side of a retroviral provirus, and in the case of a transfer vector, brackets the genetic cargo of interest, such as HLA-G-targeting CARs and associated components. The psi packaging sequence, which is a target site for packaging by nucleocapsid, is also incorporated in cis, sandwiched between the 5′ LTR and the CAR coding sequence. Thus, the basic structure of an example of a transfer vector can be configured as such: pUC19 sequence—5′ LTR—psi packaging sequence—genetic cargo of interest—3′ LTR—pUC19 sequence. This system may also be applied to other viral and non-viral vectors, including but not limited lentivirus, adenovirus AAV, as well as non-viral plasmids.

### VI. Cells

The present disclosure encompasses immune cells or stem cells of any kind that harbor at least one vector that encodes a HLA-G-targeting receptor and that also may encode at least one cytokine and/or at least one suicide gene. In some cases, different vectors encode the CAR vs. encodes the suicide gene and/or cytokine. The immune cells, including NK cells, may be derived from cord blood (including pooled cord blood from multiple sources), peripheral blood, induced pluripotent stem cells (iPSCs), hematopoietic stem cells (HSCs), bone marrow, or a mixture thereof. The NK cells may be derived from a cell line such as, but not limited to, NK-92 cells, for example. The NK cell may be a cord blood mononuclear cell, such as a CD56⁺ NK cell.

The present disclosure encompasses immune or other cells of any kind, including conventional T cells, gamma-delta T cells, NKT and invariant NK T cells, regulatory T cells, macrophages, B cells, dendritic cells, mesenchymal stromal cells (MSCs), or a mixture thereof.

In some cases, the cells have been expanded in the presence of an effective amount of universal antigen presenting cells (UAPCs), including in any suitable ratio. The cells may be cultured with the UAPCs at a ratio of 10:1 to 1:10; 9:1 to 1:9; 8:1 to 1:8; 7:1 to 1:7; 6:1 to 1:6; 5:1 to 1:5; 4:1 to 1:4; 3:1 to 1:3; 2:1 to 1:2; or 1:1, including at a ratio of 1:2, for example. In some cases, the NK cells were expanded in the presence of IL-2, such as at a concentration of 10-500, 10-400, 10-300, 10-200, 10-100, 10-50, 100-500, 100-400, 100-300, 100-200, 200-500, 200-400, 200-300, 300-500, 300-400, or 400-500 U/mL.

Following genetic modification with the vector(s), the NK cells may be immediately infused or may be stored. In certain aspects, following genetic modification, the cells may be propagated for days, weeks, or months ex vivo as a bulk population within about 1, 2, 3, 4, 5 days or more following gene transfer into cells. In a further aspect, the transfectants are cloned and a clone demonstrating presence of a single integrated or episomally maintained expression cassette or plasmid, and expression of the HLA-G-targeting CAR is expanded ex vivo. The clone selected for expansion demonstrates the capacity to specifically recognize and lyse HLA-G-expressing target cells. The recombinant immune cells may be expanded by stimulation with IL-2, or other cytokines that bind the common gamma-chain (e.g., IL-7, IL-12, IL-15, IL-21, IL-23, and others). The recombinant immune cells may be expanded by stimulation with artificial antigen presenting cells. In a further aspect, the genetically modified cells may be cryopreserved.

Embodiments of the disclosure encompass cells that express one or more HLA-G-targeting CARs and one or more suicide genes as encompassed herein. The NK cell comprises a recombinant nucleic acid that encodes one or more HLA-G-targeting CARs and one or more engineered nonsecretable, membrane bound TNF-alpha mutant polypeptides, in specific embodiments. In specific embodiments, in addition to expressing one or more HLA-G-targeting CARs and TNF-alpha mutant polypeptides, the cell also comprises a nucleic acid that encodes one or more therapeutic gene products.

The cells may be obtained from an individual directly or may be obtained from a depository or other storage facility. The cells as therapy may be autologous or allogeneic with respect to the individual to which the cells are provided as therapy.

The cells may be from an individual in need of therapy for a medical condition, and following their manipulation to express the HLA-G-targeting CAR, optional suicide gene, optional cytokine(s), and optional therapeutic gene product(s) (using standard techniques for transduction and expansion for adoptive cell therapy, for example), they may be provided back to the individual from which they were originally sourced. In some cases, the cells are stored for later use for the individual or another individual.

The immune cells may be comprised in a population of cells, and that population may have a majority that are transduced with one or more HLA-G-targeting receptors and/or one or more suicide genes and/or one or more cytokines. A cell population may comprise 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% of immune cells that are transduced with one or more HLA-G-targeting receptors and/or one or more suicide genes and/or one or more cytokines. The one or more HLA-G-targeting receptors and/or one or more suicide genes and/or one or more cytokines may be separate polypeptides.

The immune cells may be produced with the one or more HLA-G-targeting receptors and/or one or more suicide genes and/or one or more cytokines for the intent of being modular with respect to a specific purpose. For example, cells may be generated, including for commercial distribution, expressing a HLA-G-targeting CARs and/or one or more suicide genes and/or one or more cytokines (or distributed with a nucleic acid that encodes the mutant for subsequent transduction), and a user may modify them to express one or more other genes of interest (including therapeutic genes) dependent upon their intended purpose(s). For instance, an individual interested in treating HLA-G-positive cells, including HLA-G-positive cancer, may obtain or generate suicide gene-expressing cells (or heterologous cytokine-expressing cells) and modify them to express a receptor comprising a HLA-G-specific scFv, or vice versa.

In particular embodiments, NK cells are utilized, and the genome of the transduced NK cells expressing the one or more HLA-G-targeting CARs and/or one or more suicide genes and/or one or more cytokines may be modified. The genome may be modified in any manner, but in specific embodiments the genome is modified by CRISPR gene editing, for example. The genome of the cells may be modified to enhance effectiveness of the cells for any purpose.

### VII. Gene Editing of HLA-G-Specific CAR Cells

In particular embodiments, cells comprising at least a HLA-G-specific engineered receptor are gene edited to modify expression of one or more endogenous genes in the cell. In specific cases, the HLA-G-specific CAR cells are modified to have reduced levels of expression of one or more endogenous genes, including inhibition of expression of one or more endogenous genes (that may be referred to as knocked out). Such cells may or may not be expanded.

In particular cases, one or more endogenous genes of the HLA-G-specific CAR cells are modified, such as disrupted in expression where the expression is reduced in part or in full. In specific cases, one or more genes are knocked down or knocked out using processes of the disclosure. In specific cases, multiple genes are knocked down or knocked out, and this may or may not occur in the same step in their production. The genes that are edited in the HLA-G-specific CAR cells may be of any kind, but in specific embodiments the genes are genes whose gene products inhibit activity and/or proliferation of the HLA-G-specific CAR cells, including HLA-G-specific CAR NK cells, such as those derived from cord blood, as one example. In specific cases the genes that are edited in the HLA-G-specific CAR cells allow the HLA-G-specific CAR cells to work more effectively in a tumor microenvironment. In specific cases, the genes are one or more of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, CD5, CD7, and CD38. In specific embodiments, the TGFBR2, CISH, and/or CD38 genes are knocked out or knocked down in the HLA-G-specific CAR cells.

In some embodiments, the gene editing is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins; in some embodiments, CpF1 is utilized instead of Cas9. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions or alterations as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpfl (Cas12a) homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). In some cases, Cpfl (Cas12a) may be used as an endonuclease instead of Cas9. The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

### VIII. Methods of Treatment

In various embodiments, diseased or other cells expressing endogenous HLA-G on their surface are targeted for the purpose of improving a medical condition in an individual that has the medical condition or for the purpose of reducing the risk or delaying the severity and/or onset of the medical condition in an individual. In specific cases, cancer cells expressing endogenous HLA-G are targeted for the purpose of killing the cancer cells.

HLA-G-targeting CAR constructs, nucleic acid sequences, vectors, immune cells and so forth as contemplated herein, and/or pharmaceutical compositions comprising the same, are used for the prevention, treatment or amelioration of a cancerous disease, such as a tumorous disease. In particular embodiments, the pharmaceutical composition of the present disclosure may be particularly useful in preventing, ameliorating and/or treating cancer, including cancers that express HLA-G and that may or may not be solid tumors, for example.

The immune cells for which the HLA-G-targeting receptor is utilized may be NK cells, T cells, gamma delta T cells, alpha beta T cells, or NKT or invariant NKT (INKT), or invariant NKT cells engineered for cell therapy for mammals, in particular embodiments. In such cases where the cells are NK cells, the NK cell therapy may be of any kind and the NK cells may be of any kind. In specific embodiments, the cells are NK cells that have been engineered to express one or more HLA-G-targeting CARs and/or one or more suicide genes and/or one or more cytokines. In specific embodiments, the cells are NK cells that are transduced with a HLA-G-targeting CAR.

In particular embodiments, the present disclosure contemplates, in part, HLA-G CAR-expressing cells, HLA-G-targeting CAR constructs, HLA-G-targeting CAR nucleic acid molecules and HLA-G-targeting CAR vectors that can be administered either alone or in any combination using standard vectors and/or gene delivery systems, and in at least some aspects, together with a pharmaceutically acceptable carrier or excipient. In certain embodiments, subsequent to administration, the nucleic acid molecules or vectors may be stably integrated into the genome of the subject.

In specific embodiments, viral vectors may be used that are specific for certain cells or tissues and persist in NK cells. Suitable pharmaceutical carriers and excipients are well known in the art. The compositions prepared according to the disclosure can be used for the prevention or treatment or delaying the above identified diseases.

Furthermore, the disclosure relates to a method for the prevention, treatment or amelioration of a tumorous disease comprising the step of administering to a subject in the need thereof an effective amount of cells that express a HLA-G-targeting CAR, a nucleic acid sequence, a vector, as contemplated herein and/or produced by a process as contemplated herein.

Possible indications for administration of the composition(s) of the exemplary HLA-G-targeting CAR cells are cancerous diseases, including tumorous diseases, including B cell malignancies, multiple myeloma, breast cancer, glioblastoma, renal cancer, pancreatic cancer, or lung cancer, for example. Exemplary indications for administration of the composition(s) of HLA-G-targeting CAR cells are cancerous diseases, including any malignancies that express HLA-G. The administration of the composition(s) of the disclosure is useful for all stages (I, II, III, or IV) and types of cancer, including for minimal residual disease, early cancer, advanced cancer, and/or metastatic cancer and/or refractory cancer, for example.

The disclosure further encompasses co-administration protocols with other compounds, e.g. bispecific antibody constructs, targeted toxins or other compounds, which act via immune cells. The clinical regimen for co-administration of the inventive compound(s) may encompass co-administration at the same time, before or after the administration of the other component. Particular combination therapies include chemotherapy, radiation, surgery, hormone therapy, or other types of immunotherapy.

Embodiments relate to a kit comprising a HLA-G-targeting CAR construct as defined herein, a nucleic acid sequence as defined herein, a vector as defined herein and/or a host cell (such as an immune cell) as defined herein. It is also contemplated that the kit of this disclosure comprises a pharmaceutical composition as described herein above, either alone or in combination with further medicaments to be administered to an individual in need of medical treatment or intervention.

### A. Pharmaceutical Compositions

Also provided herein are pharmaceutical compositions and formulations comprising transduced NK cells and a pharmaceutically acceptable carrier. The transduced cells may be comprised in a media suitable for transfer to an individual and/or media suitable for preservation, such as cryopreservation, including prior to transfer to an individual.

Pharmaceutical compositions and formulations as described herein can be prepared by mixing the active ingredients (such as the cells) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

### B. Combination Therapies

In certain embodiments, the compositions and methods of the present embodiments involve an immune cell population (including NK cell population) in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, hormone therapy, oncolytic viruses, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

In particular embodiments, in addition to the inventive cell therapy of the disclosure, the individual may have been provided, may be provided, and/or may will be provided a specific additional therapy for cancer, including one or more of surgery, radiation, immunotherapy (other than the cell therapy of the present disclosure), hormone therapy, gene therapy, chemotherapy, and so forth.

An immune cell therapy may be administered before, during, after, or in various combinations relative to an additional cancer therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an immune cell therapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or cell therapy of the present embodiments to a patient will follow general protocols for the administration of such compounds, taking into account the toxicity, if any, of the agents. Therefore, in some embodiments there is a step of monitoring toxicity that is attributable to combination therapy.

### 1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclophosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegal1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

### 2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### 3. Immunotherapy

The skilled artisan will understand that additional immunotherapies than CARs may be used alone or in combination or in conjunction with methods of the embodiments. Any antibody encompassed herein may be employed in therapeutic molecules other than CARS, including in bi-specific or multi-specific antibodies. In the context of cancer treatment, immunotherapeutics, generally, rely on the use of immune effector cells or agents and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells.

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLAR (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

### 4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### 5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### IX. Proteins

As used herein, a “protein” or “polypeptide” refers to a molecule comprising at least three amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of any one of SEQ ID NOs: 1-81.

In some embodiments, the protein, polypeptide, or polynucleotide may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) contiguous amino acids or nucleotides of any of SEQ ID NOs: 1-81.

In some embodiments, the polypeptide, protein, or polynucleotide may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides, respectively, of SEQ ID NOs: 1-81 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with any one of SEQ ID NOS:1-81.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of any of SEQ ID NOS: 1-49 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides of any of SEQ ID NOS: 1-81.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

### X. Kits of the Disclosure

Any of the compositions described herein may be comprised in a kit. In a non-limiting example, cells, reagents to produce cells, vectors, and reagents to produce vectors and/or components thereof may be comprised in a kit. In certain embodiments, NK cells may be comprised in a kit, and they may or may not yet express a HLA-G-targeting receptor, an optional cytokine, or an optional suicide gene. Such a kit may or may not have one or more reagents for manipulation of cells. Such reagents include small molecules, proteins, nucleic acids, antibodies, buffers, primers, nucleotides, salts, and/or a combination thereof, for example. Nucleotides that encode one or more HLA-G-targeting CARs, suicide gene products, and/or cytokines may be included in the kit. Proteins, such as cytokines or antibodies, including monoclonal antibodies, may be included in the kit. Nucleotides that encode components of engineered CAR receptors may be included in the kit, including reagents to generate same.

In particular aspects, the kit comprises the NK cell therapy of the disclosure and also another cancer therapy. In some cases, the kit, in addition to the cell therapy embodiments, also includes a second cancer therapy, such as chemotherapy, hormone therapy, and/or immunotherapy, for example. The kit(s) may be tailored to a particular cancer for an individual and comprise respective second cancer therapies for the individual.

The kits may comprise suitably aliquoted compositions of the present disclosure. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also may generally contain a second, third or other additional container into which the additional components may be separately placed. However, various combinations of components may be comprised in a vial. The kits of the present invention also will typically include a means for containing the composition and any other reagent containers in close confinement for commercial sale. Such containers may include injection or blow-molded plastic containers into which the desired vials are retained.

### XI. Examples

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1

### HLA-G-Targeting Chimeric Antigen Receptors

Expression constructs were generated for production of HLA-G-targeting chimeric antigen receptor (CAR) in addition to production of the cytokine, IL-15. The expression constructs were:

| Construct | Sequence |
|---|---|
| CD8-KIR2DL4-TMD-3Z | SEQ ID NO: 1 |
| CD8-KIR2DL4-CD28-3Z | SEQ ID NO: 3 |

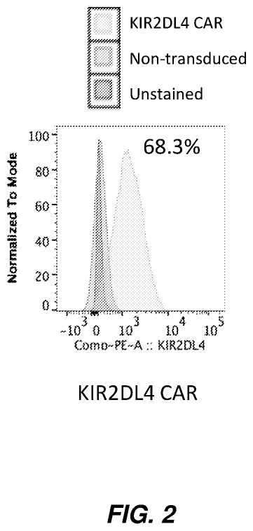

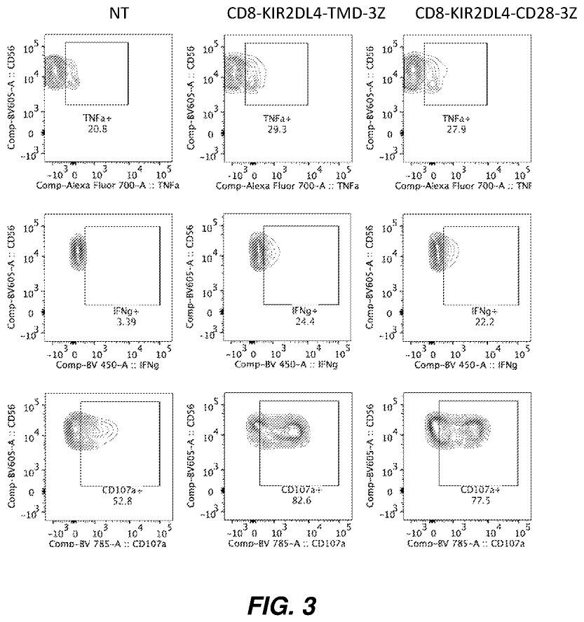

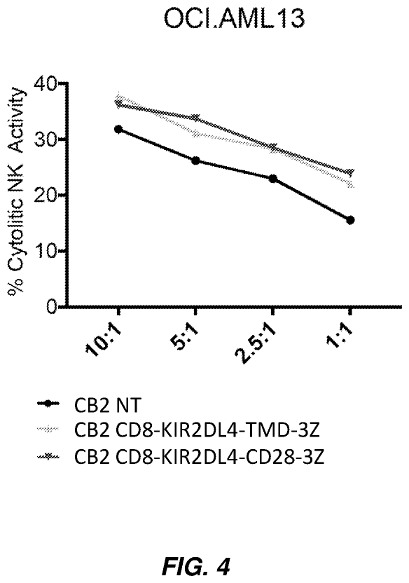

Cord blood-derived natural killer (CB-NK) cells were transduced with the different constructs, and the CAR expression was confirmed (FIG. 2). Activity of the CARs was tested against the HLA-G-positive AML cell line OCI-AML13. Activity was confirmed by intracellular cytokine staining of TNFα, IFNγ, and CD107a (FIG. 3) and by ⁵¹Cr release assay (FIG. 4).

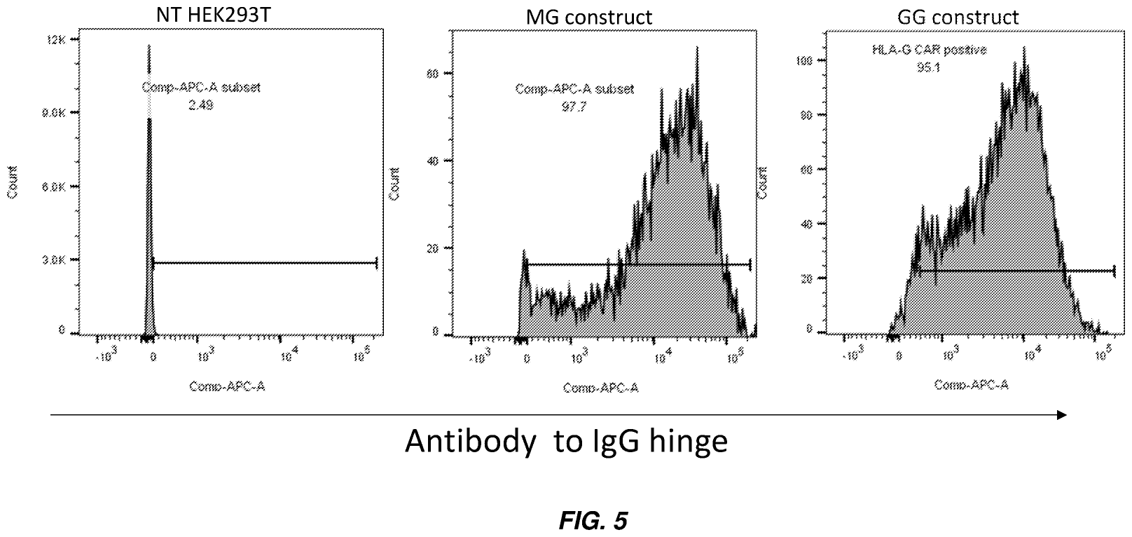

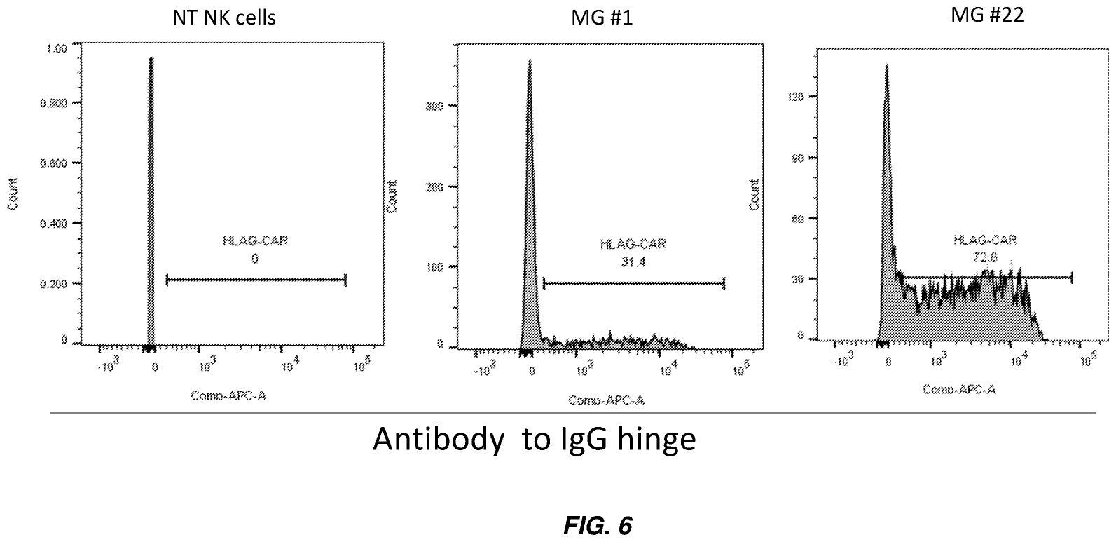

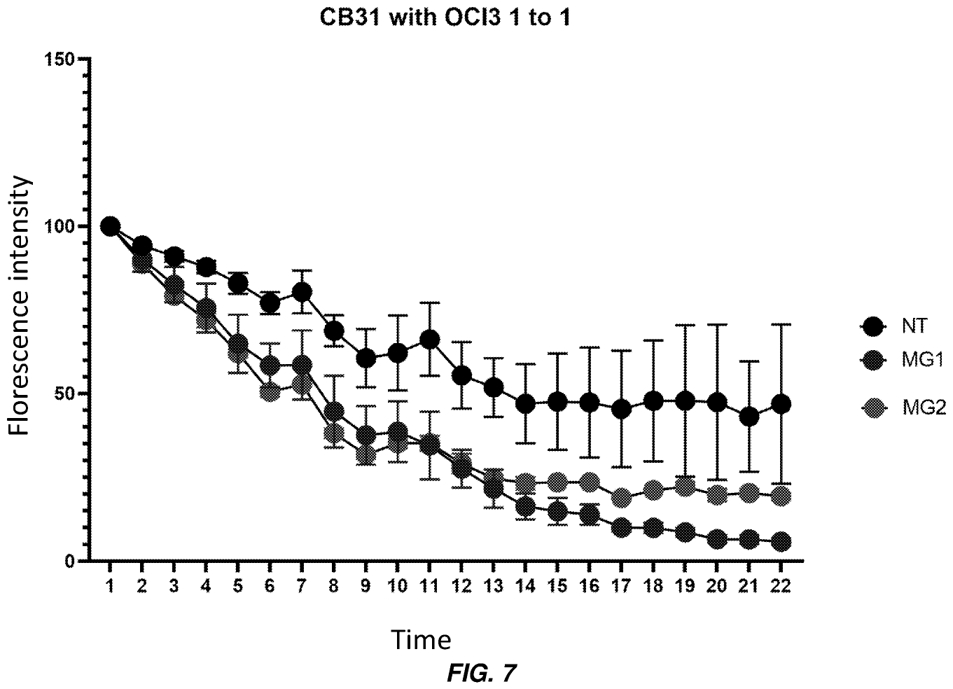

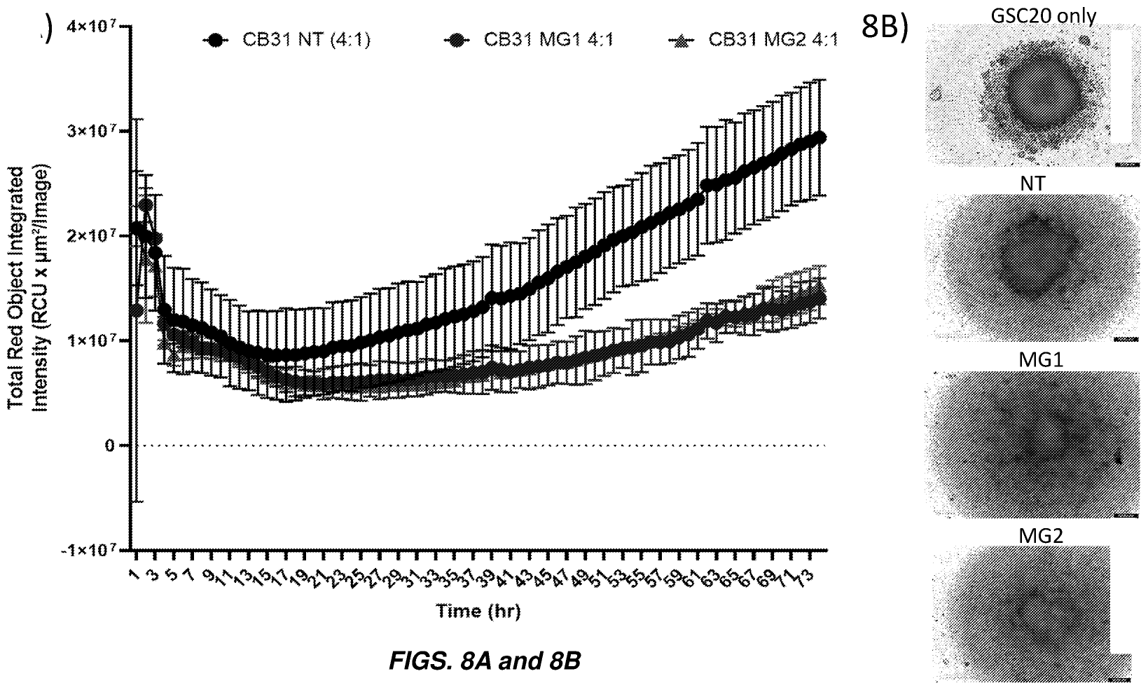

In FIG. 5, HEK293T cells were transfected with two HLA-G CAR constructs, MG construct and GG construct, derived from MEM-G/11 clone and 87G clone monoclonal antibodies against Human HLA-G, respectively. Both constructs showed positive expression on HEK293T membrane when stained with Alexa-Fluor647 affinity-purified F(ab′)2 fragment goat anti-human IgG (H+L) antibody. NK cells from cord blood were transduced with HLA-G CAR constructs (MG) (two experiments-MG1 and MG22) or were non-transduced (NT). Viral transduction efficiency was checked via staining of IgG hinge part with Alexa-Fluor647 affinity-purified F(ab′)2fragment goat anti-human IgG (H+L) antibody. Both showed positive staining compared to NT (FIG. 6). In FIG. 7, HLA-G CAR NK cells and non-transduced (NT) NK cells were cocultured at 1:1 (E:T) ratio with OCI-AML3 cells for 22 h and imaged in an Incucyte system. FIG. 7 provides a graph representing reduction in mean fluoresce intensity of the tumor target over the time. The top line is the non-transduced cells; the MG1 line is the bottom line at the end of the time period. HLA-G CAR NK cells more efficiently kill compared to NT-NK cells. In FIGS. 8A and 8B, HLA-G CAR (MG clone) expressing NK cells and non-transduced (NT) NK cells were cocultured at 4:1 (E:T) ratio with GSC20 spheroid for 73 h and imaged in an Incucyte system. FIG. 8A provides a graph representing reduction in mean fluorescense intensity of GSC20 spheroids expressing mCherry over the time cocultured with CB-NK cells transduced with HLA-G CAR (MG1 and MG2-two repeats) compared to NT-NK cells. The CB31 NT (4:1) line is the top line. FIG. 8B provides representative images of GSC20 spheroids cocultured with NT-NK, and HLA-G CAR constructs. HLA-G CAR construct (MG)-expressing NK cells and non-transduced (NT) NK cells were cocultured at 1:1 and 2:1 (E:T) ratio with OCI-AML3 cells or GSC-20 cells for 4 h and stained for CD56, CD3 and TNF-α. Cells with the MG construct show significant secretion of TNF-α compared to NT.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A polynucleotide that encodes an anti-HLA-G chimeric antigen receptor (CAR), the CAR comprising (a) a KIR2DL4 extracellular domain or an anti-HLA-G antigen binding region of an HLA-G specific antibody, (b) a transmembrane domain, and (c) an intracellular domain that is not a KIR2DL4 intracellular domain.

2. The polynucleotide of claim 1, wherein the CAR comprises the KIR2DL4 extracellular domain.

3. The polynucleotide of claim 2, wherein the KIR2DL4 extracellular domain comprises SEQ ID NO:9.

4. The polynucleotide of claim 2 or 3, wherein the KIR2DL4 extracellular domain is encoded by SEQ ID NO:7.

5. The polynucleotide of claim 2, wherein the KIR2DL4 extracellular domain comprises SEQ ID NO:10.

6. The polynucleotide of claim 2, wherein the KIR2DL4 extracellular domain is a codon optimized KIR2DL4 extracellular domain.

7. The polynucleotide of claim 6, wherein the KIR2DL4 extracellular domain is encoded by SEQ ID NO:8.

8. The polynucleotide of claim 1, wherein the CAR comprises the anti-HLA-G antigen binding region of an HLA-G specific antibody.

9. The polynucleotide of claim 1 or 8, wherein the anti-HLA-G antigen binding region of an HLA-G specific antibody comprises an scFv from an antibody clone selected from the group consisting of G233, 26-2H11, MEM-G/1, MEM-G/9, MEM-G/11, MEM-G/13, 1B8, 5E6H7, 1-2C3, 16G1, 5A6G7, 87G, and 3C/G4.

10. The polynucleotide of claim 1, 8, or 9, wherein the anti-HLA-G antigen binding region of an HLA-G specific antibody comprises an scFv from MEM-G/11 or 87G.

11. The polynucleotide of any one of claims 1-10, wherein the CAR is encoded by sequence comprising one or more of SEQ ID NO:55, 61, 67, and 73.

12. The polynucleotide of any one of claims 1-10, wherein the CAR comprises sequence comprising one or more of SEQ ID NO:50-54, 56-60, 62-66, and 68-72.

13. The polynucleotide of claim 1, further defined as an expression construct that comprises the sequence of one or more of SEQ ID NOS: 74, 76, 78, or 80.

14. The polynucleotide of claim 1, further defined as an expression construct that encodes the sequence of one or more of SEQ ID NOS: 75, 77, 79, or 81.

15. The polynucleotide of any one of claims 1-14, wherein the transmembrane domain is a transmembrane domain from CD28, the alpha chain of the T-cell receptor, beta chain of the T-cell receptor, zeta chain of the T-cell receptor, CD3 zeta, CD3 epsilon, CD3 gamma, CD3 delta, CD45, CD4, CD5, CD8, CD9, CD16, CD22, CD33, CD37, CD64, CD80, CD86, CD 134, CD137, CD154, ICOS/CD278, GITR/CD357, NKG2D, DAP10, DAP12 or any inhibitory or activating KIR.

16. The polynucleotide of claim 15, wherein the transmembrane domain is a CD28 transmembrane domain.

17. The polynucleotide of claim 16, wherein the transmembrane domain comprises SEQ ID NO:12.

18. The polynucleotide of claim 15 or 16, wherein the transmembrane domain is encoded by SEQ ID NO:11.

19. The polynucleotide of claim 15, wherein the transmembrane domain is a CD8 transmembrane domain.

20. The polynucleotide of claim 15, wherein the transmembrane domain comprises SEQ ID NO:14.

21. The polynucleotide of claim 15, wherein the transmembrane domain is encoded by SEQ ID NO:13.

22. The polynucleotide of any one of claims 1-21, wherein the intracellular domain is an intracellular domain from CD3 zeta, CD27, CD28, 4-1BB, DAP12, NKG2D, OX-40 (CD134), DAP10, CD40L, 2B4, DNAM, CS1, CD48, NKp30, NKp44, NKp46, or NKp80.

23. The polynucleotide of claim 22, wherein the intracellular domain is a CD3 zeta intracellular domain.

24. The polynucleotide of claim 23, wherein the intracellular domain comprises SEQ ID NO:16.

25. The polynucleotide of claim 23 or 24, wherein the intracellular domain is encoded by SEQ ID NO:15.

26. The polynucleotide of claim 23, wherein the intracellular domain comprises SEQ ID NO:18.

27. The polynucleotide of claim 26, wherein the intracellular domain is encoded by SEQ ID NO:17.

28. The polynucleotide of claim 22, wherein the intracellular domain is a CD28 intracellular domain.

29. The polynucleotide of claim 28, wherein the intracellular domain comprises SEQ ID NO:24.

30. The polynucleotide of claim 28 or 29, wherein the intracellular domain is encoded by SEQ ID NO:23.

31. The polynucleotide of any one of claims 1-30, wherein the CAR comprises two or more intracellular domains.

32. The polynucleotide of claim 31, wherein the two or more intracellular domains comprise a CD3 zeta intracellular domain and an additional intracellular domain selected from a CD28, DAP10, DAP12, 4-1BB, NKG2D, and 2B4 intracellular domain.

33. The polynucleotide of claim 31 or 32, wherein the two or more intracellular domains comprise a CD3 zeta intracellular domain and a CD28 intracellular domain.

34. The polynucleotide of any one of claims 1-33, further comprising a signal peptide.

35. The polynucleotide of claim 34, wherein the signal peptide is a signal peptide from CD8, CD27, granulocyte-macrophage colony-stimulating factor receptor (GMSCF-R), Ig heavy chain, a killer cell immunoglobulin-like receptor (KIR) CD3, or CD4.

36. The polynucleotide of claim 35, wherein the signal peptide is a CD8 signal peptide.

37. The polynucleotide of claim 36, wherein the signal peptide comprises SEQ ID NO:6.

38. The polynucleotide of claim 36 or 37, wherein the signal peptide is encoded by SEQ ID NO:5.

39. The polynucleotide of any one of claims 1-38, wherein the CAR comprises:
(a) a CD8 signal peptide, a KIR2DL4 extracellular domain, a CD28 transmembrane domain, and a CD3 zeta intracellular domain; or
(b) a CD8 signal peptide, a codon optimized KIR2DL4 extracellular domain, a CD8 transmembrane domain, and a CD3 zeta intracellular domain.

40. The polynucleotide of any one of claims 1-39, wherein the polynucleotide further encodes an additional polypeptide of interest.

41. The polynucleotide of claim 40, wherein the sequence encoding the additional polypeptide of interest and the sequence encoding the CAR are separated on the polynucleotide by a 2A element.

42. The polynucleotide of claim 41, wherein the 2A element is an E2A element.

43. The polynucleotide of claim 42, wherein the E2A element comprises SEQ ID NO:20.

44. The polynucleotide of claim 42 or 43, wherein the E2A element is encoded by SEQ ID NO: 19.

45. The polynucleotide of any one of claims 40-44, wherein the additional polypeptide of interest is a therapeutic protein or a protein that enhances cell activity, expansion, and/or persistence.

46. The polynucleotide of any one of claims 40-45, wherein the additional polypeptide of interest is a suicide gene, a cytokine, or a human or viral protein that enhances proliferation, expansion and/or metabolic fitness.

47. The polynucleotide of any one of claims 40-46, wherein the additional polypeptide of interest is a cytokine.

48. The polynucleotide of claim 47, wherein the cytokine is IL-15, IL-2, IL-12, IL-18, IL-21, IL-23, or IL-7.

49. The polynucleotide of claim 48, wherein the cytokine is IL-15.

50. The polynucleotide of claim 49, wherein the cytokine comprises SEQ ID NO:22.

51. The polynucleotide of claim 49 or 50, wherein the cytokine is encoded by SEQ ID NO:21.

52. The polynucleotide of any one of claims 1-51, wherein the polynucleotide encodes for a sequence having at least 95% sequence identity with SEQ ID NO:2.

53. The polynucleotide of claim 52, wherein the polynucleotide encodes for the polypeptide of SEQ ID NO:2.

54. The polynucleotide of claim 52 or 53, wherein the polynucleotide comprises a sequence having at least 95% sequence identity with SEQ ID NO:1.

55. The polynucleotide of claim 54, wherein the polynucleotide comprises SEQ ID NO:1.

56. The polynucleotide of any one of claims 1-55, wherein the polynucleotide encodes for a sequence having at least 95% sequence identity with SEQ ID NO:4.

57. The polynucleotide of claim 56, wherein the polynucleotide encodes for SEQ ID NO:4.

58. The polynucleotide of claim 56 or 57, wherein the polynucleotide comprises a sequence having at least 95% sequence identity with SEQ ID NO:3.

59. The polynucleotide of claim 58, wherein the polynucleotide comprises SEQ ID NO:3.

60. A vector comprising the polynucleotide of any one of claims 1-59.

61. The vector of claim 60, wherein the vector is a viral vector.

62. The vector of claim 61, wherein the viral vector is an adenoviral vector, adeno-associated viral vector, lentiviral vector, or retroviral vector.

63. The vector of claim 60, wherein the vector is a non-viral vector.

64. The vector of claim 63, wherein the non-viral vector is a plasmid.

65. An immune cell comprising the polynucleotide of any one of claims 1-52 or the vector of any one of claims 60-64.

66. The immune cell of claim 65, wherein the immune cell is a natural killer (NK) cell, T cell, gamma delta T cell, alpha beta T cell, invariant NKT (iNKT) cell, B cell, macrophage, mesenchymal stromal cell, or dendritic cell.

67. The immune cell of claim 66, wherein the immune cell is a NK cell

68. The immune cell of claim 67, wherein the NK cell is derived from cord blood, peripheral blood, induced pluripotent stem cells, hematopoietic stem cells, bone marrow, or from a cell line.

69. The immune cell of claim 68, wherein the NK cell is derived from a cell line, wherein the NK cell line is NK-92.

70. The immune cell of claim 68, wherein the NK cell is derived from a cord blood mononuclear cell.

71. The immune cell of any one of claims 67-70, wherein the NK cell is a CD56+NK cell.

72. The immune cell of any one of claims 67-71, wherein the NK cell expresses a recombinant cytokine.

73. The immune cell of claim 72, wherein the cytokine is IL-15, IL-2, IL-12, IL-18, IL-21, IL-7, or IL-23.

74. The immune cell of claim 73, wherein the cytokine is IL-15.

75. The immune cell of any one of claims 65-74, wherein expression of one or more endogenous genes in the immune cell has been modified.

76. The immune cell of claim 75, wherein the one or more genes comprise NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, CD5, CD7, CTLA-4, TDAG8, CD3δ, or a combination thereof.

77. A population of immune cells comprising the immune cell of any one of claims 65-76.

78. A method of killing HLA-G-positive cells in an individual, comprising administering to the individual an effective amount of cells harboring the polynucleotide of any one of claims 1-59.

79. The method of claim 78, wherein the cells harboring the polynucleotide are immune cells.

80. The method of claim 79, wherein the immune cells are NK cells, T cells, gamma delta T cells, alpha beta T cells, invariant NKT (INKT) cells, B cells, macrophages, dendritic cells, or a mixture thereof.

81. The method of claim 80, wherein the immune cells comprise NK cells, wherein the NK cells are derived from cord blood, peripheral blood, induced pluripotent stem cells, hematopoietic stem cells, bone marrow, from a cell line, or a mixture thereof.

82. The method of claim 81, wherein the NK cells are derived from cord blood mononuclear cells.

83. The method of any one of claims 79-82, wherein the immune cells are allogeneic with respect to the individual.

84. The method of any one of claims 79-82, wherein the immune cells are autologous with respect to the individual.

85. The method of any one of claims 78-84, wherein the individual is human.

86. The method of any one of claims 78-85, wherein the individual has cancer.

87. The method of claim 86, wherein the individual has breast cancer, acute myeloid leukemia, multiple myeloma, or glioblastoma.

88. The method of any one of claims 78-87, wherein the cells harboring the polynucleotide are administered to the individual once or more than once.

89. The method of claim 88, wherein the duration of time between administrations of the cells harboring the polynucleotide to the individual is 1-24 hours, 1-7 days, 1-4 weeks, 1-12 months, or one or more years.

90. The method of any one of claims 78-89, further comprising the step of providing to the individual an effective amount of an additional therapy.

91. The method of claim 90, wherein the additional therapy comprises surgery, radiation, gene therapy, immunotherapy, or hormone therapy.

92. The method of any one of claims 78-91, wherein the cells harboring the polynucleotide are administered to the individual by injection, intravenously, intraarterially, intraperitoneally, intratracheally, intratumorally, intramuscularly, endoscopically, intralesionally, intracranially, percutaneously, subcutaneously, regionally, by perfusion, in a tumor microenvironment, or a combination thereof.

93. The method of any one of claims 78-92, further comprising identifying HLA-G-positive cancer in the individual.

94. The method of any one of claims 78-93, further comprising producing the cells harboring the polynucleotide.

95. As a composition of matter, a polynucleotide comprising SEQ ID NO:1, SEQ ID NO:3, SEQ ID NO:74, SEQ ID NO:76, SEQ ID NO:78, or SEQ ID NO:80.

96. As a composition of matter, a polypeptide comprising SEQ ID NO:2, SEQ ID NO:4, SEQ ID NO:75, SEQ ID NO:77, SEQ ID NO: 79, or SEQ ID NO:81.

97. An antibody-drug conjugate, wherein the antibody portion is encoded by SEQ ID NO:55, SEQ ID NO:61, SEQ ID NO:67, or SEQ ID NO:73.

98. An antibody-drug conjugate, wherein the antibody portion comprises sequence comprising one or more of SEQ ID NOs:50-54, 56-60, 62-66, and 68-72.

99. The antibody-drug conjugate of claim 97 or 98, wherein the antibody is conjugated to a toxin, chemotherapeutic, radionuclide, small molecule, and/or ricin A chain.

100. The antibody-drug conjugate of claim 99, wherein the toxin is a cholera toxin and/or pertussis toxin.

101. A bi-specific or multi-specific antibody, comprising an HLA-G-specific antibody.

102. The antibody of claim 101, wherein an anti-HLA-G antigen binding region of the HLA-G specific antibody comprises an scFv from an antibody clone selected from the group consisting of G233, 26-2H11, MEM-G/1, MEM-G/9, MEM-G/11, MEM-G/13, 1B8, 5E6H7, 1-2C3, 16G1, 5A6G7, 87G, and 3C/G4.

103. The antibody of claim 101 or 102, wherein part or all of the HLA-G-specific antibody is encoded by SEQ ID NO:55, 61, 67, or 73, or a sequence that is at least 70, 75, 80, 85, 90, 95, 96, 97, 98, 99, or more % identical to SEQ ID NO: 55, 61, 67, or 73.

104. The antibody of any one of claims 101-103, wherein the HLA-G-specific antibody comprises SEQ ID NO:50-54, 56-60, 62-66, or 68-72, or a sequence that is at least 70, 75, 80, 85, 90, 95, 96, 97, 98, 99, or more % identical to SEQ ID NO: 50-54, 56-60, 62-66, or 68-72.

105. The antibody of any one of claims 101-104, wherein the antibody comprises a CD3-specific antibody.

106. A cell, comprising the antibody-drug conjugate of any one of claims 97-100 and/or comprising the antibody of any one of claims 101-105.

107. A plurality of cells of claim 106, wherein the cells are immune effector cells.

108. The plurality of claim 107, wherein the immune effector cells are NK cells, NK T cells, invariant NKT cells, gamma delta T cells, alpha beta T cells, regulatory T cells, B cells, macrophages, mesenchymal stromal cells (MSCs), dendritic cells, or a mixture thereof.

109. The plurality of claim 107 or 108, wherein the plurality is comprised in a pharmaceutically acceptable excipient.

110. A method of treating or preventing cancer in an individual, comprising the step of delivering to the individual an effective amount of the cells of any one of claims 107-109.
